SEQUENCE LISTING (163 sequences)

SEQ ID NO: 1 - DNA, 1131 bp, Saccharomyces cerevisiae
  source 1..1131: mol_type = other DNA
atgtcgaagggaaaggttttgctggttctttacgaaggtggtaagcatgctgaagagcaggaaaagttattggggtgtattgaaaatgaacttggtatcagaaatttcattgaagaacagggatacgagttggttactaccattgacaaggaccctgagccaacctcaacggtagacagggagttgaaagacgctgaaattgtcattactacgccctttttccccgcctacatctcgagaaacaggattgcagaagctcctaacctgaagctctgtgtaaccgctggcgtcggttcagaccatgtcgatttagaagctgcaaatgaacggaaaatcacggtcaccgaagttactggttctaacgtcgtttctgtcgcagagcacgttatggccacaattttggttttgataagaaactataatggtggtcatcaacaagcaattaatggtgagtgggatattgccggcgtggctaaaaatgagtatgatctggaagacaaaataatttcaacggtaggtgccggtagaattggatatagggttctggaaagattggtcgcatttaatccgaagaagttactgtactacgactaccaggaactacctgcggaagcaatcaatagattgaacgaggccagcaagcttttcaatggcagaggtgatattgttcagagagtagagaaattggaggatatggttgctcagtcagatgttgttaccatcaactgtccattgcacaaggactcaaggggtttattcaataaaaagcttatttcccacatgaaagatggtgcatacttggtgaataccgctagaggtgctatttgtgtcgcagaagatgttgccgaggcagtcaagtctggtaaattggctggctatggtggtgatgtctgggataagcaaccagcaccaaaagaccatccctggaggactatggacaataaggaccacgtgggaaacgcaatgactgttcatatcagtggcacatctctggatgctcaaaagaggtacgctcagggagtaaagaacatcctaaatagttacttttccaaaaagtttgattaccgtccacaggatattattgtgcagaatggttcttatgccaccagagcttatggacagaagaaataa

SEQ ID NO: 2 - protein, 376 aa, Saccharomyces cerevisiae
MSKGKVLLVLYEGGKHAEEQEKLLGCIENELGIRNFIEEQGYELVTTIDKDPEPTSTVDRELKDAEIVITTPFFPAYISRNRIAEAPNLKLCVTAGVGSDHVDLEAANERKITVTEVTGSNVVSVAEHVMATILVLIRNYNGGHQQAINGEWDIAGVAKNEYDLEDKIISTVGAGRIGYRVLERLVAFNPKKLLYYDYQELPAEAINRLNEASKLFNGRGDIVQRVEKLEDMVAQSDVVTINCPLHKDSRGLFNKKLISHMKDGAYLVNTARGAICVAEDVAEAVKSGKLAGYGGDVWDKQPAPKDHPWRTMDNKDHVGNAMTVHISGTSLDAQKRYAQGVKNILNSYFSKKFDYRPQDIIVQNGSYATRAYGQKK

SEQ ID NO: 3 - DNA, 1129 bp, Saccharomyces cerevisiae
  source 1..1129: mol_type = other DNA
atgtcgaagggaaaggttttgctggttctttatgaaggtggtaagcatgctgaagagcaggaaaagttattggggtgtattgaaaatgaacttggtatcagaaatttcattgaagaacagggatacgagttggttactaccattgacaaggaccctgagccaacctcaacggtagacagggagttgaaagacgctgaaattgtcattactacgccctttttccccgcctacatctcgagaaacaggattgcagaagctcctaacctgaagctctgtgtaaccgctggcgtcggttcagaccatgtcgatttagaagctgcaaatgaacggaaaatcacggtcaccgaagttactggttctaacgtcgtttctgtcgcagagcacgttatggccacaattttggttttgataagaaactataatggtggtcatcaataagcaattaatggtgagtgggatattgccggcgtggctaaaaaatgagtatgatctggaagacaaaataatttcaacggtaggtgccggtagaattggatatagggttctggaaagattggtcgcatttaatccgaagaagttactgtactacgactaccaggaactacctgcggaagcaatcaatagattgaacgaggccagcaagcttttcaatggcagaggtgatattgttcagagagtagagaaattggaggatatggttgctcagtcagatgttgttaccatcaactgtccattgcacaaggactcaaggggtttattcaataaaaagcttatttcccacatgaaagatggtgcatacttggtgaataccgctagaggtgctatttgtgtcgcagaagatgttgccgaggcagtcaagtctggtaaattggctggctatggtggtgatgtctgggataagcaaccagcaccaaaagaccatccctggaggactatggacaataaggaccacgtgggaaacgcaatgactgttcatatcagtggcacatctctgcatgctcaaaagaggtacgctcagggagtaaagaacatcctaaatagttacttttccaaaaagtttgattaccgtccacaggatattattgtgcagaatggttcttatgccaccagagcttatggacagaagaaa

SEQ ID NO: 4 - DNA, 1176 bp, Saccharomyces cerevisiae
  source 1..1176: mol_type = other DNA
atgtctgctgctgctgatagattaaacttaacttccggccacttgaatgctggtagaaagagaagttcctcttctgtttctttgaaggctgccgaaaagcctttcaaggttactgtgattggatctggtaactggggtactactattgccaaggtggttgccgaaaattgtaagggatacccagaagttttcgctccaatagtacaaatgtgggtgttcgaagaagagatcaatggtgaaaaattgactgaaatcataaatactagacatcaaaacgtgaaatacttgcctggcatcactctacccgacaatttggttgctaatccagacttgattgattcagtcaaggatgtcgacatcatcgttttcaacattccacatcaatttttgccccgtatctgtagccaattgaaaggtcatgttgattcacacgtcagagctatctcctgtctaaagggttttgaagttggtgctaaaggtgtccaattgctatcctcttacatcactgaggaactaggtattcaatgtggtgctctatctggtgctaacattgccaccgaagtcgctcaagaacactggtctgaaacaacagttgcttaccacattccaaaggatttcagaggcgagggcaaggacgtcgaccataaggttctaaaggccttgttccacagaccttacttccacgttagtgtcatcgaagatgttgctggtatctccatctgtggtgctttgaagaacgttgttgccttaggttgtggtttcgtcgaaggtctaggctggggtaacaacgcttctgctgccatccaaagagtcggtttgggtgagatcatcagattcggtcaaatgtttttcccagaatctagagaagaaacatactaccaagagtctgctggtgttgctgatttgatcaccacctgcgctggtggtagaaacgtcaaggttgctaggctaatggctacttctggtaaggacgcctgggaatgtgaaaaggagttgttgaatggccaatccgctcaaggtttaattacctgcaaagaagttcacgaatggttggaaacatgtggctctgtcgaagacttcccattatttgaagccgtataccaaatcgtttacaacaactacccaatgaagaacctgccggacatgattgaagaattagatctacatgaagattag

SEQ ID NO: 5 - protein, 391 aa, Saccharomyces cerevisiae
MSAAADRLNLTSGHLNAGRKRSSSSVSLKAAEKPFKVTVIGSGNWGTTIAKVVAENCKGYPEVFAPIVQMWVFEEEINGEKLTEIINTRHQNVKYLPGITLPDNLVANPDLIDSVKDVDIIVFNIPHQFLPRICSQLKGHVDSHVRAISCLKGFEVGAKGVQLLSSYITEELGIQCGALSGANIATEVAQEHWSETTVAYHIPKDFRGEGKDVDHKVLKALFHRPYFHVSVIEDVAGISICGALKNVVALGCGFVEGLGWGNNASAAIQRVGLGEIIRFGQMFFPESREETYYQESAGVADLITTCAGGRNVKVARLMATSGKDAWECEKELLNGQSAQGLITCKEVHEWLETCGSVEDFPLFEAVYQIVYNNYPMKNLPDMIEELDLHED

SEQ ID NO: 6 - DNA, 1320 bp, Saccharomyces cerevisiae
  source 1..1320: mol_type = other DNA
atgcttgctgtcagaagattaacaagatacacattccttaagcgaacgcatccggtgttatatactcgtcgtgcatataaaattttgccttcaagatctactttcctaagaagatcattattacaaacacaactgcactcaaagatgactgctcatactaatatcaaacagcacaaacactgtcatgaggaccatcctatcagaagatcggactctgccgtgtcaattgtacatttgaaacgtgcgcccttcaaggttacagtgattggttctggtaactgggggaccaccatcgccaaagtcattgcggaaaacacagaattgcattcccatatcttcgagccagaggtgagaatgtgggtttttgatgaaaagatcggcgacgaaaatctgacggatatcataaatacaagacaccagaacgttaaatatctacccaatattgacctgccccataatctagtggccgatcctgatcttttacactccatcaagggtgctgacatccttgttttcaacatccctcatcaatttttaccaaacatagtcaaacaattgcaaggccacgtggcccctcatgtaagggccatctcgtgtctaaaagggttcgagttgggctccaagggtgtgcaattgctatcctcctatgttactgatgagttaggaatccaatgtggcgcactatctggtgcaaacttggcaccggaagtggccaaggagcattggtccgaaaccaccgtggcttaccaactaccaaaggattatcaaggtgatggcaaggatgtagatcataagattttgaaattgctgttccacagaccttacttccacgtcaatgtcatcgatgatgttgctggtatatccattgccggtgccttgaagaacgtcgtggcacttgcatgtggtttcgtagaaggtatgggatggggtaacaatgcctccgcagccattcaaaggctgggtttaggtgaaattatcaagttcggtagaatgtttttcccagaatccaaagtcgagacctactatcaagaatccgctggtgttgcagatctgatcaccacctgctcaggcggtagaaacgtcaaggttgccacatacatggccaagaccggtaagtcagccttggaagcagaaaaggaattgcttaacggtcaatccgcccaagggataatcacatgcagagaagttcacgagtggctacaaacatgtgagttgacccaagaattcccattattcgaggcagtctaccagatagtctacaacaacgtccgcatggaagacctaccggagatgattgaagagctagacatcgatgacgaa

SEQ ID NO: 7 - protein, 440 aa, Saccharomyces cerevisiae
MLAVRRLTRYTFLKRTHPVLYTRRAYKILPSRSTFLRRSLLQTQLHSKMTAHTNIKQHKHCHEDHPIRRSDSAVSIVHLKRAPFKVTVIGSGNWGTTIAKVIAENTELHSHIFEPEVRMWVFDEKIGDENLTDIINTRHQNVKYLPNIDLPHNLVADPDLLHSIKGADILVFNIPHQFLPNIVKQLQGHVAPHVRAISCLKGFELGSKGVQLLSSYVTDELGIQCGALSGANLAPEVAKEHWSETTVAYQLPKDYQGDGKDVDHKILKLLFHRPYFHVNVIDDVAGISIAGALKNVVALACGFVEGMGWGNNASAAIQRLGLGEIIKFGRMFFPESKVETYYQESAGVADLITTCSGGRNVKVATYMAKTGKSALEAEKELLNGQSAQGIITCREVHEWLQTCELTQEFPLFEAVYQIVYNNVRMEDLPEMIEELDIDDE

SEQ ID NO: 8 - DNA, 2376 bp, Bifidobacterium adolescentis
  source 1..2376: mol_type = other DNA
atggcagcagttgatgcaacggcggtctcccaggaggaacttgaggctaaggcttgggaaggcttcaccgagggcaactggcagaaggacattgatgtccgcgacttcatccagaagaactacacgccatatgagggcgacgagtccttcctggctgacgccaccgacaagaccaagcacctgtggaagtatctggacgacaactatctgtccgtggagcgcaagcagcgcgtctacgacgtggacacccacaccccggcgggcatcgacgccttcccggccggctacatcgattccccggaagtcgacaatgtgattgtcggtctgcagaccgatgtgccgtgcaagcgcgccatgatgccgaacggcggctggcgtatggtcgagcaggccatcaaggaagccggcaaggagcccgatccggagatcaagaagatcttcaccaagtaccgcaagacccacaacgacggcgtcttcggcgtctacaccaagcagatcaaggtagctcgccacaacaagatcctcaccggcctgccggatgcctacggccgtggccgcatcatcggcgattaccgtcgtgtggccctgtacggcgtgaacgcgctgatcaagttcaagcagcgcgacaaggactccatcccgtaccgcaacgacttcaccgagccggagatcgagcactggatccgcttccgtgaggagcatgacgagcagatcaaggccctgaagcagctgatcaacctcggcaacgagtacggcctcgacctgtcccgcccggcacagaccgcacaggaagccgtgcagtggacctacatgggctacctcgcctccgtcaagagccaggacggcgccgccatgtccttcggccgtgtctccaccttcttcgacgtctacttcgagcgcgacctgaaggccggcaagatcaccgagaccgacgcacaggagatcatcgataacctggtcatgaagctgcgcatcgtgcgcttcctgcgcaccaaggattacgacgcgatcttctccggcgatccgtactgggcgacttggtccgacgccggcttcggcgacgacggccgtaccatggtcaccaagacctcgttccgtctgctcaacaccctgaccctcgagcacctcggacctggcccggagccgaacatcaccatcttctgggatccgaagctgccggaagcctacaagcgcttctgcgcccgaatctccatcgacacctcggccatccagtacgagtccgataaggaaatccgctcccactggggcgacgacgccgccatcgcatgctgcgtctccccgatgcgcgtgggcaagcagatgcagttcttcgccgcccgtgtgaactccgccaaggccctgctgtacgccatcaacggcggacgcgacgagatgaccggcatgcaggtcatcgacaagggcgtcatcgacccgatcaagccggaagccgatggcacgctggattacgagaaggtcaaggccaactacgagaaggccctcgaatggctgtccgagacctatgtgatggctctgaacatcatccattacatgcatgataagtacgcttacgagtccatcgagatggctctgcacgacaaggaagtgtaccgcaccctcggctgcggcatgtccggcctgtcgatcgcggccgactccctgtccgcatgcaagtacgccaaggtctacccgatctacaacaaggacgccaagaccacgccgggccacgagaacgagtacgtcgaaggcgccgatgacgatctgatcgtcggctaccgcaccgaaggcgacttcccgctgtacggcaacgatgatgaccgtgccgacgacatcgccaagtgggtcgtctccaccgtcatgggccaggtcaagcgtctgccggtgtaccgcgacgccgtcccgacccagtccatcctgaccatcacctccaatgtggaatacggcaaggccaccggcgccttcccgtccggccacaagaagggcaccccgtacgctccgggcgccaacccggagaacggcatggactcccacggcatgctgccgtccatgttctccgtcggcaagatcgactacaacgacgctcttgacggcatctcgctgaccaacaccatcacccctgatggtctgggccgcgacgaggaagagcgtatcggcaacctcgttggcatcctggacgccggcaacggccacggcctgtaccacgccaacatcaacgtgctgcgcaaggagcagctcgaggatgccgtcgagcatccggagaagtacccgcacctgaccgtgcgcgtctccggctacgcggtgaacttcgtcaagctcaccaaggaacagcagctcgacgtgatctcccgtacgttccaccagggcgctgtcgtcgactga

SEQ ID NO: 9 - protein, 791 aa, Bifidobacterium adolescentis
MAAVDATAVSQEELEAKAWEGFTEGNWQKDIDVRDFIQKNYTPYEGDESFLADATDKTKHLWKYLDDNYLSVERKQRVYDVDTHTPAGIDAFPAGYIDSPEVDNVIVGLQTDVPCKRAMMPNGGWRMVEQAIKEAGKEPDPEIKKIFTKYRKTHNDGVFGVYTKQIKVARHNKILTGLPDAYGRGRIIGDYRRVALYGVNALIKFKQRDKDSIPYRNDFTEPEIEHWIRFREEHDEQIKALKQLINLGNEYGLDLSRPAQTAQEAVQWTYMGYLASVKSQDGAAMSFGRVSTFFDVYFERDLKAGKITETDAQEIIDNLVMKLRIVRFLRTKDYDAIFSGDPYWATWSDAGFGDDGRTMVTKTSFRLLNTLTLEHLGPGPEPNITIFWDPKLPEAYKRFCARISIDTSAIQYESDKEIRSHWGDDAAIACCVSPMRVGKQMQFFAARVNSAKALLYAINGGRDEMTGMQVIDKGVIDPIKPEADGTLDYEKVKANYEKALEWLSETYVMALNIIHYMHDKYAYESIEMALHDKEVYRTLGCGMSGLSIAADSLSACKYAKVYPIYNKDAKTTPGHENEYVEGADDDLIVGYRTEGDFPLYGNDDDRADDIAKWVVSTVMGQVKRLPVYRDAVPTQSILTITSNVEYGKATGAFPSGHKKGTPYAPGANPENGMDSHGMLPSMFSVGKIDYNDALDGISLTNTITPDGLGRDEEERIGNLVGILDAGNGHGLYHANINVLRKEQLEDAVEHPEKYPHLTVRVSGYAVNFVKLTKEQQLDVISRTFHQGAVVD

SEQ ID NO: 10 - DNA, 879 bp, Bifidobacterium adolescentis
  source 1..879: mol_type = other DNA
atgtctgaacatattttccgttccacgaccagacacatgctgagggattccaaggactacgtcaatcagacgctgatgggaggcctgtccggattcgaatcgccaatcggcttggaccgtctcgaccgcatcaaggcgttgaaaagcggcgatatcggtttcgtgcactcgtgggacatcaacacttccgtggatggtcctggcaccagaatgaccgtgttcatgagcggatgccctctgcgctgccagtactgccagaatccggatacttggaagatgcgcgacggcaagcccgtctactacgaagccatggtcaagaaaatcgagcggtatgccgatttattcaaggccaccggcggcggcatcactttctccggcggcgaatccatgatgcagccggctttcgtgtcacgcgtgttccatgccgccaagcagatgggagtgcatacctgcctcgacacgtccggattcctcggggcgagctacaccgatgacatggtggatgacatcgacctgtgcctgcttgacgtcaaatccggcgatgaggagacctaccataaggtgaccggcggcatcctgcagccgaccatcgacttcggacagcgtctggccaaggcaggcaagaagatctgggtgcgtttcgtgctcgtgccgggcctcacatcctccgaagaaaacgtcgagaacgtggcgaagatctgcgagaccttcggcgacgcgttggaacatatcgacgtattgcccttccaccagcttggccgtccgaagtggcacatgctgaacatcccatacccgttggaggaccagaaaggcccgtccgcggcaatgaaacaacgtgtggtcgagcagttccagtcgcacggcttcaccgtgtactaa

SEQ ID NO: 11 - protein, 292 aa, Bifidobacterium adolescentis
MSEHIFRSTTRHMLRDSKDYVNQTLMGGLSGFESPIGLDRLDRIKALKSGDIGFVHSWDINTSVDGPGTRMTVFMSGCPLRCQYCQNPDTWKMRDGKPVYYEAMVKKIERYADLFKATGGGITFSGGESMMQPAFVSRVFHAAKQMGVHTCLDTSGFLGASYTDDMVDDIDLCLLDVKSGDEETYHKVTGGILQPTIDFGQRLAKAGKKIWVRFVLVPGLTSSEENVENVAKICETFGDALEHIDVLPFHQLGRPKWHMLNIPYPLEDQKGPSAAMKQRVVEQFQSHGFTVY

SEQ ID NO: 12 - DNA, 2733 bp, Bifidobacterium adolescentis
  source 1..2733: mol_type = other DNA
atggcagacgcaaagaagaaggaagagccgaccaagccgactccggaagagaagctcgccgcagccgaggctgaggtcgacgctctggtcaagaagggcctgaaggctcttgatgaattcgagaagctcgatcagaagcaggttgaccacatcgtggccaaggcttccgtcgcagccctgaacaagcacttggtgctcgccaagatggccgtcgaggagacccaccgtggtctggtcgaagacaaggccaccaagaacatcttcgcctgcgagcatgtcaccaactacctggctggtcagaagaccgtcggcatcatccgcgaggacgacgtgctgggcatcgacgaaatcgccgagccggttggcgtcgtcgctggcgtgaccccggtcaccaacccgacctccaccgccatcttcaagtcgctgatcgcactgaagacccgctgcccgatcatcttcggcttccacccgggcgcacagaactgctccgtcgcggccgccaagatcgttcgcgatgccgctatcgcagcaggcgctcctgagaactgtattcagtggatcgagcatccgtccatcgaggccactggcgccctgatgaagcatgatggtgtcgccaccatcctcgccaccggtggtccgggcatggtcaaggccgcatactcctccggcaagccggccctgggcgtcggcgcgggcaatgctccggcatacgttgacaagaacgtcgacgtcgtgcgtgcagccaacgatctgattctttccaagcacttcgattacggcatgatctgcgctaccgagcaggccatcatcgccgacaaggacatctacgctccgctcgttaaggaactcaagcgtcgcaaggcctatttcgtgaacgctgacgagaaggccaagctcgagcagtacatgttcggctgcaccgcttactccggacagaccccgaagctcaactccgtggtgccgggcaagtccccgcagtacatcgccaaggccgccggcttcgagattccggaagacgccaccatccttgccgctgagtgcaaggaagtcggcgagaacgagccgctgaccatggagaagcttgctccggtccaggccgtgctgaagtccgacaacaaggaacaggccttcgagatgtgcgaagccatgctgaagcatggcgccggccacaccgccgccatccacaccaacgaccgtgacctggtccgcgagtacggccagcgcatgcacgcctgccgtatcatctggaactccccgagctccctcggcggcgtgggcgacatctacaacgccatcgctccgtccctgaccctgggctgcggctcctacggcggcaactccgtgtccggcaacgtccaggcagtcaacctcatcaacatcaagcgcatcgctcggaggaacaacaacatgcagtggttcaagattccggccaagacctacttcgagccgaacgccatcaagtacctgcgcgacatgtacggcatcgaaaaggccgtcatcgtgtgcgataaggtcatggagcagctcggcatcgttgacaagatcatcgatcagctgcgtgcacgttccaaccgcgtgaccttccgtatcatcgattatgtcgagccggagccgagcgtggagaccgtcgaacgtggcgccgccatgatgcgcgaggagttcgagccggataccatcatcgccgtcggcggtggttccccgatggatgcgtccaagattatgtggctgctgtacgagcacccggaaatctccttctccgatgtgcgtgagaagttcttcgatatccgtaagcgcgcgttcaagattccgccgctgggcaagaaggccaagctggtctgcattccgacttcttccggcaccggttccgaagtcacgccgttcgctgtgattaccgaccacaagaccggctataagtacccgatcaccgattacgcgctgaccccgtccgtcgctatcgtcgatccggtgctggcacgtactcagccgcgcaagctggcttccgatgctggtttcgatgctctgacccacgcttttgaggcttatgtgtccgtgtatgccaacgacttcaccgatggtatggcattgcacgctgccaagctggtttgggacaacctcgctgagtccgtcaatggcgagccgggtgaggagaagacccgtgcccaggagaagatgcataatgccgccaccatggccggcatggctttcggctccgccttcctcggcatgtgccacggcatggcccacaccattggtgcactgtgccacgttgcccacggtcgtaccaactccatcctcctgccgtacgtgatccgttacaacggttccgtcccggaggagccgaccagctggccgaagtacaacaagtacatcgctccggaacgctaccaggagatcgccaagaaccttggcgtgaacccgggcaagactccggaagagggcgtcgagaacctggccaaggctgttgaggattaccgtgacaacaagctcggtatgaacaagagcttccaggagtgcggtgtggatgaggactactattggtccatcatcgaccagatcggcatgcgcgcctacgaagaccagtgcgcaccggcgaacccgcgtatcccgcagatcgaggatatgaaggatatcgccattgccgcctactacggcgtcagccaggcggaaggccacaagctgcgcgtccagcgtcagggcgaagccgctacggaggaagcttccgagcgcgcctga

SEQ ID NO: 13 - protein, 910 aa, Bifidobacterium adolescentis
MADAKKKEEPTKPTPEEKLAAAEAEVDALVKKGLKALDEFEKLDQKQVDHIVAKASVAALNKHLVLAKMAVEETHRGLVEDKATKNIFACEHVTNYLAGQKTVGIIREDDVLGIDEIAEPVGVVAGVTPVTNPTSTAIFKSLIALKTRCPIIFGFHPGAQNCSVAAAKIVRDAAIAAGAPENCIQWIEHPSIEATGALMKHDGVATILATGGPGMVKAAYSSGKPALGVGAGNAPAYVDKNVDVVRAANDLILSKHFDYGMICATEQAIIADKDIYAPLVKELKRRKAYFVNADEKAKLEQYMFGCTAYSGQTPKLNSVVPGKSPQYIAKAAGFEIPEDATILAAECKEVGENEPLTMEKLAPVQAVLKSDNKEQAFEMCEAMLKHGAGHTAAIHTNDRDLVREYGQRMHACRIIWNSPSSLGGVGDIYNAIAPSLTLGCGSYGGNSVSGNVQAVNLINIKRIARRNNNMQWFKIPAKTYFEPNAIKYLRDMYGIEKAVIVCDKVMEQLGIVDKIIDQLRARSNRVTFRIIDYVEPEPSVETVERGAAMMREEFEPDTIIAVGGGSPMDASKIMWLLYEHPEISFSDVREKFFDIRKRAFKIPPLGKKAKLVCIPTSSGTGSEVTPFAVITDHKTGYKYPITDYALTPSVAIVDPVLARTQPRKLASDAGFDALTHAFEAYVSVYANDFTDGMALHAAKLVWDNLAESVNGEPGEEKTRAQEKMHNAATMAGMAFGSAFLGMCHGMAHTIGALCHVAHGRTNSILLPYVIRYNGSVPEEPTSWPKYNKYIAPERYQEIAKNLGVNPGKTPEEGVENLAKAVEDYRDNKLGMNKSFQECGVDEDYYWSIIDQIGMRAYEDQCAPANPRIPQIEDMKDIAIAAYYGVSQAEGHKLRVQRQGEAATEEASERA

SEQ ID NO: 14 - DNA, 2703 bp, Leuconostoc mesenteroides
  source 1..2703: mol_type = other DNA
atggcagaagcaattgcaaagaaacccgcaaaaagggttttgacccctgaagaaaaagcggaattacaaacacaagctgagaagatgactgaggtattgattgaaaaatcacaaaaggcattgtctgaattttcaacattttcgcaagaacaagttgataaaattgttgcagctatggccttggcaggttctgagaattcacttctgttagcccatgctgctcacgacgagactggacgtggggttgtggaagataaggatacgaaaaatcgtttcgcctcagaatcagtttataacgctattaagtttgataagactgtgggtgttattagtgaagacaagattcaaggtaaggtagaattagcagccccacttggtattttggctggaatcgtcccaacgacaaatccaacgtcgacaactattttcaaatcaatgttgacagcaaagacacgtaacacaattatctttgctttccatccccaggctcaaaaagcatcggttcttgctgcaaaaattgtttatgatgctgctgttaaagcaggcgcaccggaaaactttatccaatggattgaaaagccttcactttatgcaacaagtgcgctgatacaaaatcctcacattgcttcaattctagctactggtgggccatcaatggttaatgcagctttgaagtcaggaaatccatccatgggtgtcggtgctggaaacggtgcagtttatattgatgcaactgttgacacagatcgtgccgtgtccgatttgttgttatcaaagcgtttcgataatggcatgatttgtgccacagaaaactcagccgttattcaagcaccaatctatgacgaaattttaactaagttacaagaacaaggtgcataccttgttcctaagaaagactacaaaaaaattgctgattatgtatttaagcctaacgcagagggatttggtattgctggtcctgttgctggtatgtcaggacgttggattgctgagcaagcaggcgtaaagattcctgatggtaaagatgtacttttgttcgaattagatcagaagaacataggtgaagcgttatcttctgaaaagttatcgccattactttcaatttataaagttgagaagcgtgaagaagctattgagactgttcaatccttgttaaactatcaaggcgcggggcacaacgcagcaattcaaattggttcacaagatgatccattcattaaagagtatgctgacgctattggtgcatcacgtattttggttaaccaacctgactcaatcggtggtgttggagatatttacacagatgctatgcgtccatcgttgacacttggtaccggatcatgggggaagaattcattgtctcataacttatcaacatacgacttacttaatattaagaccgtggctcgccgccgtaatcgtcctcaatgggttcgtttacctaaggaagtttactacgaagccaatgccattacttacttacaagacttgcctactataaaccgtgcatttattgtcgctgatcctggtatggttcagttcggatttgttggcagagtactaggtcaacttgagttacgtcaagaacaggttgaaacaaatatctatggttcagttaagcctgacccaactttgtcacaagctgttgaaattgctcgccaaatggcagacttcaaaccagatacagttattttacttggcggtggttcggcacttgacgctggtaaaattggtcggttcttgtacgaatactcgacacgccatgaaggaattttagaagatgacgaggcgattaaagatctattcttagaactacaacaaaagtttatggatattcgtaagcgaatcgttaagttttaccacgcacgtttgacacaaatggttgcgattccaacaacttcaggtactggatcagaagtcacaccatttgccgttattacagatgatgaaacacatgtaaagtatccactagccgattatgaattgacaccggaagttgctattgttgatccagaatttgttatgaccgtaccacaacacacggtatcttggtcaggattagatgctttgtcacatgctttggaatcgtatgtctcagtgatggcttctgaattcacacgtccttgggcattacaagctattaagttgatttttgataacttaacaaattcatacaattatgatcctaaacacccaactaaggaaggtcagaatgcacgcacaaagatgcactatgcgtcaacattggctggtatgtcatttgcgaatgccttcttgggacttaaccactcactagcacacaaaactggtggagaattcggactacctcacggtatggcaatcgctattgcaatgccacatgtgattaagtttaatgcggtaacaggaaatgtaaagcgcacaccatacccacgttacgaaacctatacagcacaaaaagattatgctgatattgcacgttacttaggtttgaaaggtgaaacagatgctgaattggtcgatgtattgattgcagaaatcaagaagttggctgcatcagtgggtgtcaatcaaacactatctggcaacggtgtttcaaagcatgactttgatacaaagttagaaaagatgattgacttagtttacaatgaccaatgcacgccgggaaaccctcgccaaccaagcttggcagaaattcgtcaattgttgaaagatcagttttaa

SEQ ID NO: 15 - protein, 900 aa, Leuconostoc mesenteroides
MAEAIAKKPAKRVLTPEEKAELQTQAEKMTEVLIEKSQKALSEFSTFSQEQVDKIVAAMALAGSENSLLLAHAAHDETGRGVVEDKDTKNRFASESVYNAIKFDKTVGVISEDKIQGKVELAAPLGILAGIVPTTNPTSTTIFKSMLTAKTRNTIIFAFHPQAQKASVLAAKIVYDAAVKAGAPENFIQWIEKPSLYATSALIQNPHIASILATGGPSMVNAALKSGNPSMGVGAGNGAVYIDATVDTDRAVSDLLLSKRFDNGMICATENSAVIQAPIYDEILTKLQEQGAYLVPKKDYKKIADYVFKPNAEGFGIAGPVAGMSGRWIAEQAGVKIPDGKDVLLFELDQKNIGEALSSEKLSPLLSIYKVEKREEAIETVQSLLNYQGAGHNAAIQIGSQDDPFIKEYADAIGASRILVNQPDSIGGVGDIYTDAMRPSLTLGTGSWGKNSLSHNLSTYDLLNIKTVARRRNRPQWVRLPKEVYYEANAITYLQDLPTINRAFIVADPGMVQFGFVGRVLGQLELRQEQVETNIYGSVKPDPTLSQAVEIARQMADFKPDTVILLGGGSALDAGKIGRFLYEYSTRHEGILEDDEAIKDLFLELQQKFMDIRKRIVKFYHARLTQMVAIPTTSGTGSEVTPFAVITDDETHVKYPLADYELTPEVAIVDPEFVMTVPQHTVSWSGLDALSHALESYVSVMASEFTRPWALQAIKLIFDNLTNSYNYDPKHPTKEGQNARTKMHYASTLAGMSFANAFLGLNHSLAHKTGGEFGLPHGMAIAIAMPHVIKFNAVTGNVKRTPYPRYETYTAQKDYADIARYLGLKGETDAELVDVLIAEIKKLAASVGVNQTLSGNGVSKHDFDTKLEKMIDLVYNDQCTPGNPRQPSLAEIRQLLKDQF

SEQ ID NO: 16 - DNA, 2697 bp, Oenococcus oeni
  source 1..2697: mol_type = other DNA
atggcagttgaaacgaagaagattactaaaaagatactgacttccgacgaacttaaagacaaaaaggagacagcaaaaaaatacattggagaattagtcgataaatcaagacaggcattattagaattctctcaatatagtcaatcacaagttgacaaaattgttgccgcaatggctttagccggttccgaacattctttggaattagcacactttgctcgaaatgaaactggaagaggagttgttgaagacaaagatacgaaaaatcgttttgcctcggaatctgtatataacacgattaaaaatgacaaaacagttggcgtaattgatgaagacccaatcactggcaaagttcagttagctgcaccacttgggattttagccggaattgttccaacgacaaatccaacgtctaccacgatttttaaatcattgttaaccgctaagacacgtaatacgatcatttttgcttttcacccgcaagcacaaaaatcgtctgtagcggctgcgaaaattgtttaccaggcggctagaaaagctggagcaccgaaagatttcattcagtggatcgaaacgccttccttggaaaatacgactgctttgatgcaaaatcctgaaatagcttcaattttggctactggaggcccttctatggttcatgccgcattaacatcgggaaacccttcaatgggtgttggggcaggaaatggagctgttttcattgaccatacggctcgtgttcgtcgagcggcagaagacttgttgctatcaaaacgttttgataatggaatgatttgcgctacagagaattccgcagttatcgaagcttcggtttatgacgaatttatgaaattaatgcaggaaaagggtacttatctcgttccgaaggatgattacaaaaagatcgctgattttgttttcagcgataaacatgcagttaatggtccggttgccgggatgagtggccgttggattgctgaacatgccggtgttactttgccgaagggcaaagatgttttgttgtttgagttggatcaagacgacattggcgaaaaactttcctcggaaaaacttagccccttgctttcggtctataaagctgctgatcgtaaagaagcgataaaagttgttcaaagacttttgaactatcaaggagctggtcataatgcggcaatccagattggcgctcaagacgatccctttgttaaagaatatgccgatgcagtttctgcatctagaattttggttaaccagcccgattcaataggtggagtcggcgatatttatacagatgcattgcgtccaagtatgacgcttggtactggttcatgggggaagaattcattgtcacataatttatcaacttacgatttgttgaatgtcaaaacggttgcccgcagacgtaatcgtcctcaatgggttcgtcttccaaaagatatctactatgagaaaaatgccattacctatctgcaggaattaccgaacgtcaatcgggctttcgttattaccgatcattcgttggtcaaatatggattcgtcgatcggattcttgagcaattcgaattgcgtcccgaccaggttaaaacaagtatttacagttctgttcaaccggatccttatttgagtcaggctgttgaaattgcaaaacaaatgcaggagttcgaaccggatacagtaattgctcttggaggaggttcctctttggatgttgccaagatttcccgttttctttatgaatattctcaagaaccggaccacattggttttttggaaaacattgacgatattaaagaattgtttaaaggattgcagcaaaagtttatggatattcggaaacggattgttaaattcgaacatcagaatttgactcaactggttgctattccaacaacttcgggtaccggttcggaagtaacaccgttttcggttatcaccgatgatgaaactcacgttaaataccctttggccgattatgaattaactccgcaagttgcaatcgttgatcctgaattggttatgactgtaccaaaacggaccctggcttggtctggactggacgccttatctcactcgcttgaatcctacgtgtcggttatgagttctgaatttacacggccgtgggctttacaggcaatcaaactgatttttgaaaacatcgttgattcctataattacgatccaaagcacccaactaatcggggcgccgaggcacgtgaaaaaatgcattatgctgcaactttggccggcatgtctttcggaaacgccttcttgggtataaaccattcactggcccataaaaccggtggagagttcggtcttcctcatggtttggcaatcagcatcgcgatgccacatgtaattcgatttaatgcggttaccggaaacgtcaaacggactccttttcctcgttatgaggtttatacagcccaaaaagattatgccgatattgcgcgccatattggcctcagcggtaagaacgatgccgaattggttgaaaaacttatcgccaaaatcaaggaattaaccgatgccttggatgtcaatattactttgagcggcaatggagtcgataaaaagagctttgaacattctttggatcaactggttgatcttgtttacgatgaccagtgcacgccaggaaatccgcgacaaccgaatttagctgaaattaggcagttattgatcgaccaattttaa

SEQ ID NO: 17 - protein, 898 aa, Oenococcus oeni
MAVETKKITKKILTSDELKDKKETAKKYIGELVDKSRQALLEFSQYSQSQVDKIVAAMALAGSEHSLELAHFARNETGRGVVEDKDTKNRFASESVYNTIKNDKTVGVIDEDPITGKVQLAAPLGILAGIVPTTNPTSTTIFKSLLTAKTRNTIIFAFHPQAQKSSVAAAKIVYQAARKAGAPKDFIQWIETPSLENTTALMQNPEIASILATGGPSMVHAALTSGNPSMGVGAGNGAVFIDHTARVRRAAEDLLLSKRFDNGMICATENSAVIEASVYDEFMKLMQEKGTYLVPKDDYKKIADFVFSDKHAVNGPVAGMSGRWIAEHAGVTLPKGKDVLLFELDQDDIGEKLSSEKLSPLLSVYKAADRKEAIKVVQRLLNYQGAGHNAAIQIGAQDDPFVKEYADAVSASRILVNQPDSIGGVGDIYTDALRPSMTLGTGSWGKNSLSHNLSTYDLLNVKTVARRRNRPQWVRLPKDIYYEKNAITYLQELPNVNRAFVITDHSLVKYGFVDRILEQFELRPDQVKTSIYSSVQPDPYLSQAVEIAKQMQEFEPDTVIALGGGSSLDVAKISRFLYEYSQEPDHIGFLENIDDIKELFKGLQQKFMDIRKRIVKFEHQNLTQLVAIPTTSGTGSEVTPFSVITDDETHVKYPLADYELTPQVAIVDPELVMTVPKRTLAWSGLDALSHSLESYVSVMSSEFTRPWALQAIKLIFENIVDSYNYDPKHPTNRGAEAREKMHYAATLAGMSFGNAFLGINHSLAHKTGGEFGLPHGLAISIAMPHVIRFNAVTGNVKRTPFPRYEVYTAQKDYADIARHIGLSGKNDAELVEKLIAKIKELTDALDVNITLSGNGVDKKSFEHSLDQLVDLVYDDQCTPGNPRQPNLAEIRQLLIDQF

SEQ ID NO: 18 - DNA, 1476 bp, Saccharomyces cerevisiae
  source 1..1476: mol_type = other DNA
atggagagtcgaactacagggcctttaacgactgaaacctacgatggccccactgtggccttcatgatattaggtgccgccctagtattttttatggtgcccggattgggattcttgtactccggattggcaagaaggaagtctgcactagcactaatctgggttgtattaatggcgactttggtcggtatactgcaatggtatttctggggttactctctagctttttcaaagtccgctccgaataataaattcattgggaatctagattcgtttggctttagaaacgtgtacggaaaaaaattcgatgaagatgcctaccctgagctcgcgtatgcaaccttccaaatgatgttttcgtgcgtcaacttaagtattatcgctggcgccactgccgaaagaggcaggctgctaccgcacatggtttttctctttattctagctaccattggatattgtccagtgacgtattggatttggtcaccaggtggttgggcataccaatggggagtcctcgattgggcaggcggcggcaacattgaaatattaagcgctgtttccgggtttgtttactcttggtttttgggcaaaagaaatgaaaagttactgataaatttcaggcctcataatgtttcattggtcactctaggcacatccatactgtggtttggctggctgctatttaattctgcatcctcattatccccaaatttgaggtcagtttatgcattcatgaatacatgtctcagtgccattactggtgggatgacgtggtgtcttctggattacagatcggagaagaaatggtcgacagttggtctgtgctccggtatcatttctgggctggtggctgcaacgccaagctcaggctgtataaccctttacggttcacttattcaaggcattgtggcgggggtagtgtgtaactttgcgacgaagttgaaatactacgctaaagtagatgatgccatggacattctagctgagcacggggttgcaggcgtaataggactaattttcaatgccctttttggagcagactgggtcattggtatggatggcactacagagcacgagggcggctgggtaactcacaattacaagcaaatgtataagcagatcgcttacattgccgcatccattgggtacactgctgctgtaactgcaataatctgctttgtgctcggctacatacccggtatgaggctaagaatatcagaagaggcagaggaggcgggtatggacgaagatcaaattggcgaatttgcgtacgattatgtggaagtgagaagagattactatctatggggtgtagacgaagattcacaacgctctgatgtaaatcaccgggtgaacaacgctcatttggccgctgaacgtagcagtagcggtactaatagttcctcggatgggaatggagaaatgattcaatccgaaaagatcctaccaattcatcaagaagatcctgccaatagg

SEQ ID NO: 19 - protein, 492 aa, Saccharomyces cerevisiae
MESRTTGPLTTETYDGPTVAFMILGAALVFFMVPGLGFLYSGLARRKSALALIWVVLMATLVGILQWYFWGYSLAFSKSAPNNKFIGNLDSFGFRNVYGKKFDEDAYPELAYATFQMMFSCVNLSIIAGATAERGRLLPHMVFLFILATIGYCPVTYWIWSPGGWAYQWGVLDWAGGGNIEILSAVSGFVYSWFLGKRNEKLLINFRPHNVSLVTLGTSILWFGWLLFNSASSLSPNLRSVYAFMNTCLSAITGGMTWCLLDYRSEKKWSTVGLCSGIISGLVAATPSSGCITLYGSLIQGIVAGVVCNFATKLKYYAKVDDAMDILAEHGVAGVIGLIFNALFGADWVIGMDGTTEHEGGWVTHNYKQMYKQIAYIAASIGYTAAVTAIICFVLGYIPGMRLRISEEAEEAGMDEDQIGEFAYDYVEVRRDYYLWGVDEDSQRSDVNHRVNNAHLAAERSSSGTNSSSDGNGEMIQSEKILPIHQEDPANR

SEQ ID NO: 20 - DNA, 1497 bp, Saccharomyces cerevisiae
  source 1..1497: mol_type = other DNA
atgtcttacaattttacaggtacgcctacaggcgaaggaacgggtggtaactcgttgacaacagatttgaatacacaatttgacttggccaacatgggatggatcggtgtggcttcagcaggtgtgtggattatggtcccaggtatcggtttattatattctggtttatccaggaaaaagcatgctttatccttgctttgggcctcgatgatggcttccgccgtgtgtattttccaatggtttttctggggatactcattagctttctcacacaacactagaggtaacggttttattggtaccttggaattctttgggtttcgtaacgttttaggagccccatctagtgtcagttctcttcccgatatactgtttgccgtttaccaaggtatgtttgccgcagtcaccggtgccctaatgctaggtggtgcctgcgagagggcaaggttgtttcctatgatggtgttcttgtttttatggatgactattgtttattgtcctattgcatgctgggtctggaatgccgagggttggttggtcaaattgggtagcttggactatgcaggtggtttatgtgtccatttaacatctggacatggtggtctagtttacgctttgatactgggtaagcgtaatgaccctgttacacgtaaagggatgcccaagtacaaaccacattccgtcacctcggtggttttaggcacagtgttcttatggtttggttggatgttctttaacggaggctctgcaggtaatgcaactatacgagcatggtactctattatgtccacaaacttagctgctgcttgcggtggcttgacatggatggttatcgattatttcagatgcggaagaaagtggactacagttggtttgtgttcaggtatcatcgctggcctagtgggtatcaccccagccgccgggttcgtgccaatctggtcagccgttgtcattggtgtggttactggtgcaggatgtaaccttgctgttgacttaaagagtctattgcgcatcgatgatggtctagattgttactctatccatggtgtgggtggttgtattggttctgtattaactggtatctttgctgcagactatgtaaatgccactgcaggctcttacattagtccaattgatggtggctggatcaatcatcactataaacaagttggttatcaattagcaggtatatgcgctgcactagcctggactgttactgtcacatctatcttgcttctaactatgaatgccattccatttttaaaactaagattaagtgctgatgaggaagaattaggtaccgacgctgctcaaattggtgaatttacatacgaggaatccactgcttacatcccagaaccaatcagatctaaaacatcggcacaaatgccacctcctcatgaaaacattgatgataagattgtgggtaacacagacgcagaaaagaattctacgccttccgacgcttcttctactaagaacactgaccatatagta

SEQ ID NO: 21 - protein, 499 aa, Saccharomyces cerevisiae
MSYNFTGTPTGEGTGGNSLTTDLNTQFDLANMGWIGVASAGVWIMVPGIGLLYSGLSRKKHALSLLWASMMASAVCIFQWFFWGYSLAFSHNTRGNGFIGTLEFFGFRNVLGAPSSVSSLPDILFAVYQGMFAAVTGALMLGGACERARLFPMMVFLFLWMTIVYCPIACWVWNAEGWLVKLGSLDYAGGLCVHLTSGHGGLVYALILGKRNDPVTRKGMPKYKPHSVTSVVLGTVFLWFGWMFFNGGSAGNATIRAWYSIMSTNLAAACGGLTWMVIDYFRCGRKWTTVGLCSGIIAGLVGITPAAGFVPIWSAVVIGVVTGAGCNLAVDLKSLLRIDDGLDCYSIHGVGGCIGSVLTGIFAADYVNATAGSYISPIDGGWINHHYKQVGYQLAGICAALAWTVTVTSILLLTMNAIPFLKLRLSADEEELGTDAAQIGEFTYEESTAYIPEPIRSKTSAQMPPPHENIDDKIVGNTDAEKNSTPSDASSTKNTDHIV

SEQ ID NO: 22 - DNA, 1467 bp, Saccharomyces cerevisiae
  source 1..1467: mol_type = other DNA
atggctcggggtgacggacatctatggacagagacatatgatagttccacagtcgcttttatgattttaggtgccgccctggttttcttcatggtaccagggctgggctttcttttttccggtttagcaagaagaaaatctgctctggctttgatttgggtagtgataatggctaccttagtaggtatactgcaatggtatttttggggctattctttagcattctctaagactgcgacgaacaacaaatttatcggcaacttggattcatttgggtttagaaacgtctatggcaaaatttcggatgattccacgtatcctgaactgatttatgccattttccaaatgatgttcatgtgtgtcgcattgagtattatagctggtgccactgcggaaagaggtaagctttttccacatatggtttttctttttgtttttgcgactttggtttactgtcccatcacttattggatttgggccccaggtggctgggcctaccaatggggggtattagactgggctggcggtgggaatattgaaatcctaagtgctgtggctggtttcgtttattcttattttctaggaagaagaaaagaaaacctcctgatcaactttagaccacataatgtttccatggtgactttaggtacttctatactttggtttggttggttgcttttcaatgctgcaagctcactgtcaccaaatatgaggtccgtatatgcgttcatgaacacttgtctcagcgccaccacgggtggaatgacgtggtgtttattagattatcgatctgaaaaaaaatggtccactgttgggttatgctccggcattatctgtggtttagttgctgccacgcctagctcgggttgtattactctatatggctctttgatccaaggtataatagcgggtgttgtttgtaattttgcaacaaaaataaagtattatttaaaagtggatgattccttagatctattagctgaacacggtatcgccggtgtggtgggattgatttttaacgctctatttgcagctgattgggttattggaatggacggcacaacaaagcataagggtggttggttgacgcataactggaaacaaatgtatattcaaattgcctatatcggtgcctctgccggctattgtgctgtggtcacggccatcatttgcttcgtattaggtaaaattccgggtgtccatctaagagtcactgaggaagccgaagcattggggttggatgaagatcaaataggcgaattcgcttacgattacgtggaggttaggagagattattaccagtggggtgtagatacagatgcacttcatactacatgcaatggcgctaattctgcgtctgagacaaatcctactgaggacagccaaaactcctcattgtcatcagctacagtaagcggccaaaacgaaaaaagtaataatcctaaattgcatcacgcaaaagaagca

SEQ ID NO: 23 - protein, 489 aa, Saccharomyces cerevisiae
MARGDGHLWTETYDSSTVAFMILGAALVFFMVPGLGFLYSGLARRKSALALIWVVIMATLVGILQWYFWGYSLAFSKTATNNKFIGNLDSFGFRNVYGKISDDSTYPELIYAIFQMMFMCVALSIIAGATAERGKLFPHMVFLFVFATLVYCPITYWIWAPGGWAYQWGVLDWAGGGNIEILSAVAGFVYSYFLGRRKENLLINFRPHNVSMVTLGTSILWFGWLLFNAASSLSPNMRSVYAFMNTCLSATTGGMTWCLLDYRSEKKWSTVGLCSGIICGLVAATPSSGCITLYGSLIQGIIAGVVCNFATKIKYYLKVDDSLDLLAEHGIAGVVGLIFNALFAADWVIGMDGTTKHKGGWLTHNWKQMYIQIAYIGASAGYCAVVTAIICFVLGKIPGVHLRVTEEAEALGLDEDQIGEFAYDYVEVRRDYYQWGVDTDALHTTCNGANSASETNPTEDSQNSSLSSATVSGQNEKSNNPKLHHAKEA

SEQ ID NO: 24 - DNA, 1362 bp, Saccharomyces cerevisiae
  source 1..1362: mol_type = other DNA
atgtcagagccagaatttcaacaagcttacgaagaagttgtctcctctttggaagactctactcttttcgaacaacacccagaatacagaaaggttttgccaattgtttctgttccagaaagaatcatacaattcagagtcacctgggaaaatgacaagggtgaacaagaagttgctcaaggttacagagtgcaatataactccgccaagggtccatacaagggtggtctacgtttccatccttccgtgaacttgtctatcttgaaattcttgggtttcgaacaaatcttcaagaactccttgaccggcctagacatgggtggtggtaaaggtggtctatgtgtggacttgaagggaagatctaataacgaaatcagaagaatctgttacgctttcatgagagaattgagcagacacattggtcaagacactgacgtgccagctggtgatatcggtgttggtggtcgtgaaattggttacctgttcggtgcttacagatcatacaagaactcctgggaaggtgtcttaaccggtaagggtttgaactggggtggttctttgatcagaccagaagccactggttacggtttagtttactatacccaagctatgatcgactatgccacaaacggtaaggaatctttcgaaggtaagcgcgtcaccatctctggtagtggtaacgttgctcaatacgctgccttgaaggttattgagctaggtggtactgtcgtttccctatctgactccaagggttgtatcatctctgaaactggtatcacctccgaacaagtcgctgatatttccagtgctaaggtcaacttcaagtccttggaacaaatcgtcaacgaatactctactttctccgaaaacaaagtgcaatacattgctggtgctcgtccatggacccacgtccaaaaggtcgacattgctttgccatgtgccacccaaaatgaagtcagcggtgaagaagccaaggccttggttgctcaaggtgtcaagtttattgccgaaggttccaacatgggttccactccagaagctattgccgtctttgaaactgctcgttccaccgccactggaccaagcgaagctgtttggtacggtccaccaaaggctgctaacttgggtggtgttgctgtttctggtttagaaatggcacaaaactctcaaagaatcacatggactagcgaaagagttgaccaagagttgaagagaattatgatcaactgtttcaatgaatgtatcgactatgccaagaagtacactaaggacggtaaggtcttgccatctttggtcaaaggtgctaatatcgcaagtttcatcaaggtctctgatgctatgtttgaccaaggtgatgtattt

SEQ ID NO: 25 - protein, 454 aa, Saccharomyces cerevisiae
MSEPEFQQAYEEVVSSLEDSTLFEQHPEYRKVLPIVSVPERIIQFRVTWENDKGEQEVAQGYRVQYNSAKGPYKGGLRFHPSVNLSILKFLGFEQIFKNSLTGLDMGGGKGGLCVDLKGRSNNEIRRICYAFMRELSRHIGQDTDVPAGDIGVGGREIGYLFGAYRSYKNSWEGVLTGKGLNWGGSLIRPEATGYGLVYYTQAMIDYATNGKESFEGKRVTISGSGNVAQYAALKVIELGGTVVSLSDSKGCIISETGITSEQVADISSAKVNFKSLEQIVNEYSTFSENKVQYIAGARPWTHVQKVDIALPCATQNEVSGEEAKALVAQGVKFIAEGSNMGSTPEAIAVFETARSTATGPSEAVWYGPPKAANLGGVAVSGLEMAQNSQRITWTSERVDQELKRIMINCFNECIDYAKKYTKDGKVLPSLVKGANIASFIKVSDAMFDQGDVF

SEQ ID NO: 26 - DNA, 3276 bp, Saccharomyces cerevisiae
  source 1..3276: mol_type = other DNA
atgctttttgataacaaaaatcgcggtgctttaaactcactgaacacaccagatattgcttctttatcaatatcatccatgtcggactatcacgtgtttgattttcccggtaaggacctgcagagagaggaagtgatagatttgctagatcagcaagggtttattcccgacgatttgatcgaacaagaagtagattggttttataactcattgggtattgacgatttgttcttctcgagagaatctccccaattaatctcgaatatcatacattctttgtatgcttcaaagctagatttctttgcgaagtccaaattcaacggaattcagccaaggctattcagcattaaaaacaaaattataactaatgataatcatgccatctttatggaatctaatactggtgtcagcataagcgattctcagcaaaaaaactttaaatttgctagtgacgccgtcggaaacgatactttggagcatggtaaggataccatcaaaaaaaataggattgaaatggatgattcttgtccaccttatgaattagattccgaaattgatgaccttttcctggataacaagtctcaaaaaaactgcagattagtttctttttgggctccagaaagcgaattaaagctaacttttgtttatgagagtgtttaccctaatgatgatccagccggcgtagatatttcctctcaggatttgctgaaaggtgatattgaatcgattagtgataagaccatgtacaaagtttcgtcgaacgaaaataaaaaactatacggtctcttacttaagttggttaaagaaagagaaggtcctgtcattaagactactcgctccgtagaaaataaggatgaaattaggttattagtcgcttacaagcgattcaccactaagcgttattactctgctttgaactctttgttccactattacaagttgaaaccttctaagttctatttagagtcgtttaatgttaaggatgatgacatcattatcttttccgtttatttgaacgagaaccagcaattggaagatgttctacttcacgatgtggaggcagcattgaaacaggttgaaagagaagcttcattgctatacgctatcccaaacaattctttccatgaggtttaccagagacgtcaattctcgcccaaagaagctatatatgctcatattggtgctatattcattaaccattttgttaatcgtttaggctctgattatcaaaaccttttatctcaaatcaccattaagcgtaatgatactactcttttggagattgtagaaaacctaaaaagaaagttaagaaatgaaaccttaactcagcaaactattatcaacatcatgtcgaagcattacactataatttccaagttgtataaaaattttgctcaaattcactattatcataatagtactaaagatatggagaagacattatcttttcaaagactggaaaaagtggagccttttaagaatgaccaagagttcgaagcttacttgaataaattcattccaaatgattcacctgatttgttgatcctgaaaacactgaacatcttcaacaagtctattttgaagacaaatttctttattacaagaaaagtagcaatatcattcagattagatccttccctggtgatgacaaaattcgaatatccagagacaccctatggtatattttttgtcgttggtaatactttcaaagggttccatatcaggttcagagatatcgcaaggggcggtattcgtatagtctgttccaggaatcaggatatttatgatttgaattccaagaacgttattgatgagaactatcaattggcctctactcagcaacgtaaaaataaggatattccagagggtggctctaaaggtgtcatcttattgaacccaggattggtagaacatgaccagacatttgtcgccttttcccaatatgtggatgcaatgattgacattctaatcaacgatccattaaaggaaaactatgtcaaccttttaccaaaggaggaaatattattttttggcccagatgaaggaactgctggtttcgtggattgggcaactaaccatgctcgtgtgaggaactgcccatggtggaaatcatttttgactggaaaatccccatctttgggtggtattccccatgacgaatatggtatgacttctctgggtgttcgtgcttatgttaataaaatttacgaaactttaaacttgacaaattctactgtttacaaattccaaactggtggtccggatggtgatttgggatccaatgaaattcttttatcttcgccaaacgaatgttatttggcaattctggacggttcaggtgtcctgtgtgatcctaaaggtttagataaagatgaattatgccgcttggcacatgaaaggaaaatgatttccgatttcgacacttccaaattatcaaacaacggattttttgtttctgtggatgcaatggatatcatgctaccaaatggtacaattgtagctaacggcacaaccttcagaaacacctttcatactcaaattttcaaatttgtggatcatgtcgacatttttgttccatgcggtggtagaccaaactcaattactctaaataatctacattattttgttgacgaaaagactgggaaatgtaaaattccatatattgtggagggtgccaatctatttataacgcaacctgctaaaaatgctttggaggaacatggctgtattctgttcaaagatgcttctgcaaacaaaggtggtgtcacatcttcatcaatggaagtgttggcctcactagcgcttaacgataacgacttcgtgcacaaatttattggagatgttagtggtgagaggtctgcgttgtacaagtcgtacgttgtagaagtgcagtcaagaattcagaaaaatgctgaattagagtttggtcagttatggaatttgaatcaactaaatggaacccacatttcagaaatttcaaaccaattgtccttcactataaacaaattgaacgacgatctagttgcttctcaagagttgtggctcaatgatctaaaattaagaaactacctattgttggataaaataattccaaaaattctgattgatgttgctgggcctcagtccgtattggaaaacattccagagagctatttgaaagttcttctgtcgagttacttatcaagcacttttgtttaccagaacggtatcgatgttaacattggaaaattcttggaatttattggtgggttaaaaagagaagcggaggcaagtgct

SEQ ID NO: 27 - protein, 1092 aa, Saccharomyces cerevisiae
MLFDNKNRGALNSLNTPDIASLSISSMSDYHVFDFPGKDLQREEVIDLLDQQGFIPDDLIEQEVDWFYNSLGIDDLFFSRESPQLISNIIHSLYASKLDFFAKSKFNGIQPRLFSIKNKIITNDNHAIFMESNTGVSISDSQQKNFKFASDAVGNDTLEHGKDTIKKNRIEMDDSCPPYELDSEIDDLFLDNKSQKNCRLVSFWAPESELKLTFVYESVYPNDDPAGVDISSQDLLKGDIESISDKTMYKVSSNENKKLYGLLLKLVKEREGPVIKTTRSVENKDEIRLLVAYKRFTTKRYYSALNSLFHYYKLKPSKFYLESFNVKDDDIIIFSVYLNENQQLEDVLLHDVEAALKQVEREASLLYAIPNNSFHEVYQRRQFSPKEAIYAHIGAIFINHFVNRLGSDYQNLLSQITIKRNDTTLLEIVENLKRKLRNETLTQQTIINIMSKHYTIISKLYKNFAQIHYYHNSTKDMEKTLSFQRLEKVEPFKNDQEFEAYLNKFIPNDSPDLLILKTLNIFNKSILKTNFFITRKVAISFRLDPSLVMTKFEYPETPYGIFFVVGNTFKGFHIRFRDIARGGIRIVCSRNQDIYDLNSKNVIDENYQLASTQQRKNKDIPEGGSKGVILLNPGLVEHDQTFVAFSQYVDAMIDILINDPLKENYVNLLPKEEILFFGPDEGTAGFVDWATNHARVRNCPWWKSFLTGKSPSLGGIPHDEYGMTSLGVRAYVNKIYETLNLTNSTVYKFQTGGPDGDLGSNEILLSSPNECYLAILDGSGVLCDPKGLDKDELCRLAHERKMISDFDTSKLSNNGFFVSVDAMDIMLPNGTIVANGTTFRNTFHTQIFKFVDHVDIFVPCGGRPNSITLNNLHYFVDEKTGKCKIPYIVEGANLFITQPAKNALEEHGCILFKDASANKGGVTSSSMEVLASLALNDNDFVHKFIGDVSGERSALYKSYVVEVQSRIQKNAELEFGQLWNLNQLNGTHISEISNQLSFTINKLNDDLVASQELWLNDLKLRNYLLLDKIIPKILIDVAGPQSVLENIPESYLKVLLSSYLSSTFVYQNGIDVNIGKFLEFIGGLKREAEASA

SEQ ID NO: 28 - DNA, 3153 bp, Neurospora crassa
  source 1..3153: mol_type = other DNA
atggattctccttcagccccggtcccagcccacaagttggttgatcgcctcaaagatcagacgcccagacatccttctcctcagccgacacatgtcagctatcccaaggtgaacggcaacgggcacagggtgctccgctccgcaactgttggatatgttgcccctgtcttccagggaaaggcagagcagatgaagcaagtcaagaatatcattgtccagggcggctggattcccgagactctggttgacggtcagattgcgtggttctataatgagcttggcatcgacgacgtctacttccaactagaaaaccctcaggcggttgcaaatcatatcacatctctctatgcggccaaagttgctgctttctctcgcgaggacaagcgggaagagatcagactggatatggaagcctcggaccacgccatttacatcgacacaagcgaacccggcatgacctccttcgacggtccgcgatatgagcacagactcgagtccaagtacttggatggcgacgatacttcgaagcgtttccgtgtcgagactttcagatcccccggtgtcctggggcagaaggagaactccaaggcagctcttcgttgctactttgtctatcagtgtctctttgttgattctaacgccgatcccaaggaaactcgcctggaagtgatcagcgaccgcatgttccttgccaaggctaccaagaacaccaagcagatctaccaggacatcatccaggttgccgtgagccgacatggtcccgtcattgaggttttcgacatcgagggctcggaggaaatgagactggtagttgccttccgctctcgaacagcaaagggtatcttcagcgccctcagcgacctttaccactactacggcgtcacaagctctcgtaagtacgttgagcagttctccaatggtattaccgtcatgagtatctatctgcgccctgctgcgaacatcgacggcaagcaccctcctcttgagcaatctattcaccagatcaccaaggagatttccttgctgtactgccttccccagaacaagtttcacaacatgtttgcctctggtgaactcagtctgcaggagaccatctatgcccactgcgtctgggtgtttgttcagcatttcttgaaccgtcttgggacagagtacacttcgctcattgcagccctggaccccaagaacaactcgcatgttgagattctctccaagatgaagaagcgtctgcgtaccgagacctttacacctgactacattcttgaaatcatcagctcccacccgcaacttgtgcgcgctctctacgcctcattcgccagcgttcaccttcgggtcggcagcgattatgatcgccatctcattgcgcctacgccggtcatggaggtcctttctgatgccaggcttaaggagaagatcaccaaggacgtttctaacgagcacgaggaaatggttatgactgccttccgtgtattcaacaatgcagtcctcaagacgaacttcttcactcccaccaaagtcgctttgagcttccggcttaatccctcgttccttccggaagtcgagtatcccaagccgctgtacggaatgttcctcgtcatcacttccgagtcgcgaggtttccacctccgcttcaaggatattgcacggggtggtattcgtattgtcaagtctcgaagtaaggaggcttaccagatcaatgcccgcaatctctttgatgagaactacggtcttgccagcacccagcagcgcaagaacaaggatattccggagggcggatcgaagggtgtcattctccttgaccccaagcagcaagacaggcaccgcgaggcctttgagaagtacatcgatagtatccttgaccttcttctgaaggctgagacgcctggcatcaagaaccccattgtcgacctctacggcaaggaggagattttgttcatgggaccggatgagaacactgctgaccttgtcgactgggccactgagcatgcccgggctcgcggcgctccttggtggaaatccttcttcactggtaaatcaccacgtcttggtggcatccctcacgactcctatggcatgacaaccttgtcggttcgtgagtatgtcaagggtatttaccgcaagcttgagcttgatccctccaagatcagaaagatgcagactggtggtcccgatggtgatctgggaagcaacgagattctgctcagcaacgagacttacactgctatcgttgacggatccggtgtcctctgcgaccccaacggcattgacaaggatgagttacgccgtcttgccaaggctcgtgctatgatctcaaactttgatatcgccaagctttccaaggatggttatcgtgtgctttgtgacgataccaacgtcacactccccaacggagaggttgtgcacaatggcactgcattccgtaacacgtaccatcttcgcgacaacggcatcactgatatgttcgtgccttgcggtggccgtcccgagtcgatcgacctctcctcagtcaacaagcttatcaaggatggaaagtcaaccatcccatatattgttgagggtgccaacctgttcatcactcaagatgccaagctcagactggaggaagccggctgcatcgtttacaaggatgccagtgccaacaaaggcggtgtaaccagctcatcgctcgaggtccttgcctccctcagctttgacgacaagggcttcgttacccacatgtgccacgattcccgcggcaatgcgcccgagttctaccaggcctatgtcaaggaagtgcaaaacaagatccaggacaatgcccgcctcgagttcgaggccatctggcgggaacatgagcagactggccttcctcgatctgttctctctgacaagttgtcattggccatcacctctcttgacgaggatctccagcgttcggagctctgggacaacgagaagattcgcaggtccgtccttgccgatgccttgcccaaccttctcattaacaagatcggcctcgataccatcatcgagcgtgttcccgattcgtatctgagggccatctttggcagctacctcgccagtcgcttcgtctatgagtttggtagctcgcccagccagtttgctttctatgatttcatgtcaaagagaatgggaaacatcaataaggagtaa

SEQ ID NO: 29 - protein, 454 aa, Neurospora crassa
MSNLPSEPEFEQAYKELAYTLENSSLFQKHPEYRTALTVASIPERVIQFRVVWEDDNGNVQVNRGYRVQFNSALGPYKGGLRLHPSVNLSILKFLGFEQIFKNALTGLSMGGGKGGADFDPKGKSDAEIRRFCCAFMAELHKHIGADTDVPAGDIGVGGREIGYMFGAYRKAANRFEGVLTGKGLSWGGSLIRPEATGYGLVYYVGHMLEYSGAGSYAGKRVALSGSGNVAQYAALKLIELGATVVSLSDSKGALVATGESGITVEDINAVMAIKEARQSLTSFQHAGHLKWIEGARPWLHVGKVDIALPCATQNEVSKEEAEGLLAAGCKFVAEGSNMGCTLEAIEVFENNRKEKKGEAVWYAPGKAANCGGVAVSGLEMAQNSQRLNWTQAEVDEKLKDIMKNAFFNGLNTAKTYVEAAEGELPSLVAGSNIAGFVKVAQAMHDQGDWWSKN

SEQ ID NO: 30 - DNA, 1371 bp, Saccharomyces cerevisiae
  source 1..1371: mol_type = other DNA
atgacaagcgaaccagagtttcagcaggcttacgatgagatcgtttcttctgtggaggattccaaaatttttgaaaaattcccacagtataaaaaagtgttacctattgtttctgtcccggagaggatcattcaattcagggtcacgtgggaaaatgataatggcgagcaagaagtggctcaaggatacagggtgcagttcaattcagccaagggcccttacaagggtggcctacgcttccacccatcagtgaacctgtctatcctaaaatttttgggttttgaacagatcttcaagaatgcgctcactgggctagatatgggcggtggtaagggtggcctgtgtgtggacttgaaaggcaagtctgacaacgagatcagaaggatttgttatgcgttcatgagagaactgagcaggcatattggtaaggacacagacgtgcccgcaggagatattggtgtcggtggccgtgaaattggctacctattcggcgcttacagatcatacaagaactcctgggaaggtgtgttgactggtaagggtttaaactggggtggctcacttatcaggccggaggccaccgggttcggcttagtttactatacgcaagcaatgatcgattatgcaacaaacggcaaggagtcgtttgagggcaaacgtgtgacaatctccggaagtggcaatgttgcgcaatatgcagctttgaaagtgatcgagctgggtggtattgtggtgtctttatccgattcgaaggggtgcatcatctctgagacgggcattacttctgagcaaattcacgatatcgcttccgccaagatccgtttcaagtcgttagaggaaatcgttgatgaatactctactttcagcgaaagtaagatgaagtacgttgcaggagcacgcccatggacgcatgtgagcaacgtcgacattgccttgccctgtgccacccaaaacgaggtcagtggtgacgaagccaaggccctagtggcatctggcgttaagttcgttgccgaaggtgctaacatgggttctacacccgaggctatttctgttttcgaaacagcgcgtagcactgcaaccaatgcaaaggatgcagtttggtttgggccaccaaaggcagctaacctgggcggcgtggcagtatccggtctggaaatggctcagaattctcaaaaagtaacttggactgccgagcgggtcgatcaagaactaaagaagataatgatcaactgcttcaacgactgcatacaggccgcacaagagtactctacggaaaaaaatacaaacaccttgccatcattggtcaagggggccaacattgccagcttcgtcatggtggctgacgcaatgcttgaccagggagacgttttt

SEQ ID NO: 31 - protein, 457 aa, Saccharomyces cerevisiae
MTSEPEFQQAYDEIVSSVEDSKIFEKFPQYKKVLPIVSVPERIIQFRVTWENDNGEQEVAQGYRVQFNSAKGPYKGGLRFHPSVNLSILKFLGFEQIFKNALTGLDMGGGKGGLCVDLKGKSDNEIRRICYAFMRELSRHIGKDTDVPAGDIGVGGREIGYLFGAYRSYKNSWEGVLTGKGLNWGGSLIRPEATGFGLVYYTQAMIDYATNGKESFEGKRVTISGSGNVAQYAALKVIELGGIVVSLSDSKGCIISETGITSEQIHDIASAKIRFKSLEEIVDEYSTFSESKMKYVAGARPWTHVSNVDIALPCATQNEVSGDEAKALVASGVKFVAEGANMGSTPEAISVFETARSTATNAKDAVWFGPPKAANLGGVAVSGLEMAQNSQKVTWTAERVDQELKKIMINCFNDCIQAAQEYSTEKNTNTLPSLVKGANIASFVMVADAMLDQGDVF

SEQ ID NO: 32 - DNA, 6435 bp, Saccharomyces cerevisiae
  source 1..6435: mol_type = other DNA
atgccagtgttgaaatcagacaatttcgatccattggaagaagcttacgaaggtgggacaattcaaaactataacgatgaacaccatcttcataaatcttgggcaaatgtgattccggacaaacgaggactttacgaccctgattatgaacatgacgcttgtggtgtcggtttcgtagcaaataagcatggtgaacagtctcacaagattgttactgacgctagatatcttttagtgaatatgacacatcgtggtgccgtctcatctgatgggaacggtgacggtgccggtattctgctaggtattcctcacgaatttatgaaaagagaattcaagttagatcttgatctagacatacctgagatgggcaaatacgccgtaggtaacgtcttcttcaagaagaacgaaaaaaataacaagaaaaatttaattaagtgtcagaagattttcgaggatttagctgcatccttcaacttatccgtattaggttggagaaacgtccccgtagattctactattttaggagacgttgcattatctcgtgaacctactattctacagccattattggttccattgtatgatgaaaaacaaccggagtttaatgaaactaaatttagaactcaattgtatcttttaaggaaggaggcctctcttcaaataggactggaaaactggttctatgtttgttccctaaacaataccaccattgtttacaagggtcaattgacgccagctcaagtgtataactactatcccgacttgactaatgcgcatttcaaatcccacatggcgttggtccattcaagattttccactaatactttcccctcttgggatagagctcaacctttacgttggctagctcataatggtgaaattaacaccttaagaggtaacaagaattggatgcgctccagagaaggtgtgatgaattcagcaactttcaaagatgagttagacaaactatacccaattatcgaagaaggtggttctgattcagctgcattggataacgttttagaactattgactattaatggcacattatctctacctgaagctgttatgatgatggttcctgaagcgtatcataaggatatggattctgacctaaaagcatggtacgactgggctgcatgtctgatggaaccttgggatggtccagctttgttaactttcactgatggacgttactgtggtgctatattggatagaaatggtttaagaccttgtcgttattacatcactagtgatgacagagttatctgtgcttcagaggtaggtgtcattcctatcgaaaattcattggttgttcaaaaaggtaaactgaagccaggtgatttattcctagtggatactcaattgggtgaaatggtcgatactaaaaagttaaaatctcaaatctcaaaaagacaagattttaagtcttggttatccaaagtcatcaagttagacgacttgttatcaaaaaccgctaatttggttcctaaagaatttatatcacaggattcattgtctttgaaagttcaaagtgacccacgtctattggccaatggttataccttcgaacaagtcacatttctgttaactccaatggctttaacaggtaaagaagctttaggttcgatgggtaacgatgcgccactggcttgtttaaatgaaaatcctgtcttactttatgattatttcagacaattgtttgctcaagtgaccaatcctccaattgacccaattcgtgaagcaaatgttatgtcgttagaatgttatgtcggacctcaaggcaaccttttggaaatgcattcatctcaatgtgatcgtttattattgaaatctcctattttgcattggaatgagttccaagctttgaaaaacattgaagctgcttacccatcatggtctgtagcagaaattgatatcacattcgacaagagtgagggtctattgggctataccgacacaattgataaaatcactaagttagcgagcgaagcaattgatgatggtaaaaagatcttaataattactgacaggaaaatgggtgccaaccgtgtttccatctcctctttgattgcaatttcatgtattcatcatcacctaatcagaaacaagcagcgttcccaagttgctttgattttggaaacaggtgaagccagagaaattcaccatttctgtgtcctactaggttatggttgtgatggtgtttatccatacttagccatggaaactttggtcagaatgaatagagaaggtctacttcgtaatgtcaacaatgacaatgatacacttgaggaagggcaaatactagaaaattacaagcacgctattgatgcaggtatcttgaaggttatgtctaaaatgggtatctccactctagcatcctacaaaggtgctcaaatttttgaagccctaggtttagataactctattgttgatttgtgtttcacaggtacttcttccagaattagaggtgtaactttcgagtatttggctcaagatgccttttctttacatgagcgtggttatccatccagacaaaccattagtaaatctgttaacttaccagaaagtggtgaataccactttagggatggtggttacaaacacgtcaacgaaccaaccgcaattgcttcgttacaagatactgtcagaaacaaaaatgatgtctcttggcaattatatgtaaagaaggaaatggaagcaattagagactgtacactaagaggactgttagaattagattttgaaaattctgtcagtatccctctagaacaagttgaaccatggactgaaattgccagaagatttgcgtcaggtgcaatgtcttatggttctatttctatggaagctcactctacattggctattgccatgaatcgtttaggggccaaatccaattgtggtgaaggtggtgaagacgcagaacgttctgctgttcaagaaaacggtgatactatgagatctgctatcaaacaagttgcttccgctagattcggtgtaacttcatactacttgtcagatgctgatgaaatccaaattaagattgctcagggtgctaagccgggtgaaggtggtgaactaccagcccacaaagtgtctaaggatatcgcaaaaaccaggcactccacccctaatgttgggttaatctctcctcctcctcatcacgatatttattccattgaagatttgaaacaactgatttatgatttgaaatgtgctaatccaagagcgggaatttctgtaaagttggtttccgaagttggtgttggtattgttgcctctggtgtagctaaggctaaagccgatcatatcttagtttctggtcatgatggtggtacaggtgctgcaagatggacgagtgtcaaatatgcgggtttgccatgggaattaggtctagctgaaactcaccagactttagtcttgaatgatttaagacgtaatgttgttgtccaaaccgatggtcaattgagaactgggtttgatattgctgttgcagttttattaggggcagaatcttttaccttggcaacagttccattaattgctatgggttgtgttatgttaagaagatgtcacttgaactcttgtgctgttggtattgccacacaagatccatatttgagaagtaagtttaagggtcagcccgaacatgttatcaacttcttctattacttgatccaagatttaagacaaatcatggccaagttaggattccgtaccattgacgaaatggtgggtcattctgaaaaattaaagaaaagggacgacgtaaatgccaaagccataaatatcgatttatctcctattttgaccccagcacatgttattcgtccaggtgttccaaccaagttcactaagaaacaagaccacaaactccacacccgtctagataataagttaatcgatgaggctgaagttactttggatcgtggcttaccagtgaatattgacgcctctataatcaatactgatcgtgcactcggttctactttatcttacagagtctcgaagaaatttggtgaagatggtttgccaaaggacaccgttgtcgttaacatagaaggttcagcgggtcaatcttttggtgctttcctagcttctggtatcacttttatcttgaatggtgatgctaatgattatgttggtaaaggtttatccggtggtattattgtcattaaaccaccaaaggattctaaattcaagagtgatgaaaatgtaattgttggtaacacttgtttctatggtgctacttctggtactgcattcatttcaggtagtgccggtgagcgtttcggtgtcagaaactctggtgccaccatcgttgttgagagaattaagggtaacaatgcctttgagtatatgactggtggtcgtgccattgtcttatcacaaatggaatccctaaacgccttctctggtgctactggtggtattgcatactgtttaacttccgattacgacgattttgttggaaagattaacaaagatactgttgagttagaatcattatgtgacccggtcgagattgcgtttgttaagaatttgatccaggagcattggaactacacacaatctgatctagcagccaggattctcggtaatttcaaccattatttgaaagatttcgttaaagtcattccaactgattataagaaagttttgttgaaggagaaagcagaagctgccaaggcaaaggctaaggcaacttcagaatacttaaagaagtttagatcgaaccaagaagttgatgacgaagtcaatactctattgattgctaatcaaaaagctaaagagcaagaaaaaaagaagagtattactatttcaaataaggccactttgaaggagcctaaggttgttgatttagaagatgcagttccagattccaaacagctagagaagaatagcgaaaggattgaaaaaacacgtggttttatgatccacaaacgtcgtcatgagacacacagagatccaagaaccagagttaatgactggaaagaatttactaaccctattaccaagaaggatgccaaatatcaaactgcgagatgtatggattgtggtacaccattctgtttatctgataccggttgtcccctatctaacattatccccaagtttaatgaattgttattcaagaaccaatggaagttggcactggacaaattgctagagacaaacaatttcccagaattcactggaagagtatgtccagcaccctgtgagggagcttgtacactaggtattattgaagacccagtcggcataaaatcggttgaaagaattatcattgacaatgctttcaaggaaggatggattaagccttgtccaccaagtacacgcactggctttacagtgggtgtcattggttctggtccagcaggtttagcgtgtgctgatatgttgaaccgtgccggacatacggtcactgtttatgaaagatccgaccgttgtggtgggttattgatgtatggtattccaaacatgaagttggataaggctatagtgcaacgtcgtattgatctattgagtgccgaaggtattgactttgttaccaacaccgaaattggtaaaaccataagcatggatgagctaaagaacaagcacaatgcagtagtgtatgctatcggttctaccattccacgtgacttacctattaagggtcgtgaattgaagaatattgattttgccatgcagttgttggaatctaacacaaaagctttattgaacaaagatctggaaatcattcgtgaaaagatccaaggtaagaaagtaattgttgtcggtggtggtgacacaggtaacgattgtttaggtacatctgtaagacacggtgcagcatcagttttgaatttcgaattgttgcctgagccaccagtggaacgtgccaaagacaatccatggcctcaatggccgcgtgtcatgagagtggactacggtcatgctgaagtgaaagagcattatggtagagaccctcgtgaatactgcatcttgtccaaggaatttatcggtaacgatgagggtgaagtcactgccatcagaactgtgcgcgtagaatggaagaagtcacaaagtggcgtatggcaaatggtagaaattcccaacagtgaagagatctttgaagccgatatcattttgttgtctatgggtttcgtgggtcctgaattgatcaatggcaacgataacgaagttaagaagacaagacgtggtacgattgccacactcgacgactcctcatactctattgatggaggaaagacttttgcatgtggtgactgtagaagagggcaatctttgattgtctgggccatccaagaaggtagaaaatgtgctgcctctgtcgataagttcctaatggacggcactacgtatctaccaagtaatggtggtatcgttcaacgtgattacaaactattgaaagaattagctagtcaagtc

SEQ ID NO: 33 - protein, 2145 aa, Saccharomyces cerevisiae
MPVLKSDNFDPLEEAYEGGTIQNYNDEHHLHKSWANVIPDKRGLYDPDYEHDACGVGFVANKHGEQSHKIVTDARYLLVNMTHRGAVSSDGNGDGAGILLGIPHEFMKREFKLDLDLDIPEMGKYAVGNVFFKKNEKNNKKNLIKCQKIFEDLAASFNLSVLGWRNVPVDSTILGDVALSREPTILQPLLVPLYDEKQPEFNETKFRTQLYLLRKEASLQIGLENWFYVCSLNNTTIVYKGQLTPAQVYNYYPDLTNAHFKSHMALVHSRFSTNTFPSWDRAQPLRWLAHNGEINTLRGNKNWMRSREGVMNSATFKDELDKLYPIIEEGGSDSAALDNVLELLTINGTLSLPEAVMMMVPEAYHKDMDSDLKAWYDWAACLMEPWDGPALLTFTDGRYCGAILDRNGLRPCRYYITSDDRVICASEVGVIPIENSLVVQKGKLKPGDLFLVDTQLGEMVDTKKLKSQISKRQDFKSWLSKVIKLDDLLSKTANLVPKEFISQDSLSLKVQSDPRLLANGYTFEQVTFLLTPMALTGKEALGSMGNDAPLACLNENPVLLYDYFRQLFAQVTNPPIDPIREANVMSLECYVGPQGNLLEMHSSQCDRLLLKSPILHWNEFQALKNIEAAYPSWSVAEIDITFDKSEGLLGYTDTIDKITKLASEAIDDGKKILIITDRKMGANRVSISSLIAISCIHHHLIRNKQRSQVALILETGEAREIHHFCVLLGYGCDGVYPYLAMETLVRMNREGLLRNVNNDNDTLEEGQILENYKHAIDAGILKVMSKMGISTLASYKGAQIFEALGLDNSIVDLCFTGTSSRIRGVTFEYLAQDAFSLHERGYPSRQTISKSVNLPESGEYHFRDGGYKHVNEPTAIASLQDTVRNKNDVSWQLYVKKEMEAIRDCTLRGLLELDFENSVSIPLEQVEPWTEIARRFASGAMSYGSISMEAHSTLAIAMNRLGAKSNCGEGGEDAERSAVQENGDTMRSAIKQVASARFGVTSYYLSDADEIQIKIAQGAKPGEGGELPAHKVSKDIAKTRHSTPNVGLISPPPHHDIYSIEDLKQLIYDLKCANPRAGISVKLVSEVGVGIVASGVAKAKADHILVSGHDGGTGAARWTSVKYAGLPWELGLAETHQTLVLNDLRRNVVVQTDGQLRTGFDIAVAVLLGAESFTLATVPLIAMGCVMLRRCHLNSCAVGIATQDPYLRSKFKGQPEHVINFFYYLIQDLRQIMAKLGFRTIDEMVGHSEKLKKRDDVNAKAINIDLSPILTPAHVIRPGVPTKFTKKQDHKLHTRLDNKLIDEAEVTLDRGLPVNIDASIINTDRALGSTLSYRVSKKFGEDGLPKDTVVVNIEGSAGQSFGAFLASGITFILNGDANDYVGKGLSGGIIVIKPPKDSKFKSDENVIVGNTCFYGATSGTAFISGSAGERFGVRNSGATIVVERIKGNNAFEYMTGGRAIVLSQMESLNAFSGATGGIAYCLTSDYDDFVGKINKDTVELESLCDPVEIAFVKNLIQEHWNYTQSDLAARILGNFNHYLKDFVKVIPTDYKKVLLKEKAEAAKAKAKATSEYLKKFRSNQEVDDEVNTLLIANQKAKEQEKKKSITISNKATLKEPKVVDLEDAVPDSKQLEKNSERIEKTRGFMIHKRRHETHRDPRTRVNDWKEFTNPITKKDAKYQTARCMDCGTPFCLSDTGCPLSNIIPKFNELLFKNQWKLALDKLLETNNFPEFTGRVCPAPCEGACTLGIIEDPVGIKSVERIIIDNAFKEGWIKPCPPSTRTGFTVGVIGSGPAGLACADMLNRAGHTVTVYERSDRCGGLLMYGIPNMKLDKAIVQRRIDLLSAEGIDFVTNTEIGKTISMDELKNKHNAVVYAIGSTIPRDLPIKGRELKNIDFAMQLLESNTKALLNKDLEIIREKIQGKKVIVVGGGDTGNDCLGTSVRHGAASVLNFELLPEPPVERAKDNPWPQWPRVMRVDYGHAEVKEHYGRDPREYCILSKEFIGNDEGEVTAIRTVRVEWKKSQSGVWQMVEIPNSEEIFEADIILLSMGFVGPELINGNDNEVKKTRRGTIATLDDSSYSIDGGKTFACGDCRRGQSLIVWAIQEGRKCAASVDKFLMDGTTYLPSNGGIVQRDYKLLKELASQV

SEQ ID NO: 34 - DNA, 1110 bp, Saccharomyces cerevisiae
  source 1..1110: mol_type = other DNA
atggctgaagcaagcatcgaaaagactcaaattttacaaaaatatctagaactggaccaaagaggtagaataattgccgaatacgtttggatcgatggtactggtaacttacgttccaaaggtagaactttgaagaagagaatcacatccattgaccaattgccagaatggaacttcgacggttcttctaccaaccaagcgccaggccacgactctgacatctatttgaaacccgttgcttactacccagatcccttcaggagaggtgacaacattgttgtcttggccgcatgttacaacaatgacggtactccaaacaagttcaaccacagacacgaagctgccaagctatttgctgctcataaggatgaagaaatctggtttggtctagaacaagaatacactctatttgacatgtatgacgatgtttacggatggccaaagggtgggtacccagctccacaaggtccttactactgtggtgttggtgccggtaaggtttatgccagagacatgatcgaagctcactacagagcttgtttgtatgccggattagaaatttctggtattaacgctgaagtcatgccatctcaatgggaattccaagtcggtccatgtaccggtattgacatgggtgaccaattatggatggccagatactttttgcacagagtggcagaagagtttggtatcaagatctcattccatccaaagccattgaagggtgactggaacggtgccggttgtcacactaacgtttccaccaaggaaatgagacaaccaggtggtatgaaatacatcgaacaagccatcgagaagttatccaagagacacgctgaacacattaagttgtacggtagcgataacgacatgagattaactggtagacatgaaaccgcttccatgactgccttttcttctggtgtcgccaacagaggtagctcaattagaatcccaagatccgtcgccaaggaaggttacggttactttgaagaccgtagaccagcttccaacatcgacccatacttggttacaggtatcatgtgtgaaactgtttgcggtgctattgacaatgctgacatgacgaaggaatttgaaagagaatcttca

SEQ ID NO: 35 - protein, 370 aa, Saccharomyces cerevisiae
MAEASIEKTQILQKYLELDQRGRIIAEYVWIDGTGNLRSKGRTLKKRITSIDQLPEWNFDGSSTNQAPGHDSDIYLKPVAYYPDPFRRGDNIVVLAACYNNDGTPNKFNHRHEAAKLFAAHKDEEIWFGLEQEYTLFDMYDDVYGWPKGGYPAPQGPYYCGVGAGKVYARDMIEAHYRACLYAGLEISGINAEVMPSQWEFQVGPCTGIDMGDQLWMARYFLHRVAEEFGIKISFHPKPLKGDWNGAGCHTNVSTKEMRQPGGMKYIEQAIEKLSKRHAEHIKLYGSDNDMRLTGRHETASMTAFSSGVANRGSSIRIPRSVAKEGYGYFEDRRPASNIDPYLVTGIMCETVCGAIDNADMTKEFERESS

SEQ ID NO: 36 - DNA, 5505 bp, Saccharomyces cerevisiae
  source 1..5505: mol_type = other DNA
atgacagttagttccgatacaactgctgaaatatcgttaggttggtcaatccaagactggattgatttccacaagtcatcaagctcccaggcttcactaaggcttcttgaatcactactagactctcaaaatgttgcgccagtcgataatgcgtggatatcgctaatttcaaaggaaaatttactgcaccaattccaaattttaaagagcagagaaaataaagaaactctacctctctacggtgtccctattgctgttaaggacaacatcgacgttagaggtctacccaccaccgctgcatgtccatcctttgcatatgagccttccaaagactctaaagtagtagaactactaagaaatgcaggtgcgataatcgtgggtaagacaaacttggaccaatttgccacaggattagtcggcacacggtctccatatgggaaaacaccttgcgcttttagcaaagagcatgtatctggtggttcctccgctgggtcagcatcggtggtcgccagaggtatcgtaccaattgcattgggtactgatacagcaggttctggtagagtcccagccgccttgaacaacctgattggcctaaagccaacaaagggcgtcttttcctgtcaaggtgtagttcccgcttgtaaatctttagactgcgtctccatctttgcattaaacctaagtgatgctgaacgctgcttccgcatcatgtgccagccagatcctgataatgatgaatattctagaccctatgtttccaaccctttgaaaaaattttcaagcaatgtaacgattgctattcctaaaaatatcccatggtatggtgaaaccaagaatcctgtactgttttccaatgctgtcgaaaatctatcaagaacgggcgctaacgtcatagaaattgattttgagcctcttttagagttagctcgctgtttatacgaaggtacttgggtggccgagcgttatcaagctattcaatcgtttttggacagtaaaccaccaaaggaatctttggaccctactgttatttcaattatagaaggggccaagaaatacagtgcagtagactgcttcagttttgaatacaaaagacaaggcatcttgcaaaaagtgagacgacttctcgaatcagtcgatgtcttgtgtgtgcccacatgtcctttaaatcctactatgcaacaagttgcggatgaaccagtcctagtcaattcaagacaaggcacatggactaattttgtcaacttggcagatttggcagcccttgctgttcccgcagggttccgagacgatggtttgccaaatggtattactttaatcggtaaaaaattcacagattacgcactattagagttggctaaccgctatttccaaaatatattccccaacggttccagaacatacggtacttttacctcttcttcagtaaagccagcaaacgatcaattagtgggaccagactatgacccatctacgtccataaaattggctgttgtcggtgcacatcttaagggtctgcctctacattggcaattggaaaaggtcaatgcaacatatttatgtacaacaaaaacatcaaaagcttaccagctttttgctttgcccaaaaatggaccagttttaaaacctggtttgagaagagttcaagatagcaatggctctcaaatcgaattagaagtgtacagtgttccaaaagaactgttcggtgcttttatttccatggttcctgaaccattaggaataggttcagtggagttagaatctggtgaatggatcaaatcctttatttgtgaagaatctggttacaaagccaaaggtacagttgatatcacaaagtatggtggatttagagcatattttgaaatgttgaagaaaaaagagtcccaaaagaagaagttatttgataccgtgttaattgccaatagaggtgaaattgccgttcgtattatcaagacattaaaaaaattgggtattagatcagttgcagtttattccgaccctgataaatattctcaacacgttactgatgcagatgtttctgtaccccttcatggcacaaccgcagcccaaacttatttagacatgaataagatcatagatgccgctaagcaaactaatgcacaggccattattcctggttatggtttcttgtcggaaaatgcggatttttctgatgcgtgcaccagtgctggcattacctttgttggtccttcgggagatattatcagaggtttagggttaaaacattctgctagacagattgcacagaaggctggcgttcctctagtgccaggctctttgcttatcacatcagttgaagaggctaagaaagtcgcagcggaattggaatacccagttatggtgaagtcaactgctggtggcggtggtattggtttgcagaaagtcgattctgaagaggacatcgagcatatttttgagactgtgaaacatcaaggtgaaacatttttcggtgacgctggtgtatttctggaacggtttatcgaaaatgccaggcatgttgaagtccaacttatgggagatggttttggtaaggccattgctttgggcgaacgtgattgttctttacagcgtcgtaaccaaaaagttatcgaagaaactcctgcaccaaatttgccagaaaagacgaggttggcgttaagaaaggcagctgaaagtttgggatctttattgaattacaagtgtgctggtacggttgaatttatttacgatgagaaaaaggacgagttttactttttagaagttaatacaagattacaagttgaacatccaataacagaaatggttacagggttagacttggtcgagtggatgatcaggattgccgctaatgatgcacctgattttgattctacaaaggtagaagtcaatggggtttcaatggaggcacgtttatatgctgaaaatccattgaaaaatttcagaccttctccaggtttacttgtcgatgtgaaatttcctgattgggcaagagtggatacttgggttaagaaaggtactaatatttctcccgaatatgatccaacattggccaaaattatcgttcatgggaaagaccgtgatgatgcaatttccaagttaaatcaagcgttagaagaaacaaaagtttacggatgtattactaacattgactacctgaagtctatcattaccagtgatttctttgctaaagcaaaagtttctacaaacattttgaactcttatcaatatgagcctaccgccatcgaaattactttgcccggtgcacacactagtattcaggattaccccggtagagttgggtactggagaattggtgttccgccctctggtccaacggacgcatattcgtttagattggcgaacagaattgttggtaatgactacaggactcctgccattgaagtaacgttgactggtccatccatcgttttccattgtgaaactgtcattgccattactggtggtaccgctctatgtacattagacggccaagaaattccccaacacaaaccggtcgaagttaagaggggatctactttatccattggcaagttgacaagcggctgtagagcatacttaggtatcaggggtggcattgatgtgcctaaatacttgggctcttattctactttcactctaggaaatgtcggtggatacaatggaagggtgctaaaacttggagacgtactattcttaccaagcaatgaagaaaataaatcagttgagtgccttccacagaatattcctcaatcattaattcctcaaatttccgaaactaaggaatggagaattggtgtaacatgtggtccccatgggtctccagatttttttaaacctgagtccatcgaagaatttttcagtgagaagtggaaggttcattacaactccaatagatttggtgtccgtttgattggacctaaacctaagtgggcaagaagtaatggtggtgaaggtggtatgcatccttcaaacactcacgattacgtttattctctgggtgcaattaatttcacgggtgatgagccagttattattacttgcgatggtccttccttaggtggttttgtgtgtcaagctgttgtcccagaagcagaactgtggaaggttggacaggttaaacccggtgattccattcagtttgtgccactttcttacgaaagctcgagatccttaaaggaatctcaggatgttgcaattaaatcattggatggtactaagttaaggcgcttagactctgtttcaattttaccatcattcgaaacgcctattcttgcacaaatggaaaaagtgaatgagctttcaccaaaggttgtatacagacaagcaggtgatcgttatgttttggtggaatacggtgataatgaaatgaattttaatatttcctatagaattgaatgcctgatctcccttgtgaaaaagaataagactattggtattgttgaaatgtcccaaggtgttagatctgtattgatagaatttgatggttacaaagtcactcaaaaagaattgcttaaagtattggtggcatatgaaacagaaatccagtttgatgaaaattggaagataacttctaatataataagattaccgatggctttcgaagactcgaagactttggcatgtgttcaaaggtatcaagaaacaattcgttcgtctgctccatggttgccaaataacgttgatttcattgccaatgtaaatggaatttcaaggaatgaagtttatgatatgttgtattctgccagatttatggttttaggtttaggtgatgtcttcctagggtcgccttgtgctgttccattagatcctcgtcacagatttttgggaagcaagtacaacccaagtagaacatatacagaaagaggtgcagtcggtattggcggtatgtatatgtgcatatatgctgctaacagtcctggtgggtaccaattagtgggtagaacaataccaatttgggacaaactatgtctggccgcatcttctgaggttccgtggttgatgaacccatttgaccaagtcgaattttacccagtttctgaagaagatttggataaaatgactgaagattgtgataatggtgtttataaagtcaatatcgaaaagagtgtttttgatcatcaagaatacttgagatggatcaacgcaaacaaagattccatcacagcattccaggagggccagcttggtgaaagagcagaggaatttgccaaattgattcaaaatgcaaactctgaactaaaagaaagtgtcacagtcaaacctgacgaggaagaagacttcccagaaggtgcagaaattgtatattctgagtattctgggcgtttttggaaatccatagcatctgttggagatgttattgaagcaggtcaagggctactaattattgaagccatgaaagcggaaatgattatatccgctcctaaatcgggtaagattatcaagatttgccatggcaatggtgatatggttgattctggtgacatagtggccgtcatagagacattggca

SEQ ID NO: 37 - protein, 1835 aa, Saccharomyces cerevisiae
MTVSSDTTAEISLGWSIQDWIDFHKSSSSQASLRLLESLLDSQNVAPVDNAWISLISKENLLHQFQILKSRENKETLPLYGVPIAVKDNIDVRGLPTTAACPSFAYEPSKDSKVVELLRNAGAIIVGKTNLDQFATGLVGTRSPYGKTPCAFSKEHVSGGSSAGSASVVARGIVPIALGTDTAGSGRVPAALNNLIGLKPTKGVFSCQGVVPACKSLDCVSIFALNLSDAERCFRIMCQPDPDNDEYSRPYVSNPLKKFSSNVTIAIPKNIPWYGETKNPVLFSNAVENLSRTGANVIEIDFEPLLELARCLYEGTWVAERYQAIQSFLDSKPPKESLDPTVISIIEGAKKYSAVDCFSFEYKRQGILQKVRRLLESVDVLCVPTCPLNPTMQQVADEPVLVNSRQGTWTNFVNLADLAALAVPAGFRDDGLPNGITLIGKKFTDYALLELANRYFQNIFPNGSRTYGTFTSSSVKPANDQLVGPDYDPSTSIKLAVVGAHLKGLPLHWQLEKVNATYLCTTKTSKAYQLFALPKNGPVLKPGLRRVQDSNGSQIELEVYSVPKELFGAFISMVPEPLGIGSVELESGEWIKSFICEESGYKAKGTVDITKYGGFRAYFEMLKKKESQKKKLFDTVLIANRGEIAVRIIKTLKKLGIRSVAVYSDPDKYSQHVTDADVSVPLHGTTAAQTYLDMNKIIDAAKQTNAQAIIPGYGFLSENADFSDACTSAGITFVGPSGDIIRGLGLKHSARQIAQKAGVPLVPGSLLITSVEEAKKVAAELEYPVMVKSTAGGGGIGLQKVDSEEDIEHIFETVKHQGETFFGDAGVFLERFIENARHVEVQLMGDGFGKAIALGERDCSLQRRNQKVIEETPAPNLPEKTRLALRKAAESLGSLLNYKCAGTVEFIYDEKKDEFYFLEVNTRLQVEHPITEMVTGLDLVEWMIRIAANDAPDFDSTKVEVNGVSMEARLYAENPLKNFRPSPGLLVDVKFPDWARVDTWVKKGTNISPEYDPTLAKIIVHGKDRDDAISKLNQALEETKVYGCITNIDYLKSIITSDFFAKAKVSTNILNSYQYEPTAIEITLPGAHTSIQDYPGRVGYWRIGVPPSGPMDAYSFRLANRIVGNDYRTPAIEVTLTGPSIVFHCETVIAITGGTALCTLDGQEIPQHKPVEVKRGSTLSIGKLTSGCRAYLGIRGGIDVPKYLGSYSTFTLGNVGGYNGRVLKLGDVLFLPSNEENKSVECLPQNIPQSLIPQISETKEWRIGVTCGPHGSPDFFKPESIEEFFSEKWKVHYNSNRFGVRLIGPKPKWARSNGGEGGMHPSNTHDYVYSLGAINFTGDEPVIITCDGPSLGGFVCQAVVPEAELWKVGQVKPGDSIQFVPLSYESSRSLKESQDVAIKSLDGTKLRRLDSVSILPSFETPILAQMEKVNELSPKVVYRQAGDRYVLVEYGDNEMNFNISYRIECLISLVKKNKTIGIVEMSQGVRSVLIEFDGYKVTQKELLKVLVAYETEIQFDENWKITSNIIRLPMAFEDSKTLACVQRYQETIRSSAPWLPNNVDFIANVNGISRNEVYDMLYSARFMVLGLGDVFLGSPCAVPLDPRHRFLGSKYNPSRTYTERGAVGIGGMYMCIYAANSPGGYQLVGRTIPIWDKLCLAASSEVPWLMNPFDQVEFYPVSEEDLDKMTEDCDNGVYKVNIEKSVFDHQEYLRWINANKDSITAFQEGQLGERAEEFAKLIQNANSELKESVTVKPDEEEDFPEGAEIVYSEYSGRFWKSIASVGDVIEAGQGLLIIEAMKAEMIISAPKSGKIIKICHGNGDMVDSGDIVAVIETLA

SEQ ID NO: 38 - DNA, 2205 bp, Saccharomyces cerevisiae
  source 1..2205: mol_type = other DNA
atgggagaatttaaacctccgctacctcaaggcgctgggtacgctattgtattgggcctaggggccgtatttgcaggaatgatggttttgaccacttatttactgaaacgttatcaaaaggaaatcatcacagcagaagaattcaccaccgccggtagatctgtaaaaaccggcttagtggctgcagccgtggtttctagttggatctggtgttctacattgttaacgtcgtcaacaaaggaatatgcagacggtatatttggcggttatgcgtacgctgctggcgcatgcttccaaattattgcattcgcaattttggcaattaaaaccaagcaaatggctcccaatgcgcacacatatttagaattagtgagaacaagatatggtaagatcggccatggttgctacttgttttatgccatcgcgacgaatattttagtcacttcaatgcttttaacttcaggttctgctgtctttagtgatttaaccgggatgaacactatcgcatcatgttttttactgcctgtgggtgttgttgtttatactctatttggtgggattaaagcaactttcttaacggactatatgcacacatgtgtcattatcatcattgtcctcgtatttgcctttaaagtttatgcgactagtgatgttttaggctcaccgggaaaagtttatgacttagttcgtgaagccgccaagaggcatccagtagacggtaactatcagggtgaatatatgaccatgacatccaaatccgctggtattttattaattattaacctgattggaaatttcggcaccgttttccttgataatggttattggaataaagcgatttctgctagtcccgcagcgagtttgaaagcatatgccatcggtgggttagcatggtttgcagtaccttctttgatttcattgaccatgggattagcatgtcttgcggtggaaacgtctccgaacttccccacctatcctgatccacttacttcgttccaggcaaattctgggttagtcttgccggcagctgcaattgctatcatgggtaaggggggtgctgtggcatcgctgctaatgattttcatggccgtcacatctgctatgtctgctgaactaattgccgtttcatctgttttcacttacgatatctatagagaatatattgatcctcgtgcaagcggtaagaaattgatttacacatcacacgttgcttgtatcttttttggtcttgccatgagtggattttcggttggtttatactatggtggtatttctatgggttatatctatgaaatgatgggtataattattagtagtgcagtattacctgtcgttttgaccttatgttccaaagacatgaatttggtggccgctgtagtgtcgcctattttgggcacaggactggctataatgtcatggcttgtctgtaccaaatccctttataaagaattgaccgtggatactacgttcatggattatccaatgttaacaggtaacttggtggctttgctatcaccagccatttttattcctattttaacgtatgtgtttaagccacaaaattttgactgggagaaaatgaaagatattactagagttgacgaaactgcagagttagttcaggctgaccctgatatccagctttacgatgctgaagctaacgataaggaacaagaagaagaaacaaattctctggtctcagatagtgaaaaaaacgatgttagagtaaataatgaaaaattgattgagcctaaccttggtgttgtaataagtaatgccatttttcaagaagatgacacacagttacaaaatgaattagacgaagaacaaagagaactagcacgtggtttaaaaattgcatacttcctatgtgtttttttcgctttggcatttttggtagtttggcccatgcccatgtatggttccaaatatatcttcagtaaaaaattctttaccggttgggttgttgtgatgatcatctggctttttttcagtgcgtttgccgtttgtatttatccactctgggaaggtaggcatggtatatataccactttgcgaggcctttactgggatctatctggtcaaacttataaattaagggaatggcaaaattcgaacccacaagatctgcatgtagtaacaagccaaattagtgcgagagcacatagacaatcatcacatttcggacaagttgatgaaataatt

SEQ ID NO: 39 - protein, 735 aa, Saccharomyces cerevisiae
MGEFKPPLPQGAGYAIVLGLGAVFAGMMVLTTYLLKRYQKEIITAEEFTTAGRSVKTGLVAAAVVSSWIWCSTLLTSSTKEYADGIFGGYAYAAGACFQIIAFAILAIKTKQMAPNAHTYLELVRTRYGKIGHGCYLFYAIATNILVTSMLLTSGSAVFSDLTGMNTIASCFLLPVGVVVYTLFGGIKATFLTDYMHTCVIIIIVLVFAFKVYATSDVLGSPGKVYDLVREAAKRHPVDGNYQGEYMTMTSKSAGILLIINLIGNFGTVFLDNGYWNKAISASPAASLKAYAIGGLAWFAVPSLISLTMGLACLAVETSPNFPTYPDPLTSFQANSGLVLPAAAIAIMGKGGAVASLLMIFMAVTSAMSAELIAVSSVFTYDIYREYIDPRASGKKLIYTSHVACIFFGLAMSGFSVGLYYGGISMGYIYEMMGIIISSAVLPVVLTLCSKDMNLVAAVVSPILGTGLAIMSWLVCTKSLYKELTVDTTFMDYPMLTGNLVALLSPAIFIPILTYVFKPQNFDWEKMKDITRVDETAELVQADPDIQLYDAEANDKEQEEETNSLVSDSEKNDVRVNNEKLIEPNLGVVISNAIFQEDDTQLQNELDEEQRELARGLKIAYFLCVFFALAFLVVWPMPMYGSKYIFSKKFFTGWVVVMIIWLFFSAFAVCIYPLWEGRHGIYTTLRGLYWDLSGQTYKLREWQNSNPQDLHVVTSQISARAHRQSSHFGQVDEII

SEQ ID NO: 40 - DNA, 1134 bp, Zea mays
  source 1..1134: mol_type = other DNA
atggcgagagcgctgccgctggtgctcgcggcgtccgtcctgctggcggccgccgcgctgctgctggccgcggcgccggcgccggcggccgcaggtcgacttcggcgcggaggacctggcgtcgagaggaggcgctgtgggcgctgtacgagcgctggcgcggccgccacgcggtggcgcgggacctgggcgacaaggcgcgccgtcttcaacgtcttcaaggccaacgtgcgcctcatccacgagttcaaccgccgggacgagccctacaagctccgcctcaaccgcttcggcgacatgaccgccgacgagttccgacgccactacgcgggctccagggtcgcgcaccaccgcatgttccgcggcgacaggcagggctcctcggcgtcggcgtcgttcatgtacgccgacgcgcgcgacgtcccggcctccgtcgactggaggcagaagggcgccgtcaccgacgtcaaggaccagggccagtgcggtagctgctgggcgttctcgacgatcgccgccgtggagggcatcaacgcgatcaagaccaagaacctgacgtcgctgtcggagcagcagctggtggactgcgacaccaaggccaacgccggctgcaacgggggcctcatggactacgcgttccagtacatcgccaagcacggcggggtggcggcggaggacgcgtacccgtacagagcgcgccaggcgtcctgcaagaagtcgccggccccggtcgtcaccatcgacggctacgaggacgtgccggccaacgacgagtcggcgctcaagaaggcggtggcgcaccagcccgtgtccgtcgccatcgaggccagtggctcgcacttccagttctactccgagggggtcttctccggcaggtgcgggacggagctggaccacggcgtcgcggcggtcggctacggggtcaccgcggacggcaccaagtactggctggtcaagaactcgtggggccccgagtggggcgagaaggggtacatccgcatggcgcgcgacgtggccgccaaggagggacactgcggcatcgccatggaggcatcctaccccgtcaagacctcgcccaaccccaaggtccacgccgtcgtcgacgaggacggctccagccacgacgagctctga

SEQ ID NO: 41 - protein, 376 aa, Zea mays
MARALPLVLAASVLLAAALLLLAFAPAPAAAVDFGAEDLASEEALWALYERWRGRHALARDLGDKARRFNVFKANVRLIHEFNRRDEPYKLRLNRFGDMTADEFRRHYAGSRVAHHRMFRGDRQGSSASASFMYADARDVPASVDWRQKGAVTDVKDQGQCGSCWAFSTIAAVEGINAIKTKNLTSLSEQQLVDCDTKANAGCNGGLMDYAFQYIAKHGGVAAEDAYPYRARQASCKKSPAPVVTIDGYEDVPANDESALKKAVAHQPVSVAIEASGSHFQFYSEGVFSGRCGTELDHGVTAVGYGVTADGTKYWLVKNSWGPEWGEKGYIRMARDVAAKEGHCGIAMEASYPVKTSPNPKVHAVVDEDGSSHDEL

SEQ ID NO: 42 - DNA, 1413 bp, Zea mays
  source 1..1413: mol_type = other DNA
atggctgctctgggccgtggcctgcccctcctcctcctgctcctcctcctcgccgtatcgggggccgccaacgccgcggcggcgcccggcggcatgtccatcatcacctacaacgaggagcacggcgcgcgggggctggagcggacggagccggaggtgcgggccatgtacgacctctggctcgcggagcacggccgcgcctacaacgcgctgggcgagggcgagggcgagcgcgaccgccgcttcctcgtcttctgggacaacctccgcttcgtcgacgcgcacaacgagcgcgccggcgcccgcggcttccgcctcgggatgaaccagttcgccgacctcaccaacgacgagttccgcgccgcgtacctcggcgccatggtccccgccgcccgccggggagccgtcgtgggggagaggtaccgccacgacggcgccgccgaggagctgccggagagcgtcgactggagggagaagggcgccgtcgcgcccgtcaagaaccagggkcaatgcggaagttgctgggctttctctgcagtaagctcagtggaaagcgttaaccagatcgtcaccggtgagatggtgacactgtctgaacaggagctcgtagagtgctcgactgacggagggaacagcggctgcaacggcgggctcatggacgccgctttcgatttcatcataaagaacgggggcatcgataccgaagatgactacccttacagagccgtggacgggaagtgcgacatgaacaggaaaaatgccagggttgtgagcatcgatggctttgaagacgtgcctgagaacgacgagaagtcgctgcagaaggcggttgctcaccagccagttagcgttgccattgaggccggaggccgggagttccagctctacaaatcgggtgtcttcagcggaagctgcaccacgaaccttgaccatggtgtcgtcgcggtcggctacggcgccgagaacgggaaggactactggatcgtccgcaactcgtggggcccgaagtggggcgaggctggttacatccgtatggagaggaacgtcaacgccagcactgggaagtgcgggatcgcgatgatggcgtcctacccgacgaagaagggcgcgaaccctcccaggccgtctccaaccccgccaacaccgccggctgcccctgacaatgtctgcgacgagaacttctcgtgctccgcgggcagcacctgctgctgcgcgtttggcttcaggaacgtctgcttggtctggggctgctgcccggtcgagggcgccacctgctgcaaggatcacgccagctgctgcccgccgggctaccctgtctgcaacgtcagagctggaacttgctcggtgagcaagaacagcccactgagcgtcaaggccttgaagcgcactctcgccaagctgagcaccgcatga

SEQ ID NO: 43 - protein, 470 aa, Zea mays
MAALGRGLPLLLLLLLLAVSGAANAAAAPGGMSIITYNEEHGARGLERTEPEVRAMYDLWLAEHGRAYNALGEGEGERDRRFLVFWDNLRFVDAHNERAGARGFRLGMNQFADLTNDEFRAAYLGAMVPAARRGAVVGERYRHDGAAEELPESVDWREKGAVAPVKNQGQCGSCWAFSAVSSVESVNQIVTGEMVTLSEQELVECSTDGGNSGCNGGLMDAAFDFIIKNGGIDTEDDYPYRAVDGKCDMNRKNARVVSIDGFEDVPENDEKSLQKAVAHQPVSVAIEAGGREFQLYKSGVFSGSCTTNLDHGVVAVGYGAENGKDYWIVRNSWGPKWGEAGYIRMERNVNASTGKCGIAMMASYPTKKGANPPRPSPTPPTPPAAPDNVCDENFSCSAGSTCCCAFGFRNVCLVWGCCPVEGATCCKDHASCCPPGYPVCNVRAGTCSVSKNSPLSVKALKRTLAKLSTA

SEQ ID NO: 44 - DNA, 1512 bp, Zea mays
  source 1..1512: mol_type = other DNA
atggctgcctccaccacggcggcggcggcgctgctgctgctcctcctgtcgctcgccgcggcggcggacatgtcgatcgtgtcctacggggagcgcagckwcgaggaggcgcggcggatgtacgcggagtggatggcggcgcacggccggacctacaacgccgtcggcgaggaggagcggcggtaccaggtgttccgggacaacctccgctacatcgacgcgcacaacgccgccgccgacgcgggcgtccactccttccgcctcggcctcaaccgcttcgccgacctcaccaacgacgagtaccgcgccacctacctcggcgccaggaccaggccgcagagggagaggaagctcggcgccaggtaccacgccgccgacaacgaggacctgccggagtccgtcgactggagggccaagggcgccgtcgccgaggtcaaggaccagggcagctgcgggagctgttgggctttctcaacaatagcagctgtggaaggcatcaaccagattgttacaggcgacttgatctccttgtctgagcaagagcttgtcgactgtgacacttcgtacaatcaggggtgcaatggaggtctgatggactatgcgtttgagttcatcatcaacaatggcggaatcgacaccgagaaggattacccttacaaaggcacggacggacgatgtgatgtcaacaggaaaaacgcgaaggttgtcactattgacagctacgaagatgtaccagcgaacgatgagaagagtctgcagaaggcagttgcaaaccagcctgtcagtgtcgcaattgaggctgctggcacagcatttcagctctacagctcgggtatcttcactggaagctgtggaacagcgctagaccatggtgtcacggccgtcggctacggcacagagaacggcaaggactactggatcgtgaagaactcatggggcagcagctggggcgagtccgggtacgtgaggatggagcgcaacatcaaggcgtccagcggcaagtgcggtatcgcggttgagccgtcatacccgttgaaggagggcgctaacccgccgaaccctggccccagcccgccgtccccgaccccggcgcccgccgtctgcgacaactactactcgtgccctgacagcaccacctgctgctgcatctacgagtacggcaagtactgcttcgcctggggctgctgcccgctcgagggcgccacctgctgcgacgatcactacagctgctgcccccatgactaccccatctgcaacgtcaggcagggaacctgcctcatgggcaaggacagcccactgtcactgtcagtgaaggctacgaagcgaaccctggccaagccgcactgggctttctccggcaacacagctgacggcatgaagagcagcgcatgagaaaacgtgggcaaggacagcccactgtcactgtcagtgaaggctacgaagcgaaccctggccaagccgcactgggctttctccggcaacacagctgacggcatgaagagcagcgcatga

SEQ ID NO: 45 - protein, 463 aa, Zea mays
  SITE 34: note = misc_feature - Xaa can be any naturally occurring amino acid
MAASTTAAAALLLLLLSLAAAADMSIVSYGERSXEEARRMYAEWMAAHGRTYNAVGEEERRYQVFRDNLRYIDAHNAAADAGVHSFRLGLNRFADLTNDEYRATYLGARTRPQRERKLGARYHAADNEDLPESVDWRAKGAVAEVKDQGSCGSCWAFSTIAAVEGINQIVTGDLISLSEQELVDCDTSYNQGCNGGLMDYAFEFIINNGGIDTEKDYPYKGTDGRCDVNRKNAKVVTIDSYEDVPANDEKSLQKAVANQPVSVAIEAAGTAFQLYSSGIFTGSCGTALDHGVTAVGYGTENGKDYWIVKNSWGSSWGESGYVRMERNIKASSGKCGIAVEPSYPLKEGANPPNPGPSPPSPTPAPAVCDNYYSCPDSTTCCCIYEYGKYCFAWGCCPLEGATCCDDHYSCCPHDYPICNVRQGTCLMGKDSPLSLSVKATKRTLAKPHWAFSGNTADGMKSSA

SEQ ID NO: 46 - DNA, 1359 bp, Neurospora crassa
  source 1..1359: mol_type = other DNA
atggatcttgacggcatcccttctctcaacaacggaagccttcgccattcgacaaaagcaagatgcagataccccaaagatggtggatactatgtcaatgtgaccattggtacacccggaaggaatctttccctacatctcgacacaggctcaagtgatacttgggtgaattctcccagttcgattctttgtcaagatgaagacaaaccatgcgaatactccggcacctacctagccaatgactcgtctacttatgagtacatcagcaatcatttcgacatcaagtatgtggatgggtctggcgccagaggggactatgcctccgatactttcacaatcggcaacaccaaactgaaccggctgcagtttggcattggctactcttcaactaacgcgcagggactgctcggtatagggtacaccctgagcgaagtgcaaacccgtgccgggttgccagcctataacaaccttccggcgcaaatggtcgccgacggcttgatcaactccaacgcctactccatctggctcaatgacctcgatgccctcacgggtaccattctcttcggcggcgtcgacgccgccaagtacgagggcgacctgctcaccctacccgttcaaaccccagaaaagggcacgtacaagaacctcatggtcaccatgaccggcctgtctctatctcagtcccagtcctcctccagcgacaagggcaacggtgatgacaccacccagatctccaaagacaacctcgcccttgccgttctcttggacaccggctcaaccctcagttatctccccagtgagctcgtcaaaccgctctacgacgcgatcggtatcgaatacataacagaccccgacggcaaagtagatggctacgcgccctgccatctcatgtcctcgtcgcaatccgtcatgttctccttctcctccccgcttcagattgccgtgcccatgaacgagctcatcgtcaaccggaccttccacggaaaactcccacgcatgccggacggcgtcacagacgcctgcatctttggcatccaagaacgtaatggcacgggagcaaacaccttgggcgacacgttcctgcgcagcgcgtatgttgtgtttgatttggacaacaacgagatctcgatggcgcagacgaggtttaatgccacggcgacggacctgaaggagattaagaagggcaaaggtggggtgccgggcgcgaaggcggtggaaaatcctgtggaggccacgagcggcctgtctggtaatgaaggggggatatatgtgaatggggcggcgtgtgaattgaatgtgggtatgggaatggcttggggtttgttggttggtgctacgatggtggttttggggttgtga

SEQ ID NO: 47 - protein, 452 aa, Neurospora crassa
MDLDGIPSLNNGSLRHSTKARCRYPKDGGYYVNVTIGTPGRNLSLHLDTGSSDTWVNSPSSILCQDEDKPCEYSGTYLANDSSTYEYISNHFDIKYVDGSGARGDYASDTFTIGNTKLNRLQFGIGYSSTNAQGLLGIGYTLSEVQTRAGLPAYNNLPAQMVADGLINSNAYSIWLNDLDALTGTILFGGVDAAKYEGDLLTLPVQTPEKGTYKNLMVTMTGLSLSQSQSSSSDKGNGDDTTQISKDNLALAVLLDTGSTLSYLPSELVKPLYDAIGIEYITDPDGKVDGYAPCHLMSSSQSVMFSFSSPLQIAVPMNELIVNRTFHGKLPRMPDGVTDACIFGIQERNGTGANTLGDTFLRSAYVVFDLDNNEISMAQTRFNATATDLKEIKKGKGGVPGAKAVENPVEATSGLSGNEGGIYVNGAACELNVGMGMAWGLLVGATMVVLGL

SEQ ID NO: 48 - DNA, 1443 bp, Podospora anserina
  source 1..1443: mol_type = other DNA
atgaagacagccacttctcttttggttgttgcagcctcgcttgcgggtcagacgatagacgctctctctttgccatcgacgacgccaacacaacaacaacaacgaagagatggaagtggaccgagggttgtggggatggatattcagaggaggacgcccaaaaacccattacatcgagatcatctaagaaagagagggagcgtggaggtcgatttggacaatcaggaaaccctctacttcatcaacggcacaatcggcaccccaccaaaatccctccgtctacacctcgacaccggctcgtccgacctctgggtcaacaccccatcctcctccctatgcacccagtcctcagcgccctgtaaatacgccggcacttactcggccaacggctcaagcacgtacgagtacatcggctcctggttcaacatctcctacgtcgacggctccggcgcctcgggggattacgtttccgacaccgtcacctttggcgacgccacgctggacaggttgcagtttggcataggatactcgtccaacaacgcccagggcatcctcgggatagggtacccgatcaacgaagtccaagtcggcagggcaggaatgagaccttacaacaacctccccgcgcaaatggtggccgacgggctgatccaaacaaacgcttattccctgtggctgaacgacctcgacgctgacacggggaatattctgtttggcggtgtcgacacggaaaagtttgttcccccgttgatgtccctcccggtggaatccgaggcgggggtgtacgccgagtttatgatcacgttgacgaaagtcgagctcggctcggcgcaggtaggcggcgatctcgccttggctgtgctgttggataccggcagcagcttgacgtacctccccgaccggatggtgcaagatattttcgaccttgtcgacgcgcagtacgatcctgaggctaacgcggcgtatgttccttgctcgcttgccgacaatgagactgctgttttgtcgtttacgtttacggagccgacgatcaatgtggggatggatgagcttgtgctcgaccttgtgacgagctcggggaggaggccggtttttagtgacgggacggaggcgtgcctgtttgggattgcgccggcgggggaggggacgaatgtgctgggggatacgtttttgaggagtgcgtatgtggtttatgacttggagaataacgagatttcgttggcggcgacgaggttcaacagtacggggacgagggttgaggagatagggaagggggaggggggggtgccgggggcgacgaaggtggagaatccgaccaaggcgacggaggggttggatgggccgaatggtttgggggggataagtgctgggaataagaggggtttggaggttggggtggtgtggttggtggcgggtatggtgggagttttgctggtggtctaa

SEQ ID NO: 49 - protein, 480 aa, Podospora anserina
MKTATSLLVVAASLAGQTIDALSLPSTTPTQQQQRRDGSGPRVVGMDIQRRTPKNPLHRDHLRKRGSVEVDLDNQETLYFINGTIGTPPKSLRLHLDTGSSDLWVNTPSSSLCTQSSAPCKYAGTYSANGSSTYEYIGSWFNISYVDGSGASGDYVSDTVTFGDATLDRLQFGIGYSSNNAQGILGIGYPINEVQVGRAGMRPYNNLPAQMVADGLIQTNAYSLWLNDLDADTGNILFGGVDTEKFVPPLMSLPVESEAGVYAEFMITLTKVELGSAQVGGDLALAVLLDTGSSLTYLPDRMVQDIFDLVDAQYDPEANAAYVPCSLADNETAVLSFTFTEPTINVGMDELVLDLVTSSGRRPVFSDGTEACLFGIAPAGEGTNVLGDTFLRSAYVVYDLENNEISLAATRFNSTGTRVEEIGKGEGGVPGATKVENPTKATEGLDGPNGLGGISAGNKRGLEVGVVWLVAGMVGVLLVV

SEQ ID NO: 50 - DNA, 1425 bp, Magnaporthe oryzae
  source 1..1425: mol_type = other DNA
atgaggttaatagcatcactggccttggcggcatcactggcctcttcaatcacaaatggatcaacgataccgaggagggcctcgggcacaccagctgcgccgagggtaattggcctggagacggaaaggcagcacatcccgaatcccttggagcgggacaggttaaggaggagagccgccgtcatggctactctcgacaacgagcagacgctgtactttgtcaacgtctccatcggcacgccaccgcagaagctacgtcttcaccttgacaccggcagcagtgacctgtgggtcaacacgcccgactcgaaactctgttccgtctcgtcgcagccgtgcagatttgccggaacctttagcgcaaacagctcctctacatatcaatacatcaacagtgtcttcaacatctcgtacgttgacggctcaggcgccaatggcgactatgtctccgatatggtcacggtgggcaacaccaagatcgaccgcctccagttcggaatcggatacacgagcagttcggcgcagggaattctgggcgtcggttatgaggccaacgaggtgcaggtcggacgtgctcagctcaagccgtaccgcaacctcccctcgcgcatggtcgaggagggcctgatcgcttccaacgcctacagcctttacctcaatgacctgcagtccaacaagggcagcatcctgtttggtggcatcgacacggagcagtacacgggaacgctgcagaccgtcccgatccagcccaacggtggccgcatggccgagtttctcatcaccttgacctctgtctccttgacctcggcaagcattggcggtgacaagctggccctggccgtgctgctcgactcgggttcctcgctcacctacctgcccgacgacatcgtaaagaacatgtacagcgccgtgggcgcccagtacgacagcaacgagggcgccgcatacgtcccctgctccctcgccagggaccaggccaactcgttgacctttagcttttccggcatccccatcgtcgtgcccatgaacgagctcgtcttggacctggtaacttccaacggccgccggccctcgttccgcaacggcgtccccgcctgcctgtttggcgtcgccccggccggcaagggcaccaatgttctaggtgacacgttcctccgctccgcctacgtcgtctacgacctcgaaaacaacgccatctcgctcgctcagaccagcttcaacgctaccaagagcaacgtcaaggagatcggcaagggcagcaaccctgttcccggcgccgttgccgtctcgcagcccgtcgctgccacttcgggcctgtcgcagaacggcggcaacaggtctggctctggcgccatcgcccgggccgttccgactctcttgctcgtcggaggaatcttctccggttcgttgttgactctgttctag

SEQ ID NO: 51 - protein, 474 aa, Magnaporthe oryzae
MRLIASLALAASLASSITNGSTIPRRASGTPAAPRVIGLETERQHIPNPLERDRLRRRAAVMATLDNEQTLYFVNVSIGTPPQKLRLHLDTGSSDLWVNTPDSKLCSVSSQPCRFAGTFSANSSSTYQYINSVFNISYVDGSGANGDYVSDMVTVGNTKIDRLQFGIGYTSSSAQGILGVGYEANEVQVGRAQLKPYRNLPSRMVEEGLIASNAYSLYLNDLQSNKGSILFGGIDTEQYTGTLQTVPIQPNGGRMAEFLITLTSVSLTSASIGGDKLALAVLLDSGSSLTYLPDDIVKNMYSAVGAQYDSNEGAAYVPCSLARDQANSLTFSFSGIPIVVPMNELVLDLVTSNGRRPSFRNGVPACLFGVAPAGKGTNVLGDTFLRSAYVVYDLENNAISLAQTSFNATKSNVKEIGKGSNPVPGAVAVSQPVAATSGLSQNGGNRSGSGAIARAVPTLLLVGGIFSGSLLTLF

SEQ ID NO: 52 - DNA, 1806 bp, Saccharomyces cerevisiae
  source 1..1806: mol_type = other DNA
atgagtaatacttcttcgtacgagaagaataatccagataatctgaaacacaatggtattaccatagattctgagtttctaactcaggagccaataaccattccctcaaatggctccgctgtttctattgacgaaacaggttcagggtccaaatggcaagactttaaagattctttcaaaagggtaaaacctattgaagttgatcctaatctttcagaagctgaaaaagtggctatcatcactgcccaaactccattgaagcaccacttgaagaatagacatttgcaaatgattgccatcggtggtgccatcggtactggtctgctggttgggtcaggtactgcactaagaacaggtggtcccgcttcgctactgattggatgggggtctacaggtaccatgatttacgctatggttatggctctgggtgagttggctgttatcttccctatttcgggtgggttcaccacgtacgctaccagatttattgatgagtcctttggttacgctaataatttcaattatatgttacaatggttggttgtgctaccattggaaattgtctctgcatctattactgtaaatttctggggtacagatccaaagtatagagatgggtttgttgcgttgttttggcttgcaattgttatcatcaatatgtttggtgtcaaaggttatggtgaagcagaattcgtcttttcatttatcaaggtcatcactgttgttgggttcatcatcttaggtatcattctaaactgtggtggtggtccaacaggtggttacattgggggcaagtactggcatgatcctggtgcctttgctggtgacactccaggtgctaaattcaaaggtgtttgttctgtcttcgtcaccgctgccttttcttttgccggttcagaattggttggtcttgctgccagtgaatccgtagagcctagaaagtccgttcctaaggctgctaaacaagttttctggagaatcaccctattttatattctgtcgctattaatgattggtcttttagtcccatacaacgataaaagtttgattggtgcctcctctgtggatgctgctgcttcacccttcgtcattgccattaagactcacggtatcaagggtttgccaagtgttgtcaacgtcgttatcttgattgccgtgttatctgtcggtaactctgccatttatgcatgttccagaacaatggttgccctagctgaacagagatttctgccagaaatcttttcctacgttgaccgtaagggtagaccattggtgggaattgctgtcacatctgcattcggtcttattgcgtttgttgccgcctccaaaaaggaaggtgaagttttcaactggttactagccttgtctgggttgtcatctctattcacatggggtggtatctgtatttgtcacattcgtttcagaaaggcattggccgcccaaggaagaggcttggatgaattgtctttcaagtctcctaccggtgtttggggttcctactgggggttatttatggttattattatgttcattgcccaattctacgttgctgtattccccgtgggagattctccaagtgcggaaggtttcttcgaagcttatctatccttcccacttgttatggttatgtacatcggacacaagatctataagaggaattggaagcttttcatcccagcagaaaagatggacattgatacgggtagaagagaagtcgatttagatttgttgaaacaagaaattgcagaagaaaaggcaattatggccacaaagccaagatggtatagaatctggaatttctggtgt

SEQ ID NO: 53 - protein, 602 aa, Saccharomyces cerevisiae
MSNTSSYEKNNPDNLKHNGITIDSEFLTQEPITIPSNGSAVSIDETGSGSKWQDFKDSFKRVKPIEVDPNLSEAEKVAIITAQTPLKHHLKNRHLQMIAIGGAIGTGLLVGSGTALRTGGPASLLIGWGSTGTMIYAMVMALGELAVIFPISGGFTTYATRFIDESFGYANNFNYMLQWLVVLPLEIVSASITVNFWGTDPKYRDGFVALFWLAIVIINMFGVKGYGEAEFVFSFIKVITVVGFIILGIILNCGGGPTGGYIGGKYWHDPGAFAGDTPGAKFKGVCSVFVTAAFSFAGSELVGLAASESVEPRKSVPKAAKQVFWRITLFYILSLLMIGLLVPYNDKSLIGASSVDAAASPFVIAIKTHGIKGLPSVVNVVILIAVLSVGNSAIYACSRTMVALAEQRFLPEIFSYVDRKGRPLVGIAVTSAFGLIAFVAASKKEGEVFNWLLALSGLSSLFTWGGICICHIRFRKALAAQGRGLDELSFKSPTGVWGSYWGLFMVIIMFIAQFYVAVFPVGDSPSAEGFFEAYLSFPLVMVMYIGHKIYKRNWKLFIPAEKMDIDTGRREVDLDLLKQEIAEEKAIMATKPRWYRIWNFWC

SEQ ID NO: 54 - DNA, 1062 bp, Saccharomyces cerevisiae
  source 1..1062: mol_type = other DNA
atgatgaataacaacggcaaccaagtgtcgaatctctccaatgcgctccgtcaagtaaacataggaaacaggaacagtaatacaaccaccgatcaaagtaatataaattttgaattttcaacaggtgtaaataataataataataacaatagcagtagtaataacaataatgttcaaaacaataacagcggccgcaatggtagccaaaataatgataacgagaataatatcaagaataccttagaacaacatcgacaacaacaacaggcattttcggatatgagtcacgtggagtattccagaattacaaaattttttcaagaacaaccactggagggatatacccttttctctcacaggtctgcgcctaatggattcaaagttgctatagtactaagtgaacttggatttcattataacacaatcttcctagatttcaatcttggcgaacatagggcccccgaatttgtgtctgtgaaccctaatgcaagagttccagctttaatcgatcatggtatggacaacttgtctatttgggaatcaggggcgattttattacatttggtaaataaatattacaaagagactggtaatccattactctggtccgatgatttagctgaccaatcacaaatcaacgcatggttgttcttccaaacgtcagggcatgcgccaatgattggacaagctttacatttcagatacttccattcacaaaagatagcaagtgctgtagaaagatatacggatgaggttagaagagtttacggtgtagtggagatggccttggctgaacgtagagaagcgctggtgatggaattagacacggaaaatgcggctgcatactcagctggtacaacaccaatgtcacaaagtcgtttctttgattatcccgtatggcttgtaggagataaattaactatagcagatttggcctttgtcccatggaataatgtcgtggatagaattggcattaatatcaaaattgaatttccagaagtttacaaatggacgaagcatatgatgagaagacccgcggtcatcaaggcattgcgtggtgaa

SEQ ID NO: 55 - protein, 354 aa, Saccharomyces cerevisiae
MMNNNGNQVSNLSNALRQVNIGNRNSNTTTDQSNINFEFSTGVNNNNNNNSSSNNNNVQNNNSGRNGSQNNDNENNIKNTLEQHRQQQQAFSDMSHVEYSRITKFFQEQPLEGYTLFSHRSAPNGFKVAIVLSELGFHYNTIFLDFNLGEHRAPEFVSVNPNARVPALIDHGMDNLSIWESGAILLHLVNKYYKETGNPLLWSDDLADQSQINAWLFFQTSGHAPMIGQALHFRYFHSQKIASAVERYTDEVRRVYGVVEMALAERREALVMELDTENAAAYSAGTTPMSQSRFFDYPVWLVGDKLTIADLAFVPWNNVVDRIGINIKIEFPEVYKWTKHMMRRPAVIKALRGE

SEQ ID NO: 56 - DNA, 282 bp, Saccharomyces cerevisiae
  source 1..282: mol_type = other DNA
atggaccgatctttgcaagtatatatctgtatgtatccatatttagatggcagcaagcaatatagatttgatgagcttatatcattttatcgtccttgtccaaaaagtcttgataacattaaaagtcactaccgtcaaatccatcatcaaatccgccgtcgaacccaccagcatcatcaaatccgccgtcggacccaccagcatcatcaccgtagtaattgttctcgacaacgacagtgtctggtccgtcatagttgtggtcgtcaaatgcgtgttctagca

SEQ ID NO: 57 - protein, 94 aa, Saccharomyces cerevisiae
MDRSLQVYICMYPYLDGSKQYRFDELISFYRPCPKSLDNIKSHYRQIHHQIRRRTHQHHQIRRRTHQHHHRSNCSRQRQCLVRHSCGRQMRVLA

SEQ ID NO: 58 - DNA, 499 bp, Saccharomyces cerevisiae
  source 1..499: mol_type = other DNA
gggcgccataaccaaggtatctatagaccgccaatcagcaaactacctccgtacattcctgttgcacccacacatttatacacccagaccgcgacaaattacccataaggttgtttgtgacggcgtcgtacaagagaacgtgggaactttttaggctcaccaaaaaagaaaggaaaaatacgagttgctgacagaagcctcaagaaaaaaaaaattcttcttcgactatgctggagccagagatgatcgagccggtagttaactatatatagctaaattggttccatcaccttcttttctggtgtcgctccttctagtgctatttctggcttttcctatttttttttttccatttttctttctctctttctaatatataaattctcttgcattttctatttttctctctatctattctacttgtttattcccttcaaggttttttttaaggactacttgtttttagaatatacggtcaacgaactataattaactaaac

SEQ ID NO: 59 - DNA, 449 bp, Saccharomyces cerevisiae
  source 1..449: mol_type = other DNA
ccagaaaggcaacgcaaaattttttttccagggaataaactttctatgacccactacttctcgtaggaacaatttcgggcccctgcgtgttcttctgaggttcatcttttacatttgcttctgctggataattttcagaggcaacaaggaaaaattagatggcaaaaagtcgtctttcaaggaaaaatccccaccatccttcgagatcccctgtaacttattggcaactgaaagaatgaaaaggaggaaaatacaaaatatactagaactgaaaaaaaaaagtataaatagagacgatatatgccaatacttcacaatgttcgaatccattcttcatttgcagctattgtaaaataataaaacatcaagaacaaacaagctcaacttgtcttttctaagaacaaagaataaacacaaaaacaaaaagtttttttaattttaatcaaaaa

SEQ ID NO: 60 - DNA, 749 bp, Saccharomyces cerevisiae
  source 1..749: mol_type = other DNA
cgatttttttctaaaccgtggaatatttcggatatccttttgttgtttccgggtgtacaatatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactatctcatataca

SEQ ID NO: 61 - DNA, 450 bp, Saccharomyces cerevisiae
  source 1..450: mol_type = other DNA
ctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaattaaagcaatcacacaattctctcggatgccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaag

SEQ ID NO: 62 - DNA, 500 bp, Saccharomyces cerevisiae
  misc_feature 368: note = n is a, c, g, or t
  misc_feature 452..453: note = n is a, c, g, or t
  source 1..500: mol_type = other DNA
gttaattcaaattaattgatatagttttttaatgagtattgaatctgtttagaaataatggaatattatttttatttatttatttatattattggtcggctcttttcttctgaaggtcaatgacaaaatgatatgaaggaaataatgatttctaaaattatacaacgtaagatatttttacaagagcctagctcatcttttgtcatgcactattttactcacgcttgaaattaacggccagttcactgcggagtcatttcaaagtcatcctaatcgatctatcgtttttgatagctcattttggagttcgcgattgtcttctgttattcacaactgttttaatttttatttcattctggaactcttcnagttctttgtaaagtctttcatagtagcttactttatcctccaacatatttaacttcatgtcaatttcggctcttaaattttcnncatcatcaagttcaacatcatcttttaacttgaatttattctctagc

SEQ ID NO: 63 - DNA, 491 bp, Saccharomyces cerevisiae
  source 1..491: mol_type = other DNA
gcgatttaatctctaattattagttaaagttttataagcatttttatgtaacgaaaaataaattggttcatattattactgcactgtcacttaccatggaaagaccagacaagaagttgccgacagtctgttgaattggcttaagtctgggtccgctcctttctaaatttgaagaatttctcttaaacgatatgtatattcttttcgttggaaaagatgtcttccaaaaaaaaaaaaccgatgaattagtggaaccaaggaaaaaaaagaggtatccttgattaaggaacactgtttaaacagtgtggtttccaaaaacctgaaactgcattagcgtaatagaagactagacacctcgatacaaataatggttactcaattcaaaactgccagcgaattcgactctgcaattgctcaagacaagctagttgtcgtagatttctacgccacttggtgcggtccatgtaaaatgattgctccaatgattgaaa

SEQ ID NO: 64 - DNA, 504 bp, Saccharomyces cerevisiae
  source 1..504: mol_type = other DNA
tcctgttgaagtagcatttaatcataatttttgtcacattttaatcaacttgatttttctggtttaatttttctaattttaattttaatttttttatcaatgggaactgatacactaaaaagaattaggagccaacaagaataagccgcttatttcctactagagtttacttaaaatttcatctcgaattgtcattctaatattttatccacacacacacacaccttaaaatttttagattaaatggcatcaactcttagcttcacacacacacacacaccgaagctggttgttttatttgatttgatataattggtttctctggatggtactttttctttcttggttatttcctattttaaaatatgaaacgcacacaagtcataattattctaatagagcacaattcacaacacgcacatttcaactttaatatttttttagaaacactttatttagtctaattcttaatttttaatatatataatgcacacacactaattt

SEQ ID NO: 65 - DNA, 500 bp, Saccharomyces cerevisiae
  source 1..500: mol_type = other DNA
tagcgtgttacgcacccaaactttttatgaaagtctttgtttataatgatgaggtttataaatatatagtggagcaaagattaatcactaaatcaagaagcagtaccagtattttttttatatcaagtagtgataatggaaatagcccaaatttggcttccgtcggcacatagcacgtttgagagacattatcaccatcaagcatcgagccgcccaaacctaactgtataagttttttcacgtttttgatttttccttgcacacttcgatattactctcacgataaaagggccgaagagaatatttttcttgaacatccagaattttaattcggagaaatttcacaagccgccgatttaagggtcctgtgttcttaataatcagcctctctcaaagcaggtaagaggcagtctttcttttaacaataggagacattcgaactaaaacatcagccccaaaaatgcgcttgaaggtcattaggatttggatttcttcctcat

SEQ ID NO: 66 - DNA, 23 bp, synthetic construct
  misc_feature 1..23: note = X14961
  source 1..23: mol_type = other DNA
gcagttaccttttagcacccaac

SEQ ID NO: 67 - DNA, 24 bp, synthetic construct
  misc_feature 1..24: note = X14966
  source 1..24: mol_type = other DNA
ggtgtaggtaagcagaatgaggag

SEQ ID NO: 68 - DNA, 59 bp, synthetic construct
  misc_feature 1..59: note = X15464
  source 1..59: mol_type = other DNA
gtccatgtaaaatgattgctccaatgattgaaagaggtttagacattggctcttcattg

SEQ ID NO: 69 - DNA, 59 bp, synthetic construct
  misc_feature 1..59: note = X15465
  source 1..59: mol_type = other DNA
ctaagctcaatgaagagccaatgtctaaacctctttcaatcattggagcaatcatttta

SEQ ID NO: 70 - DNA, 60 bp, synthetic construct
  misc_feature 1..60: note = X18846
  source 1..60: mol_type = other DNA
gtccatgtaaaatgattgctccaatgattgaaaagcacgcagcacgctgtatttacgtat

SEQ ID NO: 71 - DNA, 59 bp, synthetic construct
  misc_feature 1..59: note = X18847
  source 1..59: mol_type = other DNA
aattaaatacgtaaatacagcgtgctgcgtgcttttcaatcattggagcaatcatttta

SEQ ID NO: 72 - DNA, 60 bp, synthetic construct
  misc_feature 1..60: note = X18858
  source 1..60: mol_type = other DNA
agccagcttaaagagttaaaaatttcatagctactacttattcccttcgagattatatct

SEQ ID NO: 73 - DNA, 59 bp, synthetic construct
  misc_feature 1..59: note = X18859
  source 1..59: mol_type = other DNA
gttcctagatataatctcgaagggaataagtagtagctatgaaatttttaactctttaa

SEQ ID NO: 74 - DNA, 60 bp, synthetic construct
  misc_feature 1..60: note = X18860
  source 1..60: mol_type = other DNA
acatcatcttttaacttgaatttattctctagcagcacgcagcacgctgtatttacgtat

SEQ ID NO: 75 - DNA, 59 bp, synthetic construct
  misc_feature 1..59: note = X18861
  source 1..59: mol_type = other DNA
aattaaatacgtaaatacagcgtgctgcgtgctgctagagaataaattcaagttaaaag

SEQ ID NO: 76 - DNA, 30 bp, synthetic construct
  misc_feature 1..30: note = X18869
  source 1..30: mol_type = other DNA
agatcctgtggtagtgctgtctgaacagaa

SEQ ID NO: 77 - DNA, 59 bp, synthetic construct
  misc_feature 1..59: note = X18955
  source 1..59: mol_type = other DNA
ataaaattaaatacgtaaatacagcgtgctgcgtgctcgatttttttctaaaccgtgga

SEQ ID NO: 78 - DNA, 60 bp, synthetic construct
  misc_feature 1..60: note = X19513
  source 1..60: mol_type = other DNA
acttggtgcggtccatgtaaaatgattgctccaatgattgaaaatgaggaagaaatccaa

SEQ ID NO: 79 - DNA, 60 bp, synthetic construct
  misc_feature 1..60: note = X19514
  source 1..60: mol_type = other DNA
tgaaggtcattaggatttggatttcttcctcattttcaatcattggagcaatcattttac

SEQ ID NO: 80 - DNA, 59 bp, synthetic construct
  misc_feature 1..59: note = X19551
  source 1..59: mol_type = other DNA
agccagcttaaagagttaaaaatttcatagctagggcgccataaccaaggtatctatag

SEQ ID NO: 81 - DNA, 60 bp, synthetic construct
  misc_feature 1..60: note = X19552
  source 1..60: mol_type = other DNA
tggcggtctatagataccttggttatggcgccctagctatgaaatttttaactctttaag

SEQ ID NO: 82 - DNA, 59 bp, synthetic construct
  misc_feature 1..59: note = X19721
  source 1..59: mol_type = other DNA
aaagaaatgtcagagccagaatttcaacaagctaagctttctaactgatctatccaaaa

SEQ ID NO: 83 - DNA, 60 bp, synthetic construct
  misc_feature 1..60: note = X19722
  source 1..60: mol_type = other DNA
ttttcagttttggatagatcagttagaaagcttagcttgttgaaattctggctctgacat

SEQ ID NO: 84 - DNA, 60 bp, synthetic construct
  misc_feature 1..60: note = X19726
  source 1..60: mol_type = other DNA
atccgaaatattccacggtttagaaaaaaatcggatgctatgtttgaccaaggtgatgta

SEQ ID NO: 85 - DNA, 60 bp, synthetic construct
  misc_feature 1..60: note = X19727
  source 1..60: mol_type = other DNA
ttaaaatacatcaccttggtcaaacatagcatccgatttttttctaaaccgtggaatatt

SEQ ID NO: 86 - DNA, 60 bp, synthetic construct
  misc_feature 1..60: note = X19948
  source 1..60: mol_type = other DNA
aaagaaatgtcagagccagaatttcaacaagctgatgctatgtttgaccaaggtgatgta

SEQ ID NO: 87 - DNA, 60 bp, synthetic construct
  misc_feature 1..60: note = X19949
  source 1..60: mol_type = other DNA
ttaaaatacatcaccttggtcaaacatagcatcagcttgttgaaattctggctctgacat

SEQ ID NO: 88 - DNA, 60 bp, synthetic construct
  misc_feature 1..60: note = X19950
  source 1..60: mol_type = other DNA
atccgaaatattccacggtttagaaaaaaatcgagcacgcagcacgctgtatttacgtat

SEQ ID NO: 89 - DNA, 60 bp, synthetic construct
  misc_feature 1..60: note = X19967
  source 1..60: mol_type = other DNA
tgaaggtcattaggatttggatttcttcctcataaattagtgtgtgtgcattatatatat

SEQ ID NO: 90 - DNA, 60 bp, synthetic construct
  misc_feature 1..60: note = X19968
  source 1..60: mol_type = other DNA
tttttaatatatataatgcacacacactaatttatgaggaagaaatccaaatcctaatga

SEQ ID NO: 91 - DNA, 59 bp, synthetic construct
  misc_feature 1..59: note = X19969
  source 1..59: mol_type = other DNA
aattaaatacgtaaatacagcgtgctgcgtgctccagaaaggcaacgcaaaattttttt

SEQ ID NO: 92 - DNA, 60 bp, synthetic construct
  misc_feature 1..60: note = X19970
  source 1..60: mol_type = other DNA
ccctggaaaaaaaattttgcgttgcctttctggagcacgcagcacgctgtatttacgtat

SEQ ID NO: 93 - DNA, 32 bp, synthetic construct
  misc_feature 1..32: note = X20022
  source 1..32: mol_type = other DNA
aggtagacgctacagtcacaggtgtcacaact

SEQ ID NO: 94 - DNA, 60 bp, synthetic construct
  misc_feature 1..60: note = X20023
  source 1..60: mol_type = other DNA
ggacgaggcaagctaaacagatctctagacctattggtgtacaacttaatttgcagctta

SEQ ID NO: 95 - DNA, 60 bp, synthetic construct
  misc_feature 1..60: note = X20024
  source 1..60: mol_type = other DNA
ccgtttcttttctttggactatcatgtagtctcaggctgctttaaaaacaagaaagaaag

SEQ ID NO: 96 - DNA, 32 bp, synthetic construct
  misc_feature 1..32: note = X20025
  source 1..32: mol_type = other DNA
gagtgggatgcgcatatagtgcatgaacctat

SEQ ID NO: 97 - DNA, 60 bp, synthetic construct
  misc_feature 1..60: note = X20026
  source 1..60: mol_type = other DNA
ttgttttaagctgcaaattaagttgtacaccaaaggctgctttaaaaacaagaaagaaag

SEQ ID NO: 98 - DNA, 60 bp, synthetic construct
  misc_feature 1..60: note = X20027
  source 1..60: mol_type = other DNA
cttcttctttctttcttgtttttaaagcagcctttggtgtacaacttaatttgcagctta

SEQ ID NO: 99 - DNA, 60 bp, synthetic construct
  misc_feature 1..60: note = X20028
  source 1..60: mol_type = other DNA
ttgttttaagctgcaaattaagttgtacaccaataggtctagagatctgtttagcttgcc

SEQ ID NO: 100 - DNA, 60 bp, synthetic construct
  misc_feature 1..60: note = X20029
  source 1..60: mol_type = other DNA
cttcttctttctttcttgtttttaaagcagcctgagactacatgatagtccaaagaaaag

SEQ ID NO: 101 - DNA, 59 bp, synthetic construct
  misc_feature 1..59: note = X20043
  source 1..59: mol_type = other DNA
ataaaattaaatacgtaaatacagcgtgctgcgtgctatgaggaagaaatccaaatcct

SEQ ID NO: 102 - DNA, 59 bp, synthetic construct
  misc_feature 1..59: note = X20044
  source 1..59: mol_type = other DNA
tgaaggtcattaggatttggatttcttcctcatagcacgcagcacgctgtatttacgta

SEQ ID NO: 103 - DNA, 60 bp, synthetic construct
  misc_feature 1..60: note = X20282
  source 1..60: mol_type = other DNA
agccagcttaaagagttaaaaatttcatagctaccagaaaggcaacgcaaaatttttttt

SEQ ID NO: 104 - DNA, 60 bp, synthetic construct
  misc_feature 1..60: note = X20283
  source 1..60: mol_type = other DNA
ccctggaaaaaaaattttgcgttgcctttctggtagctatgaaatttttaactctttaag

SEQ ID NO: 105 - DNA, 60 bp, synthetic construct
  misc_feature 1..60: note = X20284
  source 1..60: mol_type = other DNA
tttttaatatatataatgcacacacactaatttagcacgcagcacgctgtatttacgtat

SEQ ID NO: 106 - DNA, 60 bp, synthetic construct
  misc_feature 1..60: note = X20285
  source 1..60: mol_type = other DNA
aattaaatacgtaaatacagcgtgctgcgtgctaaattagtgtgtgtgcattatatatat

SEQ ID NO: 107 - DNA, 60 bp, synthetic construct
  misc_feature 1..60: note = X20286
  source 1..60: mol_type = other DNA
agccagcttaaagagttaaaaatttcatagctatgtggtagaattcaaaagactatgtga

SEQ ID NO: 108 - DNA, 60 bp, synthetic construct
  misc_feature 1..60: note = X20287
  source 1..60: mol_type = other DNA
atggcatcacatagtcttttgaattctaccacatagctatgaaatttttaactctttaag

SEQ ID NO: 109 - DNA, 60 bp, synthetic construct
  misc_feature 1..60: note = X20288
  source 1..60: mol_type = other DNA
ttttaatattgcttttcaattactgttattaaaagcacgcagcacgctgtatttacgtat

SEQ ID NO: 110 - DNA, 60 bp, synthetic construct
  misc_feature 1..60: note = X20289
  source 1..60: mol_type = other DNA
aattaaatacgtaaatacagcgtgctgcgtgcttttaataacagtaattgaaaagcaata

SEQ ID NO: 111 - DNA, 32 bp, synthetic construct
  misc_feature 1..32: note = X20620
  source 1..32: mol_type = other DNA
ggtgattggaatggttatggttccggaatcgc

SEQ ID NO: 112 - DNA, 60 bp, synthetic construct
  misc_feature 1..60: note = X20621
  source 1..60: mol_type = other DNA
ggacgaggcaagctaaacagatctctagacctatatactacatagaaagcaattaaaaga

SEQ ID NO: 113 - DNA, 60 bp, synthetic construct
  misc_feature 1..60: note = X20622
  source 1..60: mol_type = other DNA
ccgtttcttttctttggactatcatgtagtctcctccacctaacaaacccgcaccaacac

SEQ ID NO: 114 - DNA, 32 bp, synthetic construct
  misc_feature 1..32: note = X20623
  source 1..32: mol_type = other DNA
gtcatatggcctcttaacgtggtcctttgtgg

SEQ ID NO: 115 - DNA, 60 bp, synthetic construct
  misc_feature 1..60: note = X20630
  source 1..60: mol_type = other DNA
tttttatcttttaattgctttctatgtagtatataggtctagagatctgtttagcttgcc

SEQ ID NO: 116 - DNA, 60 bp, synthetic construct
  misc_feature 1..60: note = X20631
  source 1..60: mol_type = other DNA
tacttggtgttggtgcgggtttgttaggtggaggagactacatgatagtccaaagaaaag

SEQ ID NO: 117 - DNA, 60 bp, synthetic construct
  misc_feature 1..60: note = X20632
  source 1..60: mol_type = other DNA
tttttatcttttaattgctttctatgtagtatactccacctaacaaacccgcaccaacac

SEQ ID NO: 118 - DNA, 60 bp, synthetic construct
  misc_feature 1..60: note = X20633
  source 1..60: mol_type = other DNA
tacttggtgttggtgcgggtttgttaggtggagtatactacatagaaagcaattaaaaga

SEQ ID NO: 119 - DNA, 36 bp, synthetic construct
  misc_feature 1..36: note = X21123
  source 1..36: mol_type = other DNA
gcgacatgtgatgagattgcatgcacctccacagaa

SEQ ID NO: 120 - DNA, 60 bp, synthetic construct
  misc_feature 1..60: note = X21124
  source 1..60: mol_type = other DNA
ggacgaggcaagctaaacagatctctagacctatctttattctttttattgttgtgaatt

SEQ ID NO: 121 - DNA, 60 bp, synthetic construct
  misc_feature 1..60: note = X21125
  source 1..60: mol_type = other DNA
ccgtttcttttctttggactatcatgtagtctcgcttcaataaaattgttttgtataaat

SEQ ID NO: 122 - DNA, 35 bp, synthetic construct
  misc_feature 1..35: note = X21126
  source 1..35: mol_type = other DNA
ggcagctatctctactatcccgtttagtactatcc

SEQ ID NO: 123 - DNA, 60 bp, synthetic construct
  misc_feature 1..60: note = X21127
  source 1..60: mol_type = other DNA
atattaaattcacaacaataaaaagaataaagataggtctagagatctgtttagcttgcc

SEQ ID NO: 124 - DNA, 60 bp, synthetic construct
  misc_feature 1..60: note = X21128
  source 1..60: mol_type = other DNA
gaactaatttatacaaaacaattttattgaagcgagactacatgatagtccaaagaaaag

SEQ ID NO: 125 - DNA, 59 bp, synthetic construct
  misc_feature 1..59: note = X21133
  source 1..59: mol_type = other DNA
atattaaattcacaacaataaaaagaataaagagcttcaataaaattgttttgtataaa

SEQ ID NO: 126 - DNA, 34 bp, synthetic construct
  misc_feature 1..34: note = X21135
  source 1..34: mol_type = other DNA
gcattgattgtctatcagagcatatcaaggtggt

SEQ ID NO: 127 - DNA, 60 bp, synthetic construct
  misc_feature 1..60: note = X21136
  source 1..60: mol_type = other DNA
ggacgaggcaagctaaacagatctctagacctacggtgactgttgctacttccctatata

SEQ ID NO: 128 - DNA, 60 bp, synthetic construct
  misc_feature 1..60: note = X21137
  source 1..60: mol_type = other DNA
ccgtttcttttctttggactatcatgtagtctcccgtaagcgctattttctttttgttcg

SEQ ID NO: 129 - DNA, 35 bp, synthetic construct
  misc_feature 1..35: note = X21138
  source 1..35: mol_type = other DNA
ggctaggaccccgtaaggaggaaagaataggcaag

SEQ ID NO: 130 - DNA, 60 bp, synthetic construct
  misc_feature 1..60: note = X21139
  source 1..60: mol_type = other DNA
tatatatatatagggaagtagcaacagtcaccgtaggtctagagatctgtttagcttgcc

SEQ ID NO: 131 - DNA, 60 bp, synthetic construct
  misc_feature 1..60: note = X21140
  source 1..60: mol_type = other DNA
tagttacgaacaaaaagaaaatagcgcttacgggagactacatgatagtccaaagaaaag

SEQ ID NO: 132 - DNA, 60 bp, synthetic construct
  misc_feature 1..60: note = X21147
  source 1..60: mol_type = other DNA
tatatatatatagggaagtagcaacagtcaccgccgtaagcgctattttctttttgttcg

SEQ ID NO: 133 - DNA, 60 bp, synthetic construct
  misc_feature 1..60: note = X21148
  source 1..60: mol_type = other DNA
tagttacgaacaaaaagaaaatagcgcttacggcggtgactgttgctacttccctatata

SEQ ID NO: 134 - DNA, 60 bp, synthetic construct
  misc_feature 1..60: note = X21179
  source 1..60: mol_type = other DNA
ttgttttaagctgcaaattaagttgtacaccaagggcgccataaccaaggtatctataga

SEQ ID NO: 135 - DNA, 60 bp, synthetic construct
  misc_feature 1..60: note = X21180
  source 1..60: mol_type = other DNA
tggcggtctatagataccttggttatggcgcccttggtgtacaacttaatttgcagctta

SEQ ID NO: 136 - DNA, 60 bp, synthetic construct
  misc_feature 1..60: note = X21181
  source 1..60: mol_type = other DNA
cttcttctttctttcttgtttttaaagcagcctcgatttttttctaaaccgtggaatatt

SEQ ID NO: 137 - DNA, 60 bp, synthetic construct
  misc_feature 1..60: note = X21182
  source 1..60: mol_type = other DNA
atccgaaatattccacggtttagaaaaaaatcgaggctgctttaaaaacaagaaagaaag

SEQ ID NO: 138 - DNA, 60 bp, synthetic construct
  misc_feature 1..60: note = X21289
  source 1..60: mol_type = other DNA
aaagaaatgtcagagccagaatttcaacaagctaggtctagagatctgtttagcttgcct

SEQ ID NO: 139 - DNA, 60 bp, synthetic construct
  misc_feature 1..60: note = X21290
  source 1..60: mol_type = other DNA
ttaaaatacatcaccttggtcaaacatagcatcgagactacatgatagtccaaagaaaag

SEQ ID NO: 140 - DNA, 60 bp, synthetic construct
  misc_feature 1..60: note = X21291
  source 1..60: mol_type = other DNA
gggacgaggcaagctaaacagatctctagacctagcttgttgaaattctggctctgacat

SEQ ID NO: 141 - DNA, 59 bp, synthetic construct
  misc_feature 1..59: note = X21292
  source 1..59: mol_type = other DNA
ccgtttcttttctttggactatcatgtagtctcgatgctatgtttgaccaaggtgatgt

SEQ ID NO: 142 - DNA, 60 bp, synthetic construct
  misc_feature 1..60: note = X21319
  source 1..60: mol_type = other DNA
ttgttttaagctgcaaattaagttgtacaccaacgatttttttctaaaccgtggaatatt

SEQ ID NO: 143 - DNA, 60 bp, synthetic construct
  misc_feature 1..60: note = X21320
  source 1..60: mol_type = other DNA
atccgaaatattccacggtttagaaaaaaatcgttggtgtacaacttaatttgcagctta

SEQ ID NO: 144 - DNA, 60 bp, synthetic construct
  misc_feature 1..60: note = X21321
  source 1..60: mol_type = other DNA
ttttcagttttggatagatcagttagaaagcttaggctgctttaaaaacaagaaagaaag

SEQ ID NO: 145 - DNA, 60 bp, synthetic construct
  misc_feature 1..60: note = X21322
  source 1..60: mol_type = other DNA
cttcttctttctttcttgtttttaaagcagcctaagctttctaactgatctatccaaaac

SEQ ID NO: 146 - DNA, 60 bp, synthetic construct
  misc_feature 1..60: note = X21507
  source 1..60: mol_type = other DNA
gaactaatttatacaaaacaattttattgaagctctttattctttttattgttgtgaatt

SEQ ID NO: 147 - DNA, 60 bp, synthetic construct
  misc_feature 1..60: note = X21735
  source 1..60: mol_type = other DNA
agccagcttaaagagttaaaaatttcatagctacgatttttttctaaaccgtggaatatt

SEQ ID NO: 148 - DNA, 60 bp, synthetic construct
  misc_feature 1..60: note = X21736
  source 1..60: mol_type = other DNA
atccgaaatattccacggtttagaaaaaaatcgtagctatgaaatttttaactctttaag

SEQ ID NO: 149 - DNA, 31 bp, synthetic construct
  misc_feature 1..31: note = X21754
  source 1..31: mol_type = other DNA
gccaaagtggattctcctactcaagctttgc

SEQ ID NO: 150 - DNA, 60 bp, synthetic construct
  misc_feature 1..60: note = X23319
  source 1..60: mol_type = other DNA
aaagaaatgtcagagccagaatttcaacaagctcgatttttttctaaaccgtggaatatt

SEQ ID NO: 151 - DNA, 60 bp, synthetic construct
  misc_feature 1..60: note = X23320
  source 1..60: mol_type = other DNA
atccgaaatattccacggtttagaaaaaaatcgagcttgttgaaattctggctctgacat

SEQ ID NO: 152 - DNA, 60 bp, synthetic construct
  misc_feature 1..60: note = X23321
  source 1..60: mol_type = other DNA
gtccatgtaaaatgattgctccaatgattgaaagatgctatgtttgaccaaggtgatgta

SEQ ID NO: 153 - DNA, 60 bp, synthetic construct
  misc_feature 1..60: note = X23322
  source 1..60: mol_type = other DNA
ttaaaatacatcaccttggtcaaacatagcatctttcaatcattggagcaatcattttac

SEQ ID NO: 154 - DNA, 60 bp, synthetic construct
  misc_feature 1..60: note = X23408
  source 1..60: mol_type = other DNA
ttttcagttttggatagatcagttagaaagctttagctatgaaatttttaactctttaag

SEQ ID NO: 155 - DNA, 60 bp, synthetic construct
  misc_feature 1..60: note = X23409
  source 1..60: mol_type = other DNA
agccagcttaaagagttaaaaatttcatagctaaagctttctaactgatctatccaaaac

SEQ ID NO: 156 - protein, 730 aa, Saccharomyces cerevisiae
MQDDPENSKLYDLLNSHLDVHGRSNEEPRQTGDSRSQSSGNTGENEEDIAFASGLNGGTFDSMLEALPDDLYFTDFVSPFTAAATTSVTTKTVKDTTPATNHMDDDIAMFDSLATTQPIDIAASNQQNGEIAQLWDFNVDQFNMTPSNSSGSATISAPNSFTSDIPQYNHGSLGNSVSKSSLFPYNSSTSNSNINQPSINNNSNTNAQSHHSFNIYKLQNNNSSSSAMNITNNNNSNNSNIQHPFLKKSDSIGLSSSNTTNSVRKNSLIKPMSSTSLANFKRAASVSSSISNMEPSGQNKKPLIQCFNCKTFKTPLWRRSPEGNTLCNACGLFQKLHGTMRPLSLKSDVIKKRISKKRAKQTDPNIAQNTPSAPATASTSVTTTNAKPIRSRKKSLQQNSLSRVIPEEIIRDNIGNTNNILNVNRGGYNFNSVPSPVLMNSQSYNSSNANFNGASNANLNSNNLMRHNSNTVTPNFRRSSRRSSTSSNTSSSSKSSSRSVVPILPKPSPNSANSQQFNMNMNLMNTTNNVSAGNSVASSPRIISSANFNSNSPLQQNLLSNSFQRQGMNIPRRKMSRNASYSSSFMAASLQQLHEQQQVDVNSNTNTNSNRQNWNSSNSVSTNSRSSNFVSQKPNFDIFNTPVDSPSVSRPSSRKSHTSLLSQQLQNSESNSFISNHKFNNRLSSDSTSPIKYEADVSAGGKISEDNSTKGSSKESSAIADELDWLKFGI

SEQ ID NO: 157 - DNA, 2193 bp, Saccharomyces cerevisiae
  source 1..2193: mol_type = other DNA
atgcaagacgaccccgaaaattcgaagctgtacgacctgctgaatagtcatctggacgtgcatggtcgaagtaatgaagagccgagacaaactggtgacagtaggagccagagtagtggcaacaccggtgaaaacgaggaggatatagcatttgccagtggattaaacggcggcacattcgactcaatgctggaggcactgcccgatgatttatattttacggacttcgtgtctccttttacagcagctgccacgaccagcgtgactactaagacggtcaaggacaccacaccagctaccaatcatatggatgatgatattgcgatgtttgattcacttgccacaactcagcccatcgacatagccgcatccaaccaacaaaatggtgaaattgcacaactttgggactttaacgtggaccaattcaacatgacgcccagcaactcgagcggttcagctactattagtgctcctaacagctttacttccgacataccgcaatacaaccacggttccctcggcaacagcgtctccaaatcctcactgttcccgtataattccagcacgtccaacagcaacatcaaccagccatctatcaataacaactcaaatactaatgcgcagtcccaccattccttcaacatctacaaactacaaaacaacaactcatcttcatccgctatgaacattaccaataataataatagcaacaatagtaatatccagcatccttttctgaagaagagcgattcgataggattatcttcatccaacacaacaaattctgtaagaaaaaactcacttatcaagccaatgtcgtccacgtccctggccaatttcaaaagagctgcctcagtatcttccagtatatccaatatggaaccatcaggacaaaataaaaaacctctgatacaatgtttcaattgtaaaactttcaagacaccgctttggaggagaagcccagaggggaatactctttgcaatgcctgcggtcttttccagaaattacatggtaccatgaggccattatccttaaaatcggacgttatcaaaaagaggatttcaaagaagagagccaaacaaacggacccaaacattgcacaaaatactccaagtgcacctgcaactgcctcaacttcagtaaccactacaaatgctaaacccatacgatcgaggaaaaaatcactacaacaaaactctttatctagagtgatacctgaagaaatcattagagacaacatcggtaatactaataatatccttaatgtaaataggggaggctataacttcaactcagtcccctccccggtcctcatgaacagccaatcgtataatagtagtaacgcaaattttaatggagcaagcaatgcaaatttgaattctaataacttaatgcgtcacaattcgaacactgttactcctaattttagaaggtcttcaagacgaagtagtacttcatcgaacacctcaagttccagtaaatcttcatccagatctgttgttccgatattaccaaaaccttcacctaatagcgctaattcacagcagttcaacatgaacatgaacctaatgaacacaacaaataatgtaagtgcaggaaatagtgtcgcatcctcaccaagaattatatcgtccgcaaactttaactcaaatagtcctctacagcagaatctattatcaaattctttccaacgtcaaggaatgaatataccaagaagaaagatgtcgcgcaatgcatcgtactcctcatcgtttatggctgcgtctttgcaacaactgcacgaacagcaacaagtggacgtgaattccaacacaaacacgaattcgaatagacagaattggaattcaagcaatagcgtttcaacaaattcaagatcatcaaattttgtctctcaaaagccaaattttgatatttttaatactcctgtagattcaccgagtgtctcaagaccttcttcaagaaaatcacatacctcattgttatcacaacaattgcagaactcggagtcgaattcgtttatctcaaatcacaaatttaacaatagattatcaagtgactctacttcacctataaaatatgaagcagatgtgagtgcaggcggaaagatcagtgaggataattccacaaaaggatcttctaaagaaagttcagcaattgctgacgaattggattggttaaaatttggtatatga

SEQ ID NO: 158 - DNA, 753 bp, Saccharomyces cerevisiae
  source 1..753: mol_type = other DNA
atgcctttgaccacaaaacctttatctttgaaaatcaacgccgctctattcgatgttgacggtaccatcatcatctctcaaccagccattgctgctttctggagagatttcggtaaagacaagccttacttcgatgccgaacacgttattcacatctctcacggttggagaacttacgatgccattgccaagttcgctccagactttgctgatgaagaatacgttaacaagctagaaggtgaaatcccagaaaagtacggtgaacactccatcgaagttccaggtgctgtcaagttgtgtaatgctttgaacgccttgccaaaggaaaaatgggctgtcgccacctctggtacccgtgacatggccaagaaatggttcgacattttgaagatcaagagaccagaatacttcatcaccgccaatgatgtcaagcaaggtaagcctcacccagaaccatacttaaagggtagaaacggtttgggtttcccaattaatgaacaagacccatccaaatctaaggttgttgtctttgaagacgcaccagctggtattgctgctggtaaggctgctggctgtaaaatcgttggtattgctaccactttcgatttggacttcttgaaggaaaagggttgtgacatcattgtcaagaaccacgaatctatcagagtcggtgaatacaacgctgaaaccgatgaagtcgaattgatctttgatgactacttatacgctaaggatgacttgttgaaatggtaa

SEQ ID NO: 159 - protein, 250 aa, Saccharomyces cerevisiae
MPLTTKPLSLKINAALFDVDGTIIISQPAIAAFWRDFGKDKPYFDAEHVIHISHGWRTYDAIAKFAPDFADEEYVNKLEGEIPEKYGEHSIEVPGAVKLCNALNALPKEKWAVATSGTRDMAKKWFDILKIKRPEYFITANDVKQGKPHPEPYLKGRNGLGFPINEQDPSKSKVVVFEDAPAGIAAGKAAGCKIVGIATTFDLDFLKEKGCDIIVKNHESIRVGEYNAETDEVELIFDDYLYAKDDLLKW

SEQ ID NO: 160 - DNA, 753 bp, Saccharomyces cerevisiae
  source 1..753: mol_type = other DNA
atgggattgactactaaacctctatctttgaaagttaacgccgctttgttcgacgtcgacggtaccattatcatctctcaaccagccattgctgcattctggagggatttcggtaaggacaaaccttatttcgatgctgaacacgttatccaagtctcgcatggttggagaacgtttgatgccattgctaagttcgctccagactttgccaatgaagagtatgttaacaaattagaagctgaaattccggtcaagtacggtgaaaaatccattgaagtcccaggtgcagttaagctgtgcaacgctttgaacgctctaccaaaagagaaatgggctgtggcaacttccggtacccgtgatatggcacaaaaatggttcgagcatctgggaatcaggagaccaaagtacttcattaccgctaatgatgtcaaacagggtaagcctcatccagaaccatatctgaagggcaggaatggcttaggatatccgatcaatgagcaagacccttccaaatctaaggtagtagtatttgaagacgctccagcaggtattgccgccggaaaagccgccggttgtaagatcattggtattgccactactttcgacttggacttcctaaaggaaaaaggctgtgacatcattgtcaaaaaccacgaatccatcagagttggcggctacaatgccgaaacagacgaagttgaattcatttttgacgactacttatatgctaaggacgatctgttgaaatggtaa

SEQ ID NO: 161 - protein, 250 aa, Saccharomyces cerevisiae
MGLTTKPLSLKVNAALFDVDGTIIISQPAIAAFWRDFGKDKPYFDAEHVIQVSHGWRTFDAIAKFAPDFANEEYVNKLEAEIPVKYGEKSIEVPGAVKLCNALNALPKEKWAVATSGTRDMAQKWFEHLGIRRPKYFITANDVKQGKPHPEPYLKGRNGLGYPINEQDPSKSKVVVFEDAPAGIAAGKAAGCKIIGIATTFDLDFLKEKGCDIIVKNHESIRVGGYNAETDEVEFIFDDYLYAKDDLLKW

SEQ ID NO: 162 - DNA, 1548 bp, Saccharomycopsis fibuligera
  source 1..1548: mol_type = other DNA
atgatcagattgaccgttttcttgaccgctgtttttgctgctgttgcttcttgtgttccagttgaattggataagagaaacaccggtcatttccaagcttattctggttataccgttgctagatctaacttcacccaatggattcatgaacaaccagctgtttcttggtactacttgttgcaaaacatcgattacccagaaggtcaattcaaatctgctaaaccaggtgttgttgttgcttctccatctacatctgaaccagattacttctaccaatggactagagataccgctattaccttcttgtccttgattgctgaagttgaagatcattctttctccaacactaccttggctaaggttgtcgaatattacatttccaacacctacaccttgcaaagagtttctaatccatccggtaacttcgattctccaaatcatgatggtttgggtgaacctaagttcaacgttgatgatactgcttatacagcttcttggggtagaccacaaaatgatggtccagctttgagagcttacgctatttctagatacttgaacgctgttgctaagcacaacaacggtaaattattattggccggtcaaaacggtattccttattcttctgcttccgatatctactggaagattattaagccagacttgcaacatgtttctactcattggtctacctctggttttgatttgtgggaagaaaatcaaggtactcatttcttcaccgctttggttcaattgaaggctttgtcttacggtattccattgtctaagacctacaatgatccaggtttcacttcttggttggaaaaacaaaaggatgccttgaactcctacattaactcttccggtttcgttaactctggtaaaaagcacatcgttgaatctccacaattgtcatctagaggtggtttggattctgctacttatattgctgccttgatcacccatgatatcggtgatgatgatacttacaccccattcaatgttgataactcctacgttttgaactccttgtattacctattggtcgacaacaagaacagatacaagatcaacggtaactacaaagctggtgctgctgttggtagatatcctgaagatgtttacaacggtgttggtacttctgaaggtaatccatggcaattggctactgcttatgctggtcaaactttttacaccttggcctacaattccttgaagaacaagaagaacttggtcatcgaaaagttgaactacgacttgtacaactccttcattgctgatttgtccaagattgattcttcctacgcttctaaggattctttgactttgacctacggttccgataactacaagaacgttatcaagtccttgttgcaattcggtgactcattcttgaaggttttgttggatcacatcgatgacaacggtcaattgactgaagaaatcaacagatacaccggttttcaagctggtgcagtttctttgacttggtcatctggttctttgttgtctgctaatagagccagaaacaagttgatcgaattattgtaa

SEQ ID NO: 163 - protein, 515 aa, Saccharomycopsis fibuligera
MIRLTVFLTAVFAAVASCVPVELDKRNTGHFQAYSGYTVARSNFTQWIHEQPAVSWYYLLQNIDYPEGQFKSAKPGVVVASPSTSEPDYFYQWTRDTAITFLSLIAEVEDHSFSNTTLAKVVEYYISNTYTLQRVSNPSGNFDSPNHDGLGEPKFNVDDTAYTASWGRPQNDGPALRAYAISRYLNAVAKHNNGKLLLAGQNGIPYSSASDIYWKIIKPDLQHVSTHWSTSGFDLWEENQGTHFFTALVQLKALSYGIPLSKTYNDPGFTSWLEKQKDALNSYINSSGFVNSGKKHIVESPQLSSRGGLDSATYIAALITHDIGDDDTYTPFNVDNSYVLNSLYYLLVDNKNRYKINGNYKAGAAVGRYPEDVYNGVGTSEGNPWQLATAYAGQTFYTLAYNSLKNKKNLVIEKLNYDLYNSFIADLSKIDSSYASKDSLTLTYGSDNYKNVIKSLLQFGDSFLKVLLDHIDDNGQLTEEINRYTGFQAGAVSLTWSSGSLLSANRARNKLIELL

(19) United States
(12) Patent Application Publication
(10) Pub. No.: US 2024/0360484 A1
(43) Pub. Date: Oct. 31, 2024

(54) METHODS FOR REGULATING NITROGEN METABOLISM DURING THE PRODUCTION OF ETHANOL FROM CORN BY METABOLICALLY ENGINEERED YEAST STRAINS

(71) Applicant: Lallemand Hungary Liquidity Management LLC, Budapest (HU)

(72) Inventors: Aaron Argyros, White River Junction, VT (US); Trisha Barrett, Bradford, VT (US)

(21) Appl. No.: 18/443,045

(22) Filed: Feb. 15, 2024

Related U.S. Application Data
(63) Continuation of application No. 17/592,915, filed on Feb. 4, 2022, now Pat. No. 11,946,089.
Application No. 17/592,915 is a continuation of application No. 16/570,881, filed on Sep. 13, 2019, now Pat. No. 11,274,319.
Application No. 16/570,881 is a continuation of application No. 14/771,831, filed on Sep. 1, 2015, now Pat. No. 10,604,772.
(60) Provisional application No. 61/800,323, filed on Mar. 15, 2013.

(51) Int. Cl. C12P 7/06; C12N 9/00; C12N 9/02; C12N 9/04; C12N 9/06; C12N 9/10; C12N 9/34; C12N 9/50; C12N 9/60; C12N 15/52; C12P 7/10

(52) U.S. Cl. CPC C12P 7/06; C12N 9/0004; C12N 9/0006; C12N 9/0008; C12N 9/0016; C12N 9/1029; C12N 9/2428; C12N 9/50; C12N 9/60; C12N 9/93; C12N 15/52; C12Y 104/01002; C12Y 104/01004; C12Y 104/01014; C12Y 603/01002; C12Y 603/04006; C12P 7/10; C12Y 101/01001; C12Y 101/01002; C12Y 102/0101; C12Y 197/01004; C12Y 203/01054; C12Y 302/01003; C12Y 304/23041; Y02E 50/10

(57) ABSTRACT

The present invention provides for a mechanism to reduce glycerol production and increase nitrogen utilization and ethanol production of recombinant microorganisms. One aspect of this invention relates to strains of S. cerevisiae with reduced glycerol productivity that get a kinetic benefit from higher nitrogen concentration without sacrificing ethanol yield. A second aspect of the invention relates to—metabolic modifications resulting in altered transport and/or intracellular metabolism of nitrogen sources present in corn mash.

### CROSS REFERENCE TO RELATED APPLICATIONS

The present application is a continuation of U.S. application Ser. No. 17/592,915, filed Feb. 2, 2022, which is a continuation of U.S. application Ser. No. 16/570,881, filed Sep. 13, 2019, which is a continuation of U.S. application Ser. No. 14/771,831, filed Sep. 1, 2015, which is a U.S. national stage of International Application No. PCT/US2014/025460, filed Mar. 13, 2014, which claims priority benefit to U.S. Provisional Application No. 61/800,323, filed Mar. 15, 2013, each of which is hereby incorporated by reference in its entirety.

### SEQUENCE LISTING

The content of the electronically submitted sequence listing (Name: 4200_0450004_SequenceListing_ST26.xml; Size: 266,896 bytes; and Date of Creation: Jul. 16, 2024), filed with the application, is incorporated herein by reference in its entirety.

### BACKGROUND OF THE INVENTION

Energy conversion, utilization and access underlie many of the great challenges of our time, including those associated with sustainability, environmental quality, security, and poverty. New applications of emerging technologies are required to respond to these challenges. Biotechnology, one of the most powerful of the emerging technologies, can give rise to important new energy conversion processes. Plant biomass and derivatives thereof are a resource for the biological conversion of energy to forms useful to humanity.

Among forms of plant biomass, both grain-based biomass and lignocellulosic biomass (collectively “biomass”) are well-suited for energy applications. Each feedstock has advantages and disadvantages. For example, because of its large-scale availability, low cost, and environmentally benign production lignocellulosic biomass has gained attention as a viable feed source for biofuel production. In particular, many energy production and utilization cycles based on cellulosic biomass have near-zero greenhouse gas emissions on a life-cycle basis.

However, grain-based feed stocks are more readily converted to fuels by existing microorganisms, although grain-based feed stock is more expensive than lignocellulosic feed stock and conversion to fuel competes with alternative uses for the grain.

Biomass processing schemes involving enzymatic or microbial hydrolysis commonly involve four biologically mediated transformations: (1) the production of saccharolytic enzymes (cellulases and hemicellulases); (2) the hydrolysis of carbohydrate components present in pretreated biomass to sugars; (3) the fermentation of hexose sugars (e.g., glucose, mannose, and galactose); and (4) the fermentation of pentose sugars (e.g., xylose and arabinose). These four transformations can occur in a single step in a process configuration called consolidated bioprocessing (“CBP”), which is distinguished from other less highly integrated configurations in that it does not involve a dedicated process step for cellulase and/or hemicellulase production.

CBP offers the potential for lower cost and higher efficiency than processes featuring dedicated cellulase production. The benefits result in part from avoided capital costs, substrate and other raw materials, and utilities associated with cellulase production. In addition, several factors support the realization of higher rates of hydrolysis, and hence reduced reactor volume and capital investment using CBP, including enzyme-microbe synergy and the use of thermophilic organisms and/or complexed cellulase systems. Moreover, cellulose-adherent cellulolytic microorganisms are likely to compete successfully for products of cellulose hydrolysis with non-adhered microbes, e.g., contaminants. Successful competition of desirable microbes increases the stability of industrial processes based on microbial cellulose utilization. Progress in developing CBP-enabling microorganisms is being made through two strategies: engineering naturally occurring cellulolytic microorganisms to improve product-related properties, such as yield and titer; and engineering non-cellulolytic organisms that exhibit high product yields and titers to express a heterologous cellulase and hemicellulase system enabling cellulose and hemicellulose utilization.

One way to meet the demand for ethanol production is to convert sugars found in biomass, i.e., materials such as agricultural wastes, corn hulls, corncobs, cellulosic materials, and the like to produce ethanol. Efficient biomass conversion in large-scale industrial applications requires a microorganism that is able to tolerate high concentrations of sugar and ethanol, and which is able to ferment more than one sugar simultaneously.

Bakers' yeast (Saccharomyces cerevisiae) is the preferred microorganism for the production of ethanol (Hahn-Hagerdal, B., et al., Adv. Biochem. Eng. Biotechnol. 73, 53-84 (2001)). Attributes in favor of this microbe are (i) high productivity at close to theoretical yields (0.51 g ethanol produced/g glucose used), (ii) high osmo- and ethanol tolerance, (iii) natural robustness in industrial processes, also (iv) being generally regarded as safe (GRAS) due to its long association with wine and bread making, and beer brewing. Furthermore, S. cerevisiae exhibits tolerance to inhibitors commonly found in hydrolysates resulting from biomass pretreatment. Exemplary metabolic pathways for the production of ethanol are depicted in FIG. 1. However, S. cerevisiae does not naturally break down components of cellulose, nor does it efficiently use pentose sugars.

Glycerol is a metabolic end-product of native yeast ethanolic fermentation (FIG. 1). During anaerobic growth on carbohydrates, production of ethanol and carbon dioxide is redox neutral, while the reactions that create cell biomass and associated carbon dioxide are more oxidized relative to carbohydrates. The production of glycerol, which is more reduced relative to carbohydrates, functions as an electron sink to off-set cell biomass formation, so that overall redox neutrality is conserved. This is essential from a theoretical consideration of conservation of mass, and in practice strains unable to produce glycerol are unable (or only very poorly able) to grow under anaerobic conditions.

There is a strong commercial incentive not to produce glycerol as a byproduct because it represents lost ethanol yield. In industrial corn ethanol fermentations, this yield loss can be up to 6% of theoretical, for a market of ˜14 billion gallons/yr. At selling price of $2.50/gal, this is a total market value of $2 B/yr.

Strategies from the literature to address this problem include decreasing glycerol formation by engineering ammonia fixation to function with NADH instead of NADPH via up-regulation of GLN1, encoding glutamine synthetase, or GLT1, encoding glutamate synthase with deletion of GDH1, encoding the NADPH-dependent glutamate dehydrogenase. (Nissen, T. L., et al., Metabolic Engineering 2: 69-77 (2000)). Another strategy engineering cells to produce excess NADPH during glycolysis via expression of a NADPH linked glyceraldehyde-3-phosphate dehydrogenase. (Bro, C., et al., Metabolic Engineering 8: 102-111 (2006)). Another strategy contained a deletion of GDH1, and over-expression of glutamate synthase (GLT1) and glutamine synthase (GLN1), which also resulted in reduced glycerol formation. However, growth rates and biomass formation were well below the control strain and improvements on the initial performance have not been demonstrated. Additionally, industrially relevant yields, titers and fermentation rates were never demonstrated. (U.S. Pat. No. 7,018,829). Another strategy describes deletion of only GDH1 without overexpression of GDH2 or GLT1/GLN1. However, the strategy was dependent on the use of an industrial polyploid yeast strain capable of tolerating high ethanol concentrations. It is noted in the patent that GDH1 was the only deletion, and that there are no heterologous DNA sequences in the genome. Additionally, the maximum reduction in glycerol production seen was 12.04%, and the technology was not demonstrated on an industrially relevant substrate (U.S. Pat. No. 7,935,514). Most glycerol reduction strategies either only partially reduce the requirement for glycerol formation, or create a by-product other than ethanol. The present invention overcomes the shortcomings of these other strategies.

Corn mash contains free amino nitrogen. However the amount is too low to enable yeast biomass formation sufficient to meet the needs of the process. Nitrogen is added to industrial corn ethanol fermentations to promote yeast growth, most commonly in the form of urea and ammonia. Excess nitrogen can improve the fermentation kinetics of conventional yeasts; however ethanol yields are often lower due to excess biomass and glycerol formation. Typically, urea is added to industrial corn fermentations in concentrations that range from 500 ppm to 1000 ppm.

Yeast take up and assimilate ammonium as its preferred nitrogen source, followed by amino acids, and finally urea (FIGS. 2-4) (extensively reviewed by Lungdhal et al., Genetics 190: 885-929 (2012)). The mechanism of nitrogen catabolite repression (NCR) control is established by transcription factors which control the expression of ammonium, amino acid and urea transporters. These transcription factors also control expression of proteins responsible for degradation and assimilation of nitrogen containing molecules. It has been shown that de-repression of non-preferred nitrogen source assimilation pathways can improve fermentation kinetics (Salmon, J. M., and Barre, P., Appl. Environ. Microbiol. 64:3831-3837 (1998)); however, effects on ethanol productivity were not measured.

S. cerevisiae contains three known ammonium transporters, MEP1, MEP2 and MEP3. MEP1 and MEP2 are high affinity transporters while MEP3 is a low affinity transporter. S. cerevisiae breaks down urea through the enzymatic action of a urea-amido lyase (EC 6.3.4.6). This activity is encoded by the enzyme DUR1/2 in S. cerevisiae (FIGS. 2-4). Overexpression of DUR1/2 in wine yeasts has been shown to enhance urea degradation rates during fermentation of grape must (Coulon, J., et al., Am. J. Enol. Vitic. 57:2 (2006)). There are two known urea transporters in S. cerevisiae, DUR3 and DUR4 (FIGS. 2-4). It has been shown that overexpression of DUR3 resulted in improved urea degradation rates during wine fermentation (Dahabieh, M. S., et al., Am. J. Enol. Vitic. 60:4 (2009)). U.S. Patent Publ. No. 2011/0129566 describes the expression of DUR1/2 and DUR3 in wine yeasts.

Industrial corn mash substrates contain as much as 3% protein (w/v); however, much of the amino acid content contained in these proteins is unavailable to S. cerevisiae. Expression of one or more proteases would release amino acids that could serve as a nitrogen source for yeast. Additionally, the use of amino acids as a nitrogen source for S. cerevisiae in corn ethanol fermentations would improve yield through a reduction in the surplus NADH generated from in vivo amino acid synthesis during anaerobic growth.

Guo et al. engineered S. cerevisiae to express a heterologous protease for the purpose of improving ethanol yield (Guo, Z-p, et al., Enzyme and Microbial Technology 48: 148-154 (2011)). However, the work was conducted in a wild type yeast background that had not been previously engineered for reduced glycerol formation, and the activity of the expressed endoprotease primarily breaks protein into short polypeptides which are not transported by S. cerevisiae.

One aspect of the present invention relates to improved fermentation performance through co-expression of an exoprotease to release single amino acids. Additionally, corn kernel protein is primarily a class of storage proteins known as zeins. Zeins have been shown to be recalcitrant to hydrolysis by many proteases and it is possible that expression of zein specific proteases would result in improved proteolysis. Thus, another aspect of the present invention relates to expressing zein-specific proteases to improve corn protein hydrolysis and amino acid utilization by the yeast.

Amino acids are transported by a large family of amino acid permeases. One aspect of this invention relates to deregulation or over-expression of a specific or general amino acid permease to complement protease expression or metabolic engineering by improving the uptake rate of free amino acids released during proteolysis. For example, expression of the general amino acid permease GAP1 is negatively regulated by AUA1. One aspect of this invention relates to the deletion of AUA1 or over expression of GAP1 that could result in improved amino acid uptake rates.

PCT/US2012/032443, which is incorporated herein by reference, teaches a method of eliminating glycerol formation through the production of formate. The formate production pathway can also be combined with strains engineered for reduced activity of the native glycerol production pathway. These combinations can be designed such that strains are built with different degrees of glycerol reduction as shown in FIG. 5. Several embodiments of the current invention relate to a combination of those or related genetic modifications described in PCT/US2012/032443, with additional genetic modifications that are designed to alter nitrogen transport and assimilation.

One aspect of this invention relates to strains of S. cerevisiae with reduced glycerol production that get a kinetic benefit from higher nitrogen concentration without sacrificing ethanol yield. A second aspect of the invention relates to metabolic modifications resulting in altered transport and/or intracellular metabolism of nitrogen sources present in corn mash.

### BRIEF SUMMARY OF THE INVENTION

Some embodiments are direct to a recombinant microorganism comprising: at least one engineered genetic modification that leads to the up-regulation or down-regulation of one or more native and/or heterologous enzymes that function in one or more ethanol production pathways; at least one engineered genetic modification that leads to the down-regulation of an enzyme in a glycerol-production pathway; and at least one engineered genetic modification that leads to the up-regulation or down-regulation of an enzyme in a nitrogen-assimilation pathway.

In some embodiments of the invention, the down-regulated enzyme in the nitrogen-assimilation pathway is glutamate dehydrogenase (Gdh) (EC 1.4.1.4).

In some embodiments of the invention, the microorganism further comprises least one genetic modification that leads to the up-regulation of an enzyme in a nitrogen-assimilation pathway.

In some embodiments of the invention, the up-regulated enzyme in the nitrogen-assimilation pathway is at least one enzyme selected from the group consisting of glutamate dehydrogenase (Gdh) (EC 1.4.1.2), glutamate synthase (Glt) (EC 1.4.1.14), and glutamine synthase (Gln) (EC 6.3.1.2). In some embodiments of the invention, the up-regulated enzyme in the nitrogen-assimilation pathway is a native ammonium transporter. In some embodiments of the invention, the up-regulated enzyme in the nitrogen-assimilation pathway is a MEP protein from the genus Saccharomyces. In some embodiments of the invention, the up-regulated enzyme in the nitrogen assimilation pathway is a urea-amido lyase (EC 6.3.4.6). In some embodiments of the invention, the up-regulated enzyme in the nitrogen assimilation pathway is a urea transporter. In some embodiments of the invention, the up-regulated enzyme in the nitrogen assimilation pathway is Gln3.

In some embodiments of the invention, the enzyme in the glycerol-production pathway is encoded by at least one enzyme selected from the group consisting of: a glycerol-3-phosphate dehydrogenase 1 polynucleotide (GPD1) (EC 1.1.1.8), a glycerol-3-phosphate dehydrogenase 1 polypeptide (Gpd1) (EC 1.1.1.8), a glycerol-3-phosphate dehydrogenase 2 polynucleotide (GPD2) (EC 1.1.1.8), a glycerol-3-phosphate dehydrogenase 2 polypeptide (Gpd2) (EC 1.1.1.8), a glycerol-3-phosphate phosphatase 1 polynucleotide (GPP1) (EC 3.1.3.21), a glycerol-3-phosphate phosphatase polypeptide 1 (Gpp1) (EC 3.1.3.21), a glycerol-3-phosphate phosphatase 2 polynucleotide (GPP2) (EC 3.1.3.21), and a glycerol-3-phosphate phosphatase polypeptide 2 (Gpp2) (EC 3.1.3.21).

In some embodiments of the invention, up-regulated enzyme that acts in an ethanol production pathway is pyruvate formate lyase (EC 2.3.1.54). In some embodiments of the invention, the up-regulated enzyme that acts in the ethanol production pathway is pyruvate formate lyase activating enzyme (EC 1.91.1.4).

In some embodiments of the invention, the up-regulated enzyme that acts in the ethanol production pathway is bifunctional acetaldehyde-alcohol dehydrogenase selected from a group of enzymes having both of the following Enzyme Commission Numbers: EC 1.2.1.10 and 1.1.1.1.

In some embodiments of the invention, the up-regulated enzyme that acts in the ethanol production pathway is an NADPH-dependent bifunctional acetaldehyde-alcohol dehydrogenase selected from a group of enzymes having both of the following Enzyme Commission Numbers: EC 1.2.1.10 and 1.1.1.2.

In some embodiments, the microorganism further comprises a down-regulation in one or more native enzymes encoded by a formate dehydrogenase enzyme selected from the group consisting of: EC 1.2.1.43 and EC 1.2.1.2.

In some embodiments of the invention, the recombinant microorganism further comprises a heterologous GPD1 polynucleotide operably linked to a native GPD2 promoter. In some embodiments of the invention, the recombinant microorganism further comprises a heterologous GPD2 polynucleotide operably linked to a native GPD1 promoter.

In some embodiments of the invention, the microorganism further comprises an up-regulation or down-regulation of a regulatory element. In some embodiments the regulatory element is selected from the group consisting of: Ure2 and Aua1.

In some embodiments of the invention, the microorganism further comprises at least one additional up-regulated enzyme. In some embodiments of the invention, the additional up-regulated enzyme is a glucoamylase enzyme with EC number 3.2.1.3. In some embodiments of the invention, the additional up-regulated enzyme is a permease. In some embodiments of the invention, the additional up-regulated enzyme is a protease with EC number: 3.4.23.41.

In some embodiments of the invention, the up-regulated or down-regulated enzymes are under the control of a heterologous promoter. In some embodiments of the invention, the heterologous promoter is selected from a group consisting of: TEF2 (SEQ ID NO: 58), HXT7 (SEQ ID NO: 59), ADH1 (SEQ ID NO: 60), and TPI (SEQ ID NO: 61).

In some embodiments, the microorganism is a yeast. In some embodiments, the yeast is from the genus Saccharomyces. In some embodiments, the yeast is Saccharomyces cerevisiae. In some embodiments, the microorganism produces ethanol at a higher yield than an otherwise identical microorganism lacking the genetic modifications. In some embodiments, the microorganism produces an ethanol titer about 1% to about 10% more than an otherwise identical microorganism lacking the genetic modifications. In some embodiments, the microorganism produces an ethanol titer of at least about 125 g/L.

In some embodiments, the microorganism produces glycerol at a lower yield than an otherwise identical microorganism lacking the genetic modifications. In some embodiments, the microorganism produces a glycerol titer of about 10 to about 100% less than an otherwise identical microorganism lacking the genetic modifications.

In some embodiments, the invention relates to a composition comprising any recombinant microorganism herein, and a carbon-containing feedstock.

Some embodiments of the invention are directed to a method of producing a fermentation product using any composition herein, wherein the recombinant microorganism is capable of fermenting the carbon containing feedstock to yield the fermentation product.

Some embodiments of the invention are directed to a method of producing a fermentation product comprising: any composition provided herein; contacting the composition with a carbon containing feedstock, wherein the recombinant microorganism is capable of fermenting the carbon containing feedstock to yield the fermentation product; and, optionally recovering the fermentation production

Some embodiments of the invention are directed to a method of producing ethanol comprising: providing any recombinant microorganism herein; culturing the recombinant microorganism in the presence of a carbon containing feedstock for sufficient time to produce ethanol; and, optionally, extracting the ethanol.

Some embodiments of the invention are directed to a co-culture comprising at least two host cells, wherein one of the host cells comprises any recombinant microorganism herein; and another host cell that is genetically distinct from the recombinant microorganism.

Some embodiments of the invention are directed to a recombinant microorganism comprising: down-regulated Gpd1, down-regulated Gpd2, down-regulated Fdh1, down-regulated Fdh2, down-regulated Gdh1, up-regulated AdhE, up-regulated pyruvate formate lyase, an up-regulated pyruvate formate lyase-activating enzyme, GPD1 under the control of the GPD2 promoter, GPD2 under the control of the GPD1 promoter, and up-regulated Gdh2.

Some embodiments of the invention are directed to a recombinant microorganism comprising: down-regulated Gpd1, down-regulated Gpd2, down-regulated Fdh1, down-regulated Fdh2, down-regulated Gdh1, up-regulated AdhE, up-regulated pyruvate formate lyase, an up-regulated pyruvate formate lyase-activating enzyme, GPD1 under the control of the GPD2 promoter, GPD2 under the control of the GPD1 promoter, up-regulated Glt1 and up-regulated Gln1.

Some embodiments of the invention are directed to a recombinant microorganism comprising: down-regulated Gpd1, down-regulated Fdh1, down-regulated Fdh2, down-regulated Gdh1, up-regulated AdhE, up-regulated pyruvate formate lyase, an up-regulated pyruvate formate lyase-activated enzyme, GPD1 under the control of the GPD2 promoter, GPD2 under the control of the GPD1 promoter, up-regulated Glt1 and up-regulated Gln1.

Some embodiments of the invention are directed to a recombinant microorganism comprising: down-regulated Gpd2, down-regulated Fdh1, down-regulated Fdh2, down-regulated Gdh1, up-regulated AdhE, up-regulated pyruvate formate lyase, and an up-regulated pyruvate formate lyase-activating enzyme.

Some embodiments of the invention are directed to a recombinant microorganism comprising: down-regulated Gpd1, down-regulated Fdh1, down-regulated Fdh2, down-regulated Gdh1, up-regulated AdhE, up-regulated pyruvate formate lyase, an up-regulated pyruvate formate lyase-activating enzyme, and GPD2 under the control of the GPD1 promoter.

Some embodiments of the invention are directed to a recombinant microorganism comprising: down-regulated Gpd1, down-regulated Fdh1, down-regulated Fdh2, down-regulated Gdh1, up-regulated AdhE, up-regulated pyruvate formate lyase, and an up-regulated pyruvate formate lyase-activating enzyme.

Some embodiments of the invention are directed to a recombinant microorganism comprising: down-regulated Gpd1, down-regulated Fdh1, down-regulated Fdh2, up-regulated AdhE, up-regulated pyruvate formate lyase, an up-regulated pyruvate formate lyase-activating enzyme, upregulated-DUR/12, and GPD2 under the control of the GPD1 promoter.

Some embodiments of the invention are directed to a recombinant microorganism comprising: down-regulated Gpd1, down-regulated Fdh1, down-regulated Fdh2, up-regulated AdhE, up-regulated pyruvate formate lyase, an up-regulated pyruvate formate lyase-activating enzyme, and up-regulated-DUR/12.

Some embodiments of the invention are directed to a recombinant microorganism comprising: down-regulated Gpd1, down-regulated Gpd2, down-regulated Fdh1, down-regulated Fdh2, down-regulated Ure2, up-regulated AdhE, up-regulated pyruvate formate lyase, an up-regulated pyruvate formate lyase-activating enzyme, GPD1 under the control of the GPD2 promoter, and GPD2 under the control of the GPD1 promoter.

Some embodiments of the invention are directed to a recombinant microorganism comprising: down-regulated Gpd1, down-regulated Gpd2, down-regulated Fdh1, down-regulated Fdh2, up-regulated AdhE, up-regulated pyruvate formate lyase, an up-regulated pyruvate formate lyase-activating enzyme, up-regulated GDH2, GPD1 under the control of the GPD2 promoter, and GPD2 under the control of the GPD1 promoter.

### BRIEF DESCRIPTION OF THE DRAWINGS/FIGURES

FIG. 1 depicts simplified carbon and redox pathways utilized by wildtype S. cerevisiae during anaerobic growth. Ethanol formation is redox neutral while cell biomass formation generates net NADH, which is balanced by glycerol formation.

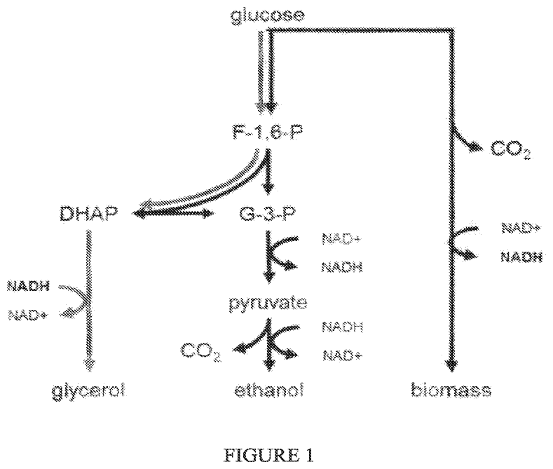

FIG. 2 depicts urea transport and intracellular catabolism. The enzymes Dur3 and Dur4 are known transporters of urea. Once inside the cell, urea is broken down into 2 ammonia molecules and 2 carbon dioxide molecules in a reaction catalyzed by Dur1,2.

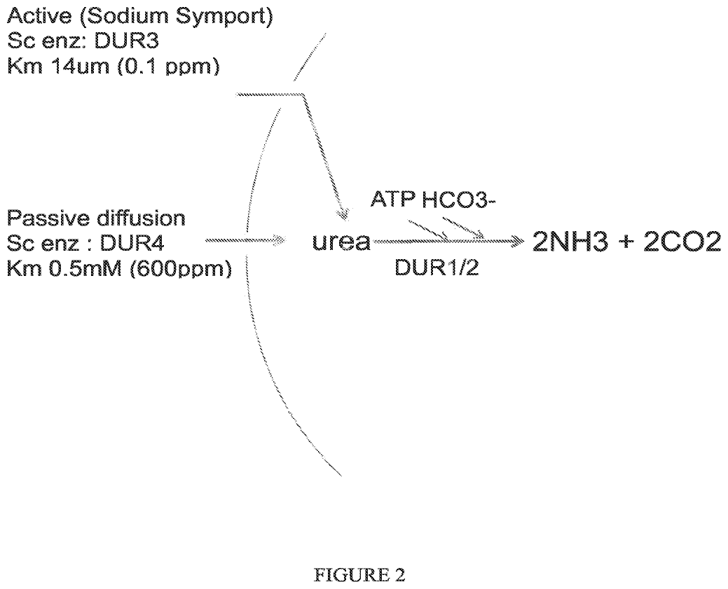

FIG. 3 depicts the process by which an unmodified S. cerevisiae assimilates urea.

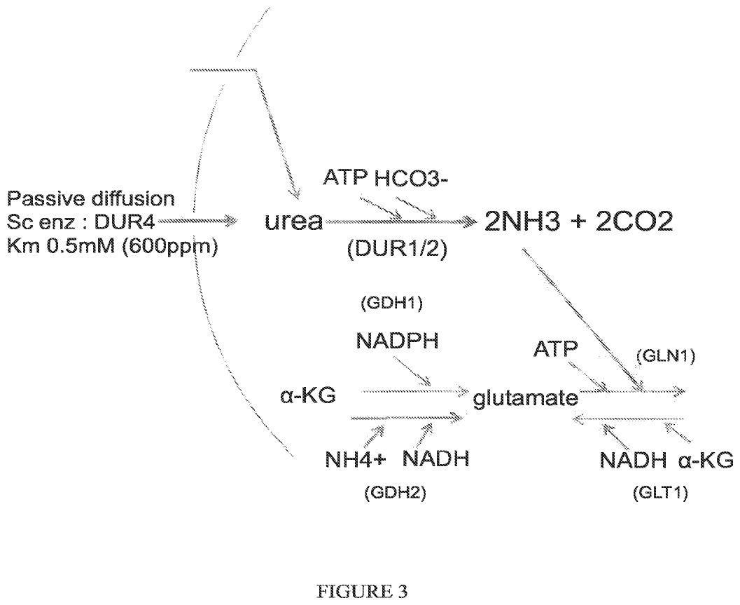

FIG. 4 depicts process by which a genetically modified glycerol reduction strain which contains a deletion of Gdh1 assimilates urea.

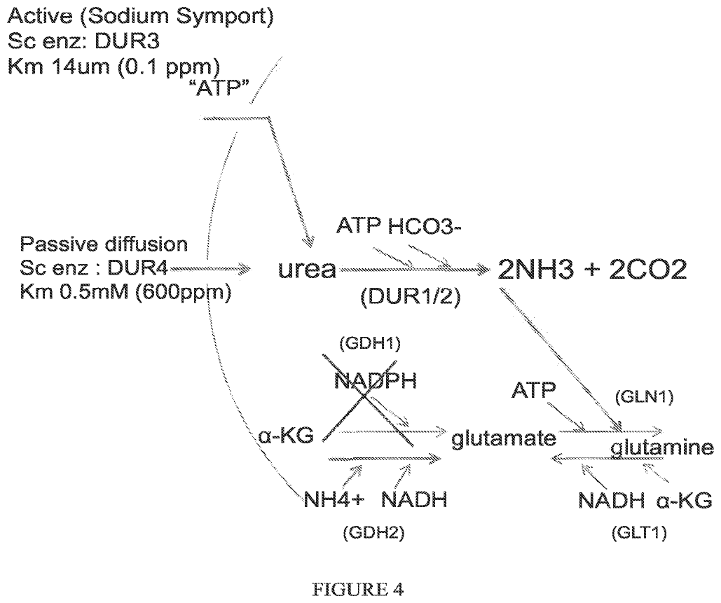

FIG. 5 depicts the glycerol titers of the wild type and glycerol reduction strains containing the formate pathway (M2390 (wildtype), M3465, M3467, M3469, and M3624 are depicted). This data shows total glycerol present in corn mash which contained 7 g/l glycerol prior to fermentation.

FIG. 6 depicts a schematic diagram of the MA0631 insertion cassette.

FIG. 7 depicts a schematic diagram of the MA0425 insertion cassette.

FIG. 8 depicts a schematic diagram of the MA0426 insertion cassette.

FIG. 9 depicts a schematic diagram of the MA0888 insertion cassette.

FIG. 10 depicts a schematic diagram of the MA0837 insertion cassette.

FIG. 11 depicts a schematic diagram of the MA0616 insertion cassette.

FIG. 12 depicts a schematic diagram of the MA0616.1 insertion cassette.

FIG. 13 depicts a schematic diagram of the MA0615 insertion cassette.

FIG. 14 depicts a schematic diagram of the MA0615.1 insertion cassette.

FIG. 15 depicts a schematic diagram of the MA0622 insertion cassette.

FIG. 16 depicts a schematic diagram of the MA0622.1 insertion cassette.

FIG. 17 depicts a schematic diagram of the MA0580 insertion cassette.

FIG. 18 depicts a schematic diagram of the MA0581 insertion cassette.

FIG. 19 depicts a schematic diagram of the MA0582 insertion cassette.

FIG. 20 depicts a schematic diagram of the MA0583 insertion cassette.

FIG. 21 depicts a schematic diagram of the MA0584 insertion cassette.

FIG. 22 depicts a schematic diagram of the MA0585 insertion cassette.

FIG. 23 depicts a schematic diagram of the MA0617 insertion cassette.

FIG. 24 depicts a schematic diagram of the MA0617.1 insertion cassette.

FIG. 25 depicts a schematic diagram of the MA0434 insertion cassette.

FIG. 26 depicts a schematic diagram of the MA0434.2 insertion cassette.

FIG. 27 depicts a schematic diagram of the MA0434.3 insertion cassette.

FIG. 28 depicts a schematic diagram of the MA0434.4 insertion cassette.

FIG. 29 depicts a schematic diagram of the MA0434.5 insertion cassette.

FIG. 30 depicts a schematic diagram of the MA0454.14 insertion cassette.

FIG. 31 depicts a schematic diagram of the MA0464 insertion cassette.

FIG. 32 depicts a schematic diagram of the MA0464.1 insertion cassette.

FIG. 33 depicts a schematic diagram of the MA0464.2 insertion cassette.

FIG. 34 depicts a schematic diagram of the MA0464.3 insertion cassette.

FIG. 35 depicts a schematic diagram of the MA0464.4 insertion cassette.

FIG. 36 depicts a schematic diagram of the MA0464.5 insertion cassette.

FIG. 37 depicts a schematic diagram of the MA0465.1 insertion cassette.

FIG. 38 depicts a schematic diagram of the MA0467 insertion cassette.

FIG. 39 depicts a schematic diagram of the MA0467.1 insertion cassette.

FIG. 40 depicts a schematic diagram of the MA0467.2 insertion cassette.

FIG. 41 depicts a schematic diagram of the MA0467.3 insertion cassette.

FIG. 42 depicts a schematic diagram of the MA0467.4 insertion cassette.

FIG. 43 depicts a schematic diagram of the MA0881 insertion cassette.

FIG. 44 depicts a schematic diagram of the MA0881.1 insertion cassette.

FIG. 45 depicts a plasmid map for pMU2873.

FIG. 46 depicts a plasmid map for pMU2879.

FIG. 47 depicts a plasmid map for pMU2908.

FIG. 48 depicts a plasmid map for pMU2909.

FIG. 49 depicts a plasmid map for pMU2911.

FIG. 50 depicts a plasmid map for pMU2913.

FIG. 51 depicts a plasmid map for pMU3409.

FIG. 52 depicts a plasmid map for pMU3410.

FIG. 53 depicts a plasmid map for pMU3411.

FIG. 54 depicts a plasmid map for pMU3459.

FIG. 55 depicts a plasmid map for pMU3460.

FIG. 56 depicts a plasmid map for pMU3461.

FIG. 57 depicts a plasmid map for pMU3463.

FIG. 58 depicts a plasmid map for pMU3464.

FIG. 59 depicts a plasmid map for pMU3465.

FIG. 60 depicts a plasmid map for pMU3466.

FIG. 61 depicts a plasmid map for pMU3468.

FIG. 62 depicts a plasmid map for pMU3471.

FIG. 63 depicts a plasmid map for pMU3472.

FIG. 64 depicts a plasmid map for pMU3473.

FIG. 65 depicts a plasmid map for pMU3475.

FIG. 66 depicts a plasmid map for pMU3605.

FIG. 67 depicts a plasmid map for pMU3606.

FIG. 68 depicts a plasmid map for pMU3607.

FIG. 69 depicts the final ethanol titers measured following fermentation of 31% solids corn mash in wildtype cells (M2390), a glycerol reduction strain containing the formate pathway (M3624), and 2 strains with modification of the ammonium assimilation pathway (M4117, which contains a deletion of gdh1 and an over-expression of Gdh2, and M4118, which contains a deletion of gdh1 and an over-expression of Glt1 and Gln1).

FIG. 70 depicts the ethanol titers measured following fermentation of 31% solids corn mash for glycerol reduction strains containing the formate pathway (M3465, M3467, M3469) that additionally have a deletion of gdh1 (M4400, M4401, M4402). M2390 was a parental control.

FIG. 71 depicts the ethanol titers measured following fermentation of 31% solids corn mash for M2390, M3467, M3469, M4427 (M3467 parent strain: expression of DUR1/2 driven by the TEF2 promoter), M4428 (M3467 parent strain: expression of DUR1/2 driven by the HXT7 promoter), M4429 (M3467 parent strain: expression of DUR1/2 driven by the ADH1 promoter), M4430 (M3467 parent strain: expression of DUR1/2 driven by the HXT7/TEF2 promoters), M4431 (M3469 parent strain: expression of DUR1/2 driven by the TEF2 promoter), M4432 (M3469 parent strain: expression of DUR1/2 driven by the HXT7 promoter), M4433 (M3469 parent strain: expression of DUR1/2 driven by the ADH1 promoter), and M4434 (M3469 parent strain: expression of DUR1/2 driven by the HXT7/TEF2 promoters)

FIG. 72 depicts the ethanol titers measured following fermentation of 31% solids corn mash for M2390, M3624, M4406, and M4407.

FIG. 73 depicts the ethanol titers produced after 68 hrs fermentation in mini-vials for strains M2390, M3624, M4117, M5841, M5842, M5843, and M5844.

FIG. 74 depicts the glycerol titers produced after 68 hrs fermentation in mini-vials for strains M2390, M3624, M4117, M5841, M5842, M5843, and M5844.

### DETAILED DESCRIPTION OF THE INVENTION

### Definitions

Unless defined otherwise, all technical and scientific terms used herein have the same meaning as commonly understood to one of ordinary skill in the art of microbial metabolic engineering. Although methods and materials similar or equivalent to those described herein can be used in the practice of the disclosed methods and compositions, exemplary methods, devices and materials are described herein.

The embodiments described, and references in the specification to “one embodiment”, “an embodiment”, “an example embodiment”, etc., indicate that the embodiments described can include a particular feature, structure, or characteristic, but every embodiment does not necessarily include the particular feature, structure, or characteristic. Moreover, such phrases are not necessarily referring to the same embodiment. Further, when a particular feature, structure, or characteristic is described in connection with an embodiment, it is understood that it is within the knowledge of one skilled in the art to effect such feature, structure, or characteristic in connection with other embodiments whether or not explicitly described.

The description of “a” or “an” item herein may refer to a single item or multiple items. It is understood that wherever embodiments are described herein with the language “comprising,” otherwise analogous embodiments described in terms of “consisting of” and/or “consisting essentially of” are also provided. Thus, for example, reference to “a polynucleotide” includes a plurality of such polynucleotides and reference to “the microorganism” includes reference to one or more microorganisms, and so forth.

The term “heterologous” is used in reference to a polynucleotide or a gene not normally found in the host organism. “Heterologous” includes up-regulated or down-regulated endogenous genes. “Heterologous” also includes a native coding region, or portion thereof, that is reintroduced into the source organism in a form that is different from the corresponding native gene, e.g., not in its natural location in the organism's genome. “Heterologous” also includes any gene that has been modified and placed into an organism. A heterologous gene may include a native coding region that is a portion of a chimeric gene including a non-native regulatory region that is reintroduced into the native host or modifications to the native regulatory sequences that affect the expression level of the gene. Foreign genes can comprise native genes inserted into a non-native organism, or chimeric genes. A heterologous polynucleotide, gene, polypeptide, or an enzyme may be derived or isolated from any source, e.g., eukaryotes, prokaryotes, viruses, or synthetic polynucleotide fragments, and includes up-regulated endogenous genes.

The terms “gene(s)” or “polynucleotide” or “nucleic acid” or “polynucleotide sequence(s)” are intended to include nucleic acid molecules, e.g., polynucleotides which include an open reading frame encoding a polypeptide, and can further include non-coding regulatory sequences, and introns. In addition, the terms are intended to include one or more genes that map to a functional locus. Also, the terms are intended to include a specific gene for a selected purpose. The gene may be endogenous to the host cell or may be recombinantly introduced into the host cell, e.g., as a plasmid maintained episomally or a plasmid (or fragment thereof) that is stably integrated into the genome. In addition to the plasmid form, a gene may, for example, be in the form of linear DNA or RNA. The term “gene” is also intended to cover multiple copies of a particular gene, e.g., all of the DNA sequences in a cell encoding a particular gene product. A “gene” refers to an assembly of nucleotides that encode a polypeptide, and includes cDNA and genomic DNA nucleic acids. “Gene” also refers to a nucleic acid fragment that expresses a specific protein, including intervening sequences (introns) between individual coding segments (exons), as well as regulatory sequences preceding (5′ non-coding sequences) and following (3′ non-coding sequences) the coding sequence. “Native” or “endogenous” refers to a gene as found in nature with its own regulatory sequences.

A “nucleic acid,” “polynucleotide,” or “nucleic acid molecule” is a polymeric compound comprised of covalently linked subunits called nucleotides. Nucleic acid includes polyribonucleic acid (RNA) and polydeoxyribonucleic acid (DNA), both of which may be single-stranded or double-stranded. DNA includes cDNA, genomic DNA, synthetic DNA, and semi-synthetic DNA.

An “isolated nucleic acid molecule” or “isolated nucleic acid fragment” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA).

The term “expression” is intended to include the expression of a gene at least at the level of mRNA production, generally subsequently translated into a protein product. The term “expression,” refers to the transcription and stable accumulation of sense (mRNA) or antisense RNA derived from the nucleic acid fragment of the invention. Expression may also refer to translation of mRNA into a polypeptide.

As used herein, an “expression vector” is a vector capable of directing the expression of genes to which it is operably linked.

A “vector,” e.g., a “plasmid” or “YAC” (yeast artificial chromosome) refers to an extrachromosomal element often carrying one or more genes that are not part of the central metabolism of the cell, and is usually in the form of a circular double-stranded DNA molecule. Such elements may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. Preferably, the plasmids or vectors of the present invention are stable and self-replicating.

The term “integrated” as used herein refers to genetic elements that are placed, through molecular biology techniques, into a chromosome of a host cell. For example, genetic elements can be placed into the chromosomes of the host cell as opposed to in a vector such as a plasmid carried by the host cell. Methods for integrating genetic elements into the genome of a host cell are well known in the art and include homologous recombination.

The term “domain” as used herein refers to a part of a molecule or structure that shares common physical or chemical features, for example hydrophobic, polar, globular, helical domains or properties, e.g., a DNA binding domain or an ATP binding domain. Domains can be identified by their homology to conserved structural or functional motifs. Examples of cellobiohydrolase (CBH) domains include the catalytic domain (CD) and the cellulose binding domain (CBD).

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength. Hybridization and washing conditions are well known and exemplified, e.g., in Sambrook, J., Fritsch, E. F. and Maniatis, T. MOLECULAR CLONING: A LABORATORY MANUAL, Second Edition, Cold Spring Harbor Laboratory Press, Cold Spring Harbor (1989), particularly Chapter 11 and Table 11.1 therein (hereinafter “Maniatis”, entirely incorporated herein by reference). The conditions of temperature and ionic strength determine the “stringency” of the hybridization. Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions. One set of conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. For more stringent conditions, washes are performed at higher temperatures in which the washes are identical to those above except for the temperature of the final two 30 min washes in 0.2×SSC, 0.5% SDS are increased to 60° C. Another set of highly stringent conditions uses two final washes in 0.1×SSC, 0.1% SDS at 65° C. An additional set of highly stringent conditions are defined by hybridization at 0.1×SSC, 0.1% SDS, 65° C. and washed with 2×SSC, 0.1% SDS followed by 0.1×SSC, 0.1% SDS.

Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (see, e.g., Maniatis at 9.50-9.51). For hybridizations with shorter nucleic acids, i.e., oligonucleotides, the position of mismatches becomes more important, and the length of the oligonucleotide determines its specificity (see, e.g., Maniatis, at 11.7-11.8). In one embodiment the length for a hybridizable nucleic acid is at least about 10 nucleotides. Preferably a minimum length for a hybridizable nucleic acid is at least about 15 nucleotides; more preferably at least about 20 nucleotides; and most preferably the length is at least 30 nucleotides. Furthermore, the skilled artisan will recognize that the temperature and wash solution salt concentration may be adjusted as necessary according to factors such as length of the probe.

The term “percent identity”, as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences.

As known in the art, “similarity” between two polypeptides is determined by comparing the amino acid sequence and conserved amino acid substitutes thereto of the polypeptide to the sequence of a second polypeptide.

“Identity” and “similarity” can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5.

Suitable nucleic acid sequences or fragments thereof (isolated polynucleotides of the present invention) encode polypeptides that are at least about 70% to about 75% identical to the amino acid sequences reported herein, at least about 80%, about 85%, or about 90% identical to the amino acid sequences reported herein, or at least about 95%, about 96%, about 97%, about 98%, about 99%, or about 100% identical to the amino acid sequences reported herein. Suitable nucleic acid fragments are at least about 70%, about 75%, or about 80% identical to the nucleic acid sequences reported herein, at least about 80%, about 85%, or about 90% identical to the nucleic acid sequences reported herein, or at least about 95%, about 96%, about 97%, about 98%, about 99%, or about 100% identical to the nucleic acid sequences reported herein. Suitable nucleic acid fragments not only have the above identities/similarities but typically encode a polypeptide having at least 50 amino acids, at least 100 amino acids, at least 150 amino acids, at least 200 amino acids, or at least 250 amino acids.

A DNA or RNA “coding region” is a DNA or RNA molecule which is transcribed and/or translated into a polypeptide in a cell in vitro or in vivo when placed under the control of appropriate regulatory sequences. “Suitable regulatory regions” refer to nucleic acid regions located upstream (5′ non-coding sequences), within, or downstream (3′ non-coding sequences) of a coding region, and which influence the transcription, RNA processing or stability, or translation of the associated coding region. Regulatory regions may include promoters, translation leader sequences, RNA processing site, effector binding site and stem-loop structure. The boundaries of the coding region are determined by a start codon at the 5′ (amino) terminus and a translation stop codon at the 3′ (carboxyl) terminus. A coding region can include, but is not limited to, prokaryotic regions, cDNA from mRNA, genomic DNA molecules, synthetic DNA molecules, or RNA molecules. If the coding region is intended for expression in a eukaryotic cell, a polyadenylation signal and transcription termination sequence will usually be located 3′ to the coding region.

An “isoform” is a protein that has the same function as another protein but which is encoded by a different gene and may have small differences in its sequence.

A “paralogue” is a protein encoded by a gene related by duplication within a genome.

An “orthologue” is gene from a different species that has evolved from a common ancestral gene by speciation. Normally, orthologues retain the same function in the course of evolution as the ancestral gene.

“Open reading frame” is abbreviated ORF and means a length of nucleic acid, either DNA, cDNA or RNA, that comprises a translation start signal or initiation codon, such as an ATG or AUG, and a termination codon and can be potentially translated into a polypeptide sequence.

“Promoter” refers to a DNA fragment capable of controlling the expression of a coding sequence or functional RNA. In general, a coding region is located 3′ to a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of cellular development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity. A promoter is generally bounded at its 3′ terminus by the transcription initiation site and extends upstream (5′ direction) to include the minimum number of bases or elements necessary to initiate transcription at levels detectable above background. Within the promoter will be found a transcription initiation site (conveniently defined for example, by mapping with nuclease Si), as well as protein binding domains (consensus sequences) responsible for the binding of RNA polymerase.

A coding region is “under the control” of transcriptional and translational control elements in a cell when RNA polymerase transcribes the coding region into mRNA, which is then trans-RNA spliced (if the coding region contains introns) and translated into the protein encoded by the coding region.

“Transcriptional and translational control regions” are DNA regulatory regions, such as promoters, enhancers, terminators, and the like, that provide for the expression of a coding region in a host cell. In eukaryotic cells, polyadenylation signals are control regions.

The term “operably associated” refers to the association of nucleic acid sequences on a single nucleic acid fragment so that the function of one is affected by the other. For example, a promoter is operably associated with a coding region when it is capable of affecting the expression of that coding region (i.e., that the coding region is under the transcriptional control of the promoter). Coding regions can be operably associated to regulatory regions in sense or antisense orientation.

As used herein, the term “anaerobic” refers to an organism, biochemical reaction or process that is active or occurs under conditions of an absence of gaseous O₂.

“Anaerobic conditions” are defined as conditions under which the oxygen concentration in the fermentation medium is too low for the microorganism to use as a terminal electron acceptor. Anaerobic conditions may be achieved by sparging a fermentation medium with an inert gas such as nitrogen until oxygen is no longer available to the microorganism as a terminal electron acceptor. Alternatively, anaerobic conditions may be achieved by the microorganism consuming the available oxygen of fermentation until oxygen is unavailable to the microorganism as a terminal electron acceptor.

“Aerobic metabolism” refers to a biochemical process in which oxygen is used as a terminal electron acceptor to convert energy, typically in the form of ATP, from carbohydrates. Aerobic metabolism typically occurs, for example, via the electron transport chain in mitochondria in eukaryotes, wherein a single glucose molecule is metabolized completely into carbon dioxide in the presence of oxygen.

In contrast, “anaerobic metabolism” refers to a biochemical process in which oxygen is not the final acceptor of electrons generated. Anaerobic metabolism can be divided into anaerobic respiration, in which compounds other than oxygen serve as the terminal electron acceptor, and substrate level phosphorylation, in which no exogenous electron acceptor is used and products of an intermediate oxidation state are generated via a “fermentative pathway.”

In “fermentative pathways”, the amount of NAD(P)H generated by glycolysis is balanced by the consumption of the same amount of NAD(P)H in subsequent steps. For example, in one of the fermentative pathways of certain yeast strains, NAD(P)H generated through glycolysis donates its electrons to acetaldehyde, yielding ethanol. Fermentative pathways are usually active under anaerobic conditions but may also occur under aerobic conditions, under conditions where NADH is not fully oxidized via the respiratory chain.

As used herein, the term “end-product” refers to a chemical compound that is not or cannot be used by a cell, and so is excreted or allowed to diffuse into the extracellular environment. Common examples of end-products from anaerobic fermentation include, but are not limited to, ethanol, acetic acid, formic acid, lactic acid, hydrogen and carbon dioxide.

As used herein, “cofactors” are compounds involved in biochemical reactions that are recycled within the cells and remain at approximately steady state levels. Common examples of cofactors involved in anaerobic fermentation include, but are not limited to, NAD⁺ and NADP⁺. In metabolism, a cofactor can act in oxidation-reduction reactions to accept or donate electrons. When organic compounds are broken down by oxidation in metabolism, their energy can be transferred to NAD⁺ by its reduction to NADH, to NADP⁺ by its reduction to NADPH, or to another cofactor, FAD⁺, by its reduction to FADH₂. The reduced cofactors can then be used as a substrate for a reductase.

As used herein, a “pathway” is a group of biochemical reactions that together can convert one compound into another compound in a step-wise process. A product of the first step in a pathway may be a substrate for the second step, and a product of the second step may be a substrate for the third, and so on. Pathways of the present invention include, but are not limited to, the pyruvate metabolism pathway the lactate production pathway, the ethanol production pathway, the glycerol-production pathway, the nitrogen assimilation pathway, and the ammonium assimilation pathway.

The term “recombination” or “recombinant” refers to the physical exchange of DNA between two identical (homologous), or nearly identical, DNA molecules. Recombination can be used for targeted gene deletion or to modify the sequence of a gene. The term “recombinant microorganism” and “recombinant host cell” are used interchangeably herein and refer to microorganisms that have been genetically modified to express or over-express endogenous polynucleotides, or to express heterologous polynucleotides, such as those included in a vector, or which have a modification in expression of an endogenous gene.

By “expression modification” it is meant that the expression of the gene, or level of a RNA molecule or equivalent RNA molecules encoding one or more polypeptides or polypeptide subunits, or activity of one or more polypeptides or polypeptide subunits is up regulated or down-regulated, such that expression, level, or activity, is greater than or less than that observed in the absence of the modification.

In one aspect of the invention, genes or particular polynucleotide sequences are partially, substantially, or completely deleted, silenced, inactivated, or down-regulated in order to inactivate the enzymatic activity they encode. Complete deletions provide maximum stability because there is no opportunity for a reverse mutation to restore function. Alternatively, genes can be partially, substantially, or completely deleted, silenced, inactivated, or down-regulated by insertion, deletion, removal or substitution of nucleic acid sequences that disrupt the function and/or expression of the gene.

As used herein, the term “down-regulate” includes the deletion or mutation of a genetic sequence, or insertion of a disrupting genetic element, coding or non-coding, such that the production of a gene product is lessened by the deletion, mutation, or insertion. It includes a decrease in the expression level (i.e., molecular quantity) of an mRNA or protein. “Delete” or “deletion” as used herein refers to a removal of a genetic element such that a corresponding gene is completely prevented from being expressed. In some embodiments, deletion refers to a complete gene deletion. Down-regulation can also occur by engineering the repression of genetic elements by chemical or other environmental means, for example by engineering a chemically-responsive promoter element (or other type of conditional promoter) to control the expression of a desired gene product. Down-regulation can also occur through use of a weak promoter.

As used herein, the term “up-regulate” includes the insertion, reintroduction, mutation, or increased expression of a genetic sequence, such that the production of a gene product is increased by the insertion, reintroduction, or mutation. It includes an increase in the expression level (i.e., molecular quantity) of an mRNA or protein. “Insert” or “insertion” as used herein refers to an introduction of a genetic element such that a corresponding gene is expressed. Up-regulation can also occur by causing the increased expression of genetic elements through an alteration of the associated regulatory sequence. Up-regulation can occur by engineering the expression of genetic elements by chemical or other environmental means, for example by engineering a chemically-responsive promoter element (or other type of conditional promoter) to control the expression of a desired gene product. Up-regulation can also occur through use of a strong promoter.

As used herein, the term “glycerol-production pathway” refers to the collection of biochemical pathways that produce glycerol from DHAP. Components of the pathway consist of all substrates, cofactors, byproducts, intermediates, end-products, and enzymes in the pathway.

As used herein, the term “ethanol production pathway” refers the collection of biochemical pathways that produce ethanol from a carbohydrate source. Components of the pathway consist of all substrates, cofactors, byproducts, intermediates, end-products, and enzymes in the pathway.

As used herein, the term “nitrogen assimilation pathway” refers to the collection of biochemical pathways that result in the formation of organic nitrogen containing compounds from inorganic nitrogen compounds. Components of the pathway consist of all substrates, cofactors, byproducts, intermediates, end-products, and enzymes in the pathway.

As used herein, the term “ammonium assimilation pathway” refers to the collection of biochemical pathways that assimilate ammonia or ammonium (NH₄⁺) into glutamate and/or glutamine. The ammonium assimilation pathway is part of the larger nitrogen assimilation pathway. Components of the pathway consist of all substrates, cofactors, byproducts, intermediates, end-products, and enzymes in the pathway.

As used herein, the term “glycolysis” or “glycolytic pathway” refers to the canonical pathway of basic metabolism in which a sugar such as glucose is broken down into more oxidized products, converting energy and compounds required for cell growth. Components of the pathway consist of all substrates, cofactors, byproducts, intermediates end-products, and enzymes in the pathway.

As used herein, the term “alcohol dehydrogenase” or “ADH” is intended to include the enzymes that catalyze the conversion of ethanol into acetylaldehyde. Very commonly, the same enzyme catalyzes the reverse reaction from acetaldehyde to ethanol, which is the direction more relevant to fermentation. Alcohol dehydrogenase includes those enzymes that correspond to EC 1.1.1.1 and 1.1.1.2 and exemplified by the enzymes disclosed in GenBank Accession No. U49975.

As used herein, the term “aldehyde dehydrogenase”, “ALD” or “ALDH” is intended to include the enzymes that catalyze the oxidation of aldehydes. Aldehyde dehydrogenase enzymes include “acetaldehyde dehydrogenase”, which catalyzes the conversion of acetaldehyde into acetyl-CoA. Very commonly, the same enzyme catalyzes the reverse reaction from acetyl-CoA to acetaldehyde, which is the direction more relevant to fermentation. Aldehyde dehydrogenase includes those enzymes that correspond to EC 1.2.1.3, 1.2.1.4 and 1.2.1.10.

As used herein, the term “glycerol-3-phosphate dehydrogenase” or “GPD” is intended to include those enzymes capable of converting dihydroxyacetone phosphate to glycerol-3-phosphate. GPD includes those enzymes that correspond to EC 1.1.1.8. In some embodiments, the GPD is GPD1 and/or GPD2 from S. cerevisiae (GDP1: SEQ ID NO: 4 and 5, GDP2: SEQ ID NO: 6 and 7).

As used herein, the term “glycerol-3-phosphate phosphatase” or “GPP” is intended to include those enzymes capable of converting glycerol-1-phosphate to glycerol. Glycerol-3-phosphate is intended to include those enzymes that correspond to EC 3.1.3.21. (GPP1: SEQ ID NO: 158 and 159, GPP2: SEQ ID NO 160 and 161)

As used herein, the term “formate dehydrogenase” or “FDH” is intended to include those enzymes capable of converting formate to bicarbonate (carbon dioxide). Formate dehydrogenase includes those enzymes that correspond to EC 1.2.1.43 and EC 1.2.1.2. In some embodiments, the FDH is from S. cerevisiae (FDH1: SEQ ID NO: 1 and 2, FDH2: SEQ ID NO: 3).

As used herein, the term “bifunctional” is intended to include enzymes that catalyze more than one biochemical reaction step. A specific example of a bifunctional enzyme used herein is an enzyme (adhE) that catalyzes both the alcohol dehydrogenase and acetaldehyde dehydrogenase reactions, and includes those enzymes that correspond to EC 1.2.1.10 and 1.1.1.1. In some embodiments, the bifunctional acetaldehyde-alcohol dehydrogenase is from B. adolescentis (adhE: SEQ ID NO: 12 and 13). In some embodiments, the bifunctional enzyme is a NADPH specific bifunctional acetaldehyde-alcohol dehydrogenase, and includes those enzymes that correspond to EC 1.2.1.10 and 1.1.1.2. In some embodiments, the NADPH specific bifunctional acetaldehyde-alcohol dehydrogenase is from L. mesenteroides (SEQ ID NO: 14 and 15) or O. oenii (SEQ ID NO: 16 and 17).

As used herein, the term “pyruvate formate lyase” or “PFL” is intended to include the enzymes capable of converting pyruvate to formate and acetyl-CoA. PFL includes those enzymes that correspond to EC 2.3.1.54 and exemplified by SEQ ID NO: 8 and 9.

As used herein, the term “PFL-activating enzymes” is intended to include those enzymes capable of aiding in the activation of PFL. PFL-activating enzymes include those enzymes that correspond to EC 1.97.1.4 and are exemplified by SEQ ID NO: 10 and 11.

As used herein, the term “glutamate dehydrogenase”, “GDH”, or “GLDH” is intended to include those enzymes that convert glutamate to α-ketoglutarate, as well as those enzymes that catalyze the reverse reaction. The glutamate dehydrogenase can be NADPH-dependent (e.g. GDH1 or GDH3 in S. cerevisiae). The glutamate dehydrogenase can be NADH-dependent (e.g. GDH2 in S. cerevisiae). Glutamate dehydrogenases include those enzymes that correspond to EC 1.4.1.2 and EC 1.4.1.4. Glutamate dehydrogenases include those enzymes that correspond to accession numbers: M10590, S66436, S66039.1, U12980, NP_015020, NP_010066, S66039.1 and AAC04972. In some embodiments, the glutamate dehydrogenase is from S. cerevisiae (GDH1: SEQ ID NOs: 25 and 25; GDH2: SEQ ID NOs: 26 and 27; GDH3: SEQ ID NOs: 30 and 31.) or N. crassa (GDH2: SEQ ID NOs: 28 29).

As used herein, the term “glutamate synthase” or “GLT” is intended to include those enzymes that convert L-glutamine and 2-oxoglutarate to L-glutamate, as well as those enzymes that catalyze the reverse reaction. Glutamate synthases include those enzymes that correspond to EC 1.4.1.14 and EC 1.4.1.13. In some embodiments, the glutamate synthase is GLT1 from S. cerevisiae (SEQ ID NOs: 32 and 33; accession numbers: X89221 and NP_010110.1).

As used herein, the term “glutamine synthase”, “glutamine synthetase”, or “GLN” is intended to include those enzymes that convert glutamate to glutamine. Glutamine synthases include those enzymes that correspond to EC 6.3.1.2. In some embodiments, the glutamine synthase is GLN1 from S. cerevisiae (SEQ ID NOs: 34 and 35; accession numbers: M65157 and NP_015360.2).

As used herein, the term “urea-amido lyase” is intended to include those enzymes that convert urea to urea-1-carboxylate. Urea-amido lyases include those enzymes that correspond to EC 6.3.4.6. In some embodiments, the urea-amido lyase is DUR1/2 (DUR1,2) from S. cerevisiae (SEQ ID NOs: 36 and 37; accession numbers: M64926 and NP_009767.1)

As used herein, the term “urea transporter” is a membrane protein that transports urea across a cellular membrane. In some embodiments, the urea transporter is Dur3 or Dur4 from S. cerevisiae (DUR3: SEQ ID NOs: 38 and 39; accession numbers: AY693170 and NP_011847.1).

As used herein, the term “protease” is any enzyme that hydrolyzes the peptide bonds between amino acids together in a protein. An exoprotease is a protease that breaks the peptide bonds of terminal amino acids in a protein. An endoprotease is a protease that breaks the peptide bonds of non-terminal amino acids in a protein. Proteases include those enzymes that correspond to EC 3.4.23.41. Proteases include those enzymes that correspond to accession numbers: NP_001151278, NP_001150196, NP_001148706, NCU00338, XP_001908191, XP_369812, EU970094.1, NM_001156724, NM_001155234.1, XP_957809.2, XM_001908156.1, and XM_003717209.1. In some embodiments, the protease is from Z. mays (SEQ ID NOs: 40-45), N. crassa (SEQ ID NOs: 46-47), P. anserine (SEQ ID NOs: 48-49), or M oryzae (SEQ ID NOs: 50-51).

As used herein, the term “glucoamylase” or “γ-amylase” refers to an amylase that acts on α-1,6-glycosidic bonds. Glucoamylases include those enzymes that correspond to EC 3.2.1.3. In some embodiments, the glucoamylase is S. fibuligera glucoamylase (glu-0111-CO) (SEQ ID NO: 162 and 163).

As used herein, the term “permease” refers to a membrane transport protein that facilitates the diffusion of a molecule through the use of passive transport in or out of a cell. In some embodiments, the permease is the amino acid permease GAP1 from S. cerevisiae. (SEQ ID NO: 52 and 53).

As used herein, the term “ammonium transporter” refers to permeases, and is intended to include the enzymes that are involved in the transport of ammonium and ammonia, and are exemplified by the S. cerevisiae MEP1, MEP2 and MEP3 enzymes (MEP 1: SEQ ID NOs: 18 and 19; MEP2: SEQ ID NOs: 20 and 21; MEP3: SEQ ID NOs: 22 and 23). Ammonium transporters include those enzymes that correspond to accession numbers: X77608, X83608, AY692775, NP_011636.3, NP_014257.1, and NP_015464.1.

As used herein, the term “URE2” refers to transcription factor known in the art by that name that represses the nitrogen catabolism of glutamate by controlling the transcription factor. URE2 is a regulator of GLN3. In some embodiments, the URE2 is from S. cerevisiae (SEQ ID NOs: 54 and 55).

As used herein, “AUA1” refers to a transcription factor known in the art by that name which is required for the negative regulation of Gap1. In some embodiments, the AUA1 is from S. cerevisiae (SEQ ID NOs: 56 and 57).

As used herein, “GLN3” refers to a transcription factor known in the art by that name that activates genes that are regulated by nitrogen catabolite metabolism. In some embodiments, the GLN3 is from S. cerevisiae (SEQ ID NOs: 156 and 157). The term “feedstock” is defined as a raw material or mixture of raw materials supplied to a microorganism or fermentation process from which other products can be made. For example, a carbon source, such as biomass or the carbon compounds derived from biomass are a feedstock for a microorganism that produces a product in a fermentation process. A feedstock can contain nutrients other than a carbon source.

Biomass can include any type of biomass known in the art or described herein. The terms “lignocellulosic material,” “lignocellulosic substrate” and “cellulosic biomass” mean any type of carbon containing feed stock including woody biomass, such as recycled wood pulp fiber, sawdust, hardwood, softwood, grasses, sugar-processing residues, agricultural wastes, such as, but not limited to, rice straw, rice hulls, barley straw, corn cobs, cereal straw, wheat straw, canola straw, oat straw, oat hulls, corn fiber, stover, succulents, agave, or any combination thereof.

The term “yield” is defined as the amount of product obtained per unit weight of raw material and may be expressed as gram product per gram substrate (g/g). Yield may be expressed as a percentage of the theoretical yield. “Theoretical yield” is defined as the maximum amount of product that can be generated per a given amount of substrate as dictated by the stoichiometry of the metabolic pathway used to make the product. For example, the theoretical yield for one typical conversion of glucose to ethanol is 0.51 g EtOH per 1 g glucose. As such, a yield of 4.8 g ethanol from 10 g of glucose would be expressed as 94% of theoretical or 94% theoretical yield.

The term “titer” is defined as the strength of a solution or the concentration of a substance in solution. For example, the titer of a product in a fermentation broth is described as gram of product in solution per liter of fermentation broth (g/L) or as g/kg broth.

As used herein, the term “flux” is the rate of flow of molecules through a metabolic pathway, akin to the flow of material in a process.

“Bacteria”, or “eubacteria”, refers to a domain of prokaryotic organisms. Bacteria include gram-positive (gram+) bacteria and gram-negative (gram-) bacteria.

“Yeast” refers to a domain of eukaryotic organisms that are unicellular fungi.

The terms “derivative” and “analog” refer to a polypeptide differing from the enzymes of the invention, but retaining essential properties thereof. Generally, derivatives and analogs are overall closely similar, and, in many regions, identical to the enzymes of the invention. The terms “derived from”, “derivative” and “analog” when referring to enzymes of the invention include any polypeptides which retain at least some of the activity of the corresponding native polypeptide or the activity of its catalytic domain.

Derivatives of enzymes disclosed herein are polypeptides which may have been altered so as to exhibit features not found on the native polypeptide. Derivatives can be covalently modified by substitution (e.g. amino acid substitution), chemical, enzymatic, or other appropriate means with a moiety other than a naturally occurring amino acid (e.g., a detectable moiety such as an enzyme or radioisotope). Examples of derivatives include fusion proteins, or proteins which are based on a naturally occurring protein sequence, but which have been altered. For example, proteins can be designed by knowledge of a particular amino acid sequence, and/or a particular secondary, tertiary, and quaternary structure. Derivatives include proteins that are modified based on the knowledge of a previous sequence, natural or synthetic, which is then optionally modified, often, but not necessarily to confer some improved function. These sequences, or proteins, are then said to be derived from a particular protein or amino acid sequence. In some embodiments of the invention, a derivative must retain at least about 50% identity, at least about 60% identity, at least about 70% identity, at least about 80% identity, at least about 90% identity, at least about 95% identity, at least about 97% identity, or at least about 99% identity to the sequence the derivative is “derived from.” In some embodiments of the invention, an enzyme is said to be derived from an enzyme naturally found in a particular species if, using molecular genetic techniques, the DNA sequence for part or all of the enzyme is amplified and placed into a new host cell.

“Isolated” from, as used herein, refers to a process whereby, using molecular biology techniques, genetic material is harvested from a particular organism often with the end goal of putting the general material into a non-native environment.

The term “percent identity”, as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences.

As known in the art, “similarity” between two polypeptides is determined by comparing the amino acid sequence and conserved amino acid substitutes thereto of the polypeptide to the sequence of a second polypeptide.

“Identity” and “similarity” can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5.

Suitable nucleic acid sequences or fragments thereof (isolated polynucleotides of the present invention) encode polypeptides that are at least about 70% to 75% identical to the amino acid sequences disclosed herein, at least about 80%, at least about 85%, or at least about 90% identical to the amino acid sequences disclosed herein, or at least about 95%, at least about 96%, at least about 97%, at least about 98%, at least about 99%, or at least about 100% identical to the amino acid sequences disclosed herein. Suitable nucleic acid fragments are at least about 70%, at least about 75%, or at least about 80% identical to the nucleic acid sequences disclosed herein, at least about 80%, at least about 85%, or at least about 90% identical to the nucleic acid sequences disclosed herein, or at least about 95%, at least about 96%, at least about 97%, at least about 98%, at least about 99%, or at least about 100% identical to the nucleic acid sequences disclosed herein. Suitable nucleic acid fragments not only have the above identities/similarities but typically encode a polypeptide having at least about 50 amino acids, at least about 100 amino acids, at least about 150 amino acids, at least about 200 amino acids, or at least about 250 amino acids.

### Codon Optimization

In some embodiments of the present invention, exogenous genes may be codon-optimized in order to express the polypeptide they encode most efficiently in the host cell. Methods of codon optimization are well known in the art. (See, e.g. Welch et al. “Designing genes for successful protein expression.” Methods Enzymol. 2011. 498:43-66.)

In general, highly expressed genes in an organism are biased towards codons that are recognized by the most abundant tRNA species in that organism. One measure of this bias is the “codon adaptation index” or “CAI,” which measures the extent to which the codons used to encode each amino acid in a particular gene are those which occur most frequently in a reference set of highly expressed genes from an organism. The Codon Adaptation Index is described in more detail in Sharp et al., “The Codon Adaptation Index: a Measure of Directional Synonymous Codon Usage Bias, and Its Potential Applications.” Nucleic Acids Research (1987) 15: 1281-1295, which is incorporated by reference herein in its entirety.

A codon optimized sequence may be further modified for expression in a particular organism, depending on that organism's biological constraints. For example, large runs of “As” or “Ts” (e.g., runs greater than 3, 4, 5, 6, 7, 8, 9, or 10 consecutive bases) can effect transcription negatively. Therefore, it can be useful to remove a run by, for example, replacing at least one nucleotide in the run with another nucleotide. Furthermore, specific restriction enzyme sites may be removed for molecular cloning purposes by replacing at least one nucleotide in the restriction site with another nucleotide. Examples of such restriction enzyme sites include PacI, AscI, BamHI, BglII, EcoRI and XhoI. Additionally, the DNA sequence can be checked for direct repeats, inverted repeats and mirror repeats with lengths of about 5, 6, 7, 8, 9 or 10 bases or longer. Runs of “As” or “Ts”, restriction sites and/or repeats can be modified by replacing at least one codon within the sequence with the “second best” codons, i.e., the codon that occurs at the second highest frequency for a particular amino acid within the particular organism for which the sequence is being optimized.

Deviations in the nucleotide sequence that comprise the codons encoding the amino acids of any polypeptide chain allow for variations in the sequence coding for the gene. Since each codon consists of three nucleotides, and the nucleotides comprising DNA are restricted to four specific bases, there are 64 possible combinations of nucleotides, 61 of which encode amino acids (the remaining three codons encode signals ending translation). The “genetic code” which shows which codons encode which amino acids is reproduced herein as Table 1. As a result, many amino acids are designated by more than one codon. For example, the amino acids alanine and proline are coded for by four triplets, serine and arginine by six triplets each, whereas tryptophan and methionine are coded for by just one triplet. This degeneracy allows for DNA base composition to vary over a wide range without altering the amino acid sequence of the proteins encoded by the DNA.

TABLE 1
The Standard Genetic Code

| | T | C | A | G |
|---|---|---|---|---|
| T | TTT Phe (F) | TCT Ser (S) | TAT Tyr (Y) | TGT Cys (C) |
| | TTC ″ | TCC ″ | TAC ″ | TGC |
| | TTA Leu (L) | TCA ″ | TAA Ter | TGA Ter |
| | TTG ″ | TCG ″ | TAG Ter | TGG Trp (W) |
| C | CTT Leu (L) | CCT Pro (P) | CAT His (H) | CGT Arg (R) |
| | CTC ″ | CCC ″ | CAC ″ | CGC ″ |
| | CTA ″ | CCA ″ | CAA Gln (Q) | CGA ″ |
| | CTG ″ | CCG ″ | CAG ″ | CGG ″ |
| A | ATT Ile (I) | ACT Thr (T) | AAT Asn (N) | AGT Ser (S) |
| | ATC ″ | ACC ″ | AAC ″ | AGC ″ |
| | ATA ″ | ACA ″ | AAA Lys (K) | AGA Arg (R) |
| | ATG Met | ACG ″ | AAG ″ | AGG ″ |
| | (M) | | | |
| G | GTT Val (V) | GCT Ala (A) | GAT Asp (D) | GGT Gly (G) |
| | GTC ″ | GCC ″ | GAC ″ | GCC ″ |
| | GTA ″ | GCA ″ | GAA Glu (E) | GGA ″ |
| | GTG ″ | GCG ″ | GAG ″ | GGG ″ |

Many organisms display a bias for use of particular codons to code for insertion of a particular amino acid in a growing peptide chain. Codon preference or codon bias, differences in codon usage between organisms, is afforded by degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

### Host Cells

In some embodiments of the invention, the host cell is a eukaryotic microorganism. In some embodiments, the host cell is a yeast. In some embodiments, the host cell is able to digest and ferment cellulose. In some embodiments, the host cell is from the genus Saccharomyces. In some embodiments, the host cell is Saccharomyces cerevisiae.

In some embodiments, the host cells of the invention are cultured at a temperature above about 20° C., above about 25° C., above about 27° C., above about 30° C., above about 33° C., above about 35° C., above about 37° C., above about 40° C., above about 43° C., above about 45° C., or above about 47° C. In some embodiments, the host cells of the invention contain genetic constructs that lead to the down-regulation of one or more genes encoding a polypeptide at least about 80%, at least about 85%, at least about 90%, at least about 95%, at least about 96%, at least about 97%, at least about 98%, at least about 99%, or about 100% identical to one or more of the polypeptides encoded SEQ ID NOs: 2, 5, 7, 25, 31, 55, 57, 159 and 161, and the polynucleotide sequence encoded by SEQ ID NO: 3. In some embodiments, the host cells of the invention contain genetic constructs that lead to the expression or up-regulation of a polypeptide encoding the activity associated with EC Nos.: 1.1.1.8, 3.1.3.21, 1.2.1.43, 1.2.1.2, 1.4.1.2, and 1.4.1.4.

In some embodiments, the host cells of the invention contain genetic constructs that lead to the expression or up-regulation of one or more genes encoding a polypeptide at least about 80%, at least about 85%, at least about 90%, at least about 95%, at least about 96%, at least about 97%, at least about 98%, at least about 99%, or about 100% identical to one or more of the polypeptides encoded by SEQ ID NOs: 9, 11, 13, 15, 17, 19, 21, 23, 27, 33, 35, 37, 39, 41, 43, 45, 47, 49, 51, 53, 157, and 163. In some embodiments, the host cells of the invention contain genetic constructs that lead to the expression or up-regulation of a polypeptide encoding the activity associated with EC Nos.: 1.1.1.1, 1.1.1.2, 1.2.1.3, 1.2.1.4, 1.2.1.10, 2.3.1.54, 1.97.1.4, 1.4.1.2, 1.4.1.4, 1.1.1.14, 1.4.1.13, 6.3.1.2, 6.3.4.6, and 3.2.1.3.

In some embodiments, bifunctional acetaldehyde-alcohol dehydrogenase is up-regulated. In some embodiments, the up-regulated bifunctional acetaldehyde-alcohol dehydrogenase is from an enzyme that corresponds to an EC number selected from the group consisting of: EC 1.2.1.0 and 1.1.1.1. In some embodiments, the bifunctional acetaldehyde-alcohol dehydrogenase is a NADPH dependent bifunctional acetaldehyde-alcohol dehydrogenase selected from a group of enzymes having the following Enzyme Commission Numbers: EC 1.2.1.10 and 1.1.1.2. In some embodiments, the bifunctional acetaldehyde-alcohol dehydrogenase corresponds to a polypeptide selected from the group consisting of SEQ ID NOs: 13, 15, and 17. In some embodiments, the bifunctional acetaldehyde-alcohol dehydrogenase is adhE.

In some embodiments, pyruvate formate lyase is up-regulated. In some embodiments, the up-regulated pyruvate formate lyase is from an enzyme that corresponds to EC 2.3.1.54. In some embodiments, the pyruvate formate lyase corresponds to a polypeptide encoded by SEQ ID NO: 2. In some embodiments, pyruvate formate lyase activating enzyme is up-regulated. In some embodiments, the up-regulated pyruvate formate lyase activating enzyme is from an enzyme that corresponds to EC 1.97.1.4. In some embodiments, the pyruvate formate lyase activating enzyme corresponds to a polynucleotide encoded by SEQ ID NO: 3.

In some embodiments, glutamate dehydrogenase is up-regulated. In some embodiments, the glutamate dehydrogenase that is up-regulated is NADH-dependent. In some embodiments, the up-regulated glutamate dehydrogenase corresponds to EC 1.4.1.2. In some embodiments, glutamate dehydrogenase from S. cerevisiae is up-regulated. In some embodiments, the glutamate dehydrogenase that is up-regulated is from S. cerevisiae is GDH2 and corresponds to a polypeptide corresponding to SEQ ID NO: 29. In some embodiments, glutamate synthase is up-regulated. In some embodiments, the up-regulated glutamate synthase corresponds to EC 1.4.1.14. In some embodiments, glutamate synthase from S. cerevisiae is up-regulated. In some embodiments, the glutamate dehydrogenase that is up-regulated is from S. cerevisiae is GLT1 and corresponds to a polypeptide corresponding to SEQ ID NO: 33. In some embodiments, glutamine synthase is up-regulated. In some embodiments, the up-regulated glutamine synthase corresponds to EC 6.3.1.2. In some embodiments, glutamine synthase from S. cerevisiae is up-regulated. In some embodiments, the glutamine dehydrogenase that is up-regulated is from S. cerevisiae is GLN1 and corresponds to a polypeptide corresponding to SEQ ID NO: 35.

In some embodiments, a urea-amido lyase is up-regulated. In some embodiments, the up-regulated urea-amido lyase corresponds to EC 6.3.4.6. In some embodiments, urea-amido lyase from S. cerevisiae is up-regulated. In some embodiments, the urea-amido lyase that is up-regulated is from S. cerevisiae is DUR1/2 and corresponds to a polypeptide corresponding to SEQ ID NO: 37.

In some embodiments, a protease is up-regulated. In some embodiments, the up-regulated protease corresponds to EC 3.4.23.41. In some embodiments, the protease is an endoprotease. In some embodiments, the protease is an exoprotease. In some embodiments, a protease from Z. mays, N. crassa, P. anserine, or M. oryzae is up-regulated. In some embodiments, the protease that is up-regulated corresponds to a polypeptide corresponding to SEQ ID NOs: 41, 43, 45, 47, 49 or 51. In some embodiments, a permease is up-regulated. In some embodiments, a permease from S. cerevisiae is up-regulated. In some embodiments, the permease that is up-regulated is GAP1 and corresponds to a polypeptide corresponding to SEQ ID NO: 53.

In some embodiments, a glucoamylase is up-regulated. In some embodiments, the up-regulated glucoamylase corresponds to EC 3.2.1.3. In some embodiments, a glucoamylase from S. fibuligera is up-regulated. In some embodiments, the glucoamylase from S. fibuligera that is up-regulated corresponds to a polypeptide corresponding to SEQ ID NO: 163.

In some embodiments, an ammonium transporter is up-regulated. In some embodiments, an ammonium transporter from S. cerevisiae is up-regulated. In some embodiments, the ammonium transporter that is up-regulated is MEP1. MEP2, or MEP3 from S. cerevisiae and corresponds to a polypeptide corresponding to SEQ ID NOs: 19, 21, and 23. In some embodiments, a urea transporter is up-regulated. In some embodiments, a urea transporter from is from S. cerevisiae. In some embodiments, the urea transporter that is up-regulated is DUR3 or DUR4 from S. cerevisiae and corresponds to a polypeptide corresponding to SEQ ID NOs: 39.

In some embodiments, glycerol-3-phosphate dehydrogenase is down-regulated. In some embodiments, the down-regulated Gpd is from an enzyme that corresponds to EC 1.1.1.8. In some embodiments, the glycerol-3-phosphate dehydrogenase is selected from the group consisting of glycerol-3-phosphate dehydrogenase 1 (“Gpd1”), glycerol-3-phosphate dehydrogenase 2 (“Gpd2”), and combinations thereof. In some embodiments, the Gpd1 is from S. cerevisiae and corresponds to a polypeptide encoded by SEQ ID NO: 5. In some embodiments, the Gpd2 is from S. cerevisiae and corresponds to a polypeptide encoded by SEQ ID NO: 7. In some embodiments, formate dehydrogenase is down-regulated. In some embodiments, the down-regulated formate dehydrogenase corresponds to an EC number selected from the group consisting of: EC 1.2.1.43 and EC 1.2.1.2. In some embodiments, formate dehydrogenase from S. cerevisiae is down-regulated. In some embodiments, the formate dehydrogenase from S. cerevisiae corresponds to a polypeptide corresponding to SEQ ID NO: 2 or a polynucleotide corresponding to SEQ ID NO: 3. In some embodiments, glycerol-3-phosphate phosphatase is down-regulated. In some embodiments, the down-regulated glycerol-3-phosphate phosphatase corresponds to EC 3.1.3.21. In some embodiments, the down-regulated glycerol-3-phosphate phosphatase corresponds to a polynucleotide corresponding to SEQ ID NOs 158 or 160 or a polypeptide corresponding to SEQ ID NOs 159 or 161.

In some embodiments, glutamate dehydrogenase is down-regulated. In some embodiments, the glutamate dehydrogenase that is down-regulated is NADPH-dependent. In some embodiments, the down-regulated glutamate dehydrogenase corresponds to EC 1.4.1.4. In some embodiments, glutamate dehydrogenase that is down-regulated is from S. cerevisiae. In some embodiments, the glutamate dehydrogenase is from S. cerevisiae is GDH1 and corresponds to a polypeptide corresponding to SEQ ID NO: 25.

In some embodiments, a regulatory element is down-regulated. In some embodiments, the regulatory element that is down-regulated is from S. cerevisiae. In some embodiments, the regulatory element from S. cerevisiae is Ure2 and corresponds to a polypeptide corresponding to SEQ ID NO: 55. In some embodiments, the regulatory element from S. cerevisiae is Aua1 and corresponds to a polypeptide corresponding to SEQ ID NO: 57.

In some embodiments, bifunctional acetaldehyde-alcohol dehydrogenase (AdhE), B. adolescentis pyruvate formate lyase, and B adolescentis pyruvate formate lyase activating enzyme are up-regulated, and Gpd1 and Gpd2 are down-regulated. In some embodiments, AdhE, B. adolescentis pyruvate formate lyase, and B adolescentis pyruvate formate lyase activating enzyme are up-regulated, and Gpd1, Gpd2, Fdh1 and Fdh2 are down-regulated. In some embodiments, AdhE, B. adolescentis pyruvate formate lyase, and B adolescentis pyruvate formate lyase activating enzyme are up-regulated, Gpd1, Gpd2, Fdh1 and Fdh2 are down-regulated, GPD1 is expressed under the control of the GPD2 promoter, and GPD2 is expressed under the control of the GPD1 promoter. In some embodiments, AdhE, B. adolescentis pyruvate formate lyase, and B adolescentis pyruvate formate lyase activating enzyme are up-regulated, Gpd1, Gpd2, Fdh1, Fdh2, Gdh1 are down-regulated, GPD1 is expressed under the control of the GPD2 promoter, and GPD2 is expressed under the control of the GPD1 promoter. In some embodiments, AdhE, B. adolescentis pyruvate formate lyase, and B adolescentis pyruvate formate lyase activating enzyme, and Glt1 are up-regulated, Gpd1, Gpd2, Fdh1, Fdh2, Gdh1 are down-regulated, GPD1 is expressed under the control of the GPD2 promoter, and GPD2 is expressed under the control of the GPD1 promoter. In some embodiments, AdhE, B. adolescentis pyruvate formate lyase, and B adolescentis pyruvate formate lyase activating enzyme, and Gln1 are up-regulated, Gpd1, Gpd2, Fdh1, Fdh2, Gdh1 are down-regulated, GPD1 is expressed under the control of the GPD2 promoter, and GPD2 is expressed under the control of the GPD1 promoter. In some embodiments, AdhE, B. adolescentis pyruvate formate lyase, and B adolescentis pyruvate formate lyase activating enzyme, Gln1 and Glt1 are up-regulated, Gpd1, Gpd2, Fdh1, Fdh2, Gdh1 are down-regulated, GPD1 is expressed under the control of the GPD2 promoter, and GPD2 is expressed under the control of the GPD1 promoter. In some embodiments, the regulatory element Ure2 is down-regulated. In some embodiments, the regulatory element Aua1 is down-regulated. In some embodiments, Gln3 is up-regulated.

In some embodiments, AdhE, B. adolescentis pyruvate formate lyase, and B adolescentis pyruvate formate lyase activating enzyme are up-regulated, and Gpd2, Fdh1, and Fdh2 are down-regulated. In some embodiments, AdhE, B. adolescentis pyruvate formate lyase, and B adolescentis pyruvate formate lyase activating enzyme are up-regulated, and Gpd2, Fdh1, Fdh2, and Gdh1 are down-regulated. In some embodiments, AdhE, B. adolescentis pyruvate formate lyase, and B adolescentis pyruvate formate lyase activating enzyme are up-regulated, and Gpd1, Fdh1, and Fdh2 are down-regulated. In some embodiments, AdhE, B. adolescentis pyruvate formate lyase, and B adolescentis pyruvate formate lyase activating enzyme are up-regulated, and Gpd1, Fdh1, Fdh2, and Gdh1 are down-regulated. In some embodiments, Dur1/2 is additionally expressed. In some embodiments, Dur1/2 is expressed from the TEF2 promoter. In some embodiments, Dur1/2 is expressed from the HXT7 promoter. In some embodiments, Dur1/2 is expressed from the GPM1 promoter. In some embodiments, Dur1/2 is expressed from the ADH1 promoter. In some embodiments, Dur1/2 is expressed from the HXT7 TEF2 promoters. In some embodiments, Gln3 is up-regulated. In some embodiments, GPD1 is expressed from the GPD2 promoter. In some embodiments, GPD2 is expressed from a GPD1 promoter. Ethanol Production

For a microorganism to produce ethanol most economically, it is desired to produce a high yield. In one embodiment, the only product produced is ethanol. Extra products lead to a reduction in product yield and an increase in capital and operating costs, particularly if the extra products have little or no value. Extra products also require additional capital and operating costs to separate these products from ethanol.

Ethanol production can be measured using any method known in the art. For example, the quantity of ethanol in fermentation samples can be assessed using HPLC analysis. Additionally, many ethanol assay kits are commercially available, for example, alcohol oxidase enzyme based assays. Methods of determining ethanol production are within the scope of those skilled in the art from the teachings herein.

In some embodiments of the invention where redirected carbon flux generates increased ethanol production, the ethanol output can be improved by growth-coupled selection. For example, continuous culture or serial dilution cultures can be performed to select for cells that grow faster and/or produce ethanol (or any desired product) more efficiently on a desired feedstock.

One embodiment of the present invention relates to a method of producing ethanol using a microorganism described herein wherein the microorganism is cultured in the presence of a carbon containing feedstock for sufficient time to produce ethanol and, optionally, extracting the ethanol. In some embodiments, nitrogen is added to the culture containing the recombinant microorganism and the feedstock.

Ethanol may be extracted by methods known in the art. (See, e.g., U.S. Appl. Pub. No. 2011/0171709, which is incorporated herein by reference in its entirety.)

Another embodiment of the present invention relates to a method of producing ethanol using a co-culture composed of at least two microorganisms in which at least one of the organisms is an organism described herein, and at least one of the organisms is a genetically distinct microorganism. In some embodiments, the genetically distinct microorganism is a yeast or bacterium. In some embodiments the genetically distinct microorganism is any organism from the genus Issatchenkia, Pichia, Clavispora, Candida, Hansenula, Kluyveromyces, Saccharomyces, Trichoderma, Thermoascus, Escherichia, Clostridium, Caldicellulosiruptor, Thermoanaerobacter and Thermoanaerobacterium.

In some embodiments, the recombinant microorganism produces about 2% to about 3% higher ethanol titer than a wildtype, non-recombinant organism; at least about 1% to at least about 2% higher ethanol titer than a wildtype, non-recombinant organism; at least about 1% to at least about 5% higher ethanol titer than a wildtype, non-recombinant organism; at least about 1% to at least about 7% higher ethanol titer than a wildtype, non-recombinant organism; at least about 1% to at least about 10% higher ethanol titer than a wildtype, non-recombinant organism; at least about 1% to at least about 15% higher ethanol titer than a wildtype, non-recombinant organism; at least about 1% to at least about 20% higher ethanol titer than a wildtype, non-recombinant organism; at least about 1% to at least about 30% higher ethanol titer than a wildtype, non-recombinant organism; at least about 1% to at least about 50% higher ethanol titer than a wildtype, non-recombinant organism; at least about 1% to at least about 75% higher ethanol titer than a wildtype, non-recombinant organism; at least about 1% to at least about 100% higher ethanol titer than a wildtype, non-recombinant organism. In some embodiments, the recombinant microorganism produces at least about 0.5 g/L ethanol to at least about 2 g/L ethanol, at least about 0.5 g/L ethanol to at least about 3 g/L ethanol, at least about 0.5 g/L ethanol to at least about 5 g/L ethanol, at least about 0.5 g/L ethanol to at least about 7 g/L ethanol, at least about 0.5 g/L ethanol to at least about 10 g/L ethanol, at least about 0.5 g/L ethanol to at least about 15 g/L ethanol, at least about 0.5 g/L ethanol to at least about 20 g/L ethanol, at least about 0.5 g/L ethanol to at least about 30 g/L ethanol, at least about 0.5 g/L ethanol to at least about 40 g/L ethanol, at least about 0.5 g/L ethanol to at least about 50 g/L ethanol, at least about 0.5 g/L ethanol to at least about 75 g/L ethanol, at least about 0.5 g/L ethanol to at least about 99 g/L ethanol, at least about 0.5 g/L ethanol to at least about 125 g/L ethanol, or at least about 0.5 g/L to at least about 150 g/L ethanol per at least about 24 hour, at least about 48 hour, or at least about 72 hour incubation on a carbon-containing feed stock, such as corn mash.

In some embodiments, the recombinant microorganism produces ethanol at least about 55% to at least about 75% of theoretical yield, at least about 50% to at least about 80% of theoretical yield, at least about 45% to at least about 85% of theoretical yield, at least about 40% to at least about 90% of theoretical yield, at least about 35% to at least about 95% of theoretical yield, at least about 30% to at least about 99% of theoretical yield, or at least about 25% to at least about 99% of theoretical yield. In some embodiments, methods of producing ethanol can comprise contacting a biomass feedstock with a host cell or co-culture of the invention and additionally contacting the biomass feedstock with externally produced saccharolytic enzymes. In some embodiments, the host cells are genetically engineered (e.g., transduced, transformed, or transfected) with the polynucleotides encoding saccharolytic enzymes.

An “amylolytic enzyme” can be any enzyme involved in amylase digestion, metabolism and/or hydrolysis. The term “amylase” refers to an enzyme that breaks starch down into sugar. Amylase is present in human saliva, where it begins the chemical process of digestion. Foods that contain much starch but little sugar, such as rice and potato, taste slightly sweet as they are chewed because amylase turns some of their starch into sugar in the mouth. The pancreas also makes amylase (α-amylase) to hydrolyse dietary starch into disaccharides and trisaccharides which are converted by other enzymes to glucose to supply the body with energy. Plants and some bacteria also produce amylase. All amylases are glycoside hydrolases and act on α-1,4-glycosidic bonds. Some amylases, such as γ-amylase (glucoamylase), also act on α-1,6-glycosidic bonds. Amylase enzymes include α-amylase (EC 3.2.1.1), β-amylase (EC 3.2.1.2), and 7-amylase (EC 3.2.1.3). The α-amylases are calcium metalloenzymes, unable to function in the absence of calcium. By acting at random locations along the starch chain, α-amylase breaks down long-chain carbohydrates, ultimately yielding maltotriose and maltose from amylose, or maltose, glucose and “limit dextrin” from amylopectin. Because it can act anywhere on the substrate, α-amylase tends to be faster-acting than β-amylase. In animals, it is a major digestive enzyme and its optimum pH is about 6.7-7.0. Another form of amylase, β-amylase is also synthesized by bacteria, fungi, and plants. Working from the non-reducing end, β-amylase catalyzes the hydrolysis of the second α-1,4 glycosidic bond, cleaving off two glucose units (maltose) at a time. Many microbes produce amylase to degrade extracellular starches. In addition to cleaving the last α(1-4) glycosidic linkages at the nonreducing end of amylose and amylopectin, yielding glucose, 7-amylase will cleave α(1-6) glycosidic linkages. Another amylolytic enzyme is alpha-glucosidase that acts on maltose and other short malto-oligosaccharides produced by alpha-, beta-, and gamma-amylases, converting them to glucose. Another amylolytic enzyme is pullulanase. Pullulanase is a specific kind of glucanase, an amylolytic exoenzyme, that degrades pullulan. Pullulan is regarded as a chain of maltotriose units linked by alpha-1,6-glycosidic bonds. Pullulanase (EC 3.2.1.41) is also known as pullulan-6-glucanohydrolase (debranching enzyme). Another amylolytic enzyme, isopullulanase, hydrolyses pullulan to isopanose (6-alpha-maltosylglucose). Isopullulanase (EC 3.2.1.57) is also known as pullulan 4-glucanohydrolase. An “amylase” can be any enzyme involved in amylase digestion, metabolism and/or hydrolysis, including α-amylase, β-amylase, glucoamylase, pullulanase, isopullulanase, and alpha-glucosidase.

In some embodiments, the recombinant microorganisms of the invention further comprise one or more native and/or heterologous enzymes which encodes a saccharolytic enzyme, including amylases, cellulases, hemicellulases, cellulolytic and amylolytic accessory enzymes, inulinases, levanases, and pentose sugar utilizing enzymes. In one aspect, the saccharolytic enzyme is an amylase, where the amylase is selected from H. grisea, T. aurantiacus, T. emersonii, T. reesei, C. lacteus, C. formosanus, N. takasagoensis, C. acinaciformis, M. darwinensis, N. walkeri, S. fibuligera, C. luckowense R. speratus, Thermobfida fusca, Clostridum thermocellum, Clostridium cellulolyticum, Clostridum josui, Bacillus pumilis, Cellulomonas fimi, Saccharophagus degradans, Piromyces equii, Neocallimastix patricarum or Arabidopsis thaliana. In another aspect, the saccharolytic enzyme is a glucoamylase (glu-0111-CO) from S. fibuligera.

The term “xylanolytic activity” is intended to include the ability to hydrolyze glycosidic linkages in oligopentoses and polypentoses. The term “xylanase” is the name given to a class of enzymes which degrade the linear polysaccharide beta-1,4-xylan into xylose, thus breaking down hemicellulose, one of the major components of plant cell walls. As such, it plays a major role in micro-organisms thriving on plant sources (mammals, conversely, do not produce xylanase). Additionally, xylanases are present in fungi for the degradation of plant matter into usable nutrients. Xylanases include those enzymes that correspond to E.C. Number 3.2.1.8. A “xylose metabolizing enzyme” can be any enzyme involved in xylose digestion, metabolism and/or hydrolysis, including a xylose isomerase, xylulokinase, xylose reductase, xylose dehydrogenase, xylitol dehydrogenase, xylonate dehydratase, xylose transketolase, and a xylose transaldolase protein.

The term “pectinase” is a general term for enzymes, such as pectolyase, pectozyme and polygalacturonase, commonly referred to in brewing as pectic enzymes. These enzymes break down pectin, a polysaccharide substrate that is found in the cell walls of plants. One of the most studied and widely used commercial pectinases is polygalacturonase. Pectinases are commonly used in processes involving the degradation of plant materials, such as speeding up the extraction of fruit juice from fruit, including apples and sapota. Pectinases have also been used in wine production since the 1960s.

A “saccharolytic enzyme” can be any enzyme involved in carbohydrate digestion, metabolism and/or hydrolysis, including amylases, cellulases, hemicellulases, cellulolytic and amylolytic accessory enzymes, inulinases, levanases, and pentose sugar utilizing enzymes.

A “pentose sugar utilizing enzyme” can be any enzyme involved in pentose sugar digestion, metabolism and/or hydrolysis, including xylanase, arabinase, arabinoxylanase, arabinosidase, arabinofuranosidase, arabinoxylanase, arabinosidase, and arabinofuranosidase, arabinose isomerase, ribulose-5-phosphate 4-epimerase, xylose isomerase, xylulokinase, xylose reductase, xylose dehydrogenase, xylitol dehydrogenase, xylonate dehydratase, xylose transketolase, and/or xylose transaldolase.

### Glycerol Production

In some embodiments of the invention where redirected carbon flux generates increased ethanol production, the glycerol output can be decreased by growth-coupled selection. For example, continuous culture or serial dilution cultures can be performed to select for cells that produce less glycerol on a desired feedstock. Glycerol can be measured, for example, by HPLC analysis of metabolite concentrations.

In some embodiments, the recombinant microorganism produces at least about 20% to at least about 30% less glycerol than a wildtype, non-recombinant organism; at least about 30% to at least about 50% less glycerol than a wildtype, non-recombinant organism; at least about 40% to at least about 60% less glycerol than a wildtype, non-recombinant organism; at least about 50% to at least about 70% less glycerol than a wildtype, non-recombinant organism; at least about 60% to at least about 80% less glycerol than a wildtype, non-recombinant organism; at least about 70% to at least about 90% less glycerol than a wildtype, non-recombinant organism; at least about 75% to at least about 95% less glycerol than a wildtype, non-recombinant organism; at least about 70% to at least about 99% less glycerol than a wildtype, non-recombinant organism; at least about 15% to at least about 30% less glycerol than a wildtype, non-recombinant organism; at least about 10% to at least about 40% less glycerol than a wildtype, non-recombinant organism; at least about 10% to at least about 50% less glycerol than a wildtype, non-recombinant organism; at least about 10% to at least about 60% less glycerol than a wildtype, non-recombinant organism; at least about 10% to at least about 70% less glycerol than a wildtype, non-recombinant organism; at least about 10% to at least about 80% less glycerol than a wildtype, non-recombinant organism; at least about 10% to at least about 90% less glycerol than a wildtype, non-recombinant organism; at least about 10% to at least about 99% less glycerol than a wildtype, non-recombinant organism; at least about 10% to at least about 100% less glycerol than a wildtype, non-recombinant organism; at least about 5% to at least about 100% less glycerol than a wildtype, non-recombinant organism; at least about 1% to at least about 100% less glycerol than a wildtype, non-recombinant organism. In some embodiments, the recombinant microorganism produces no glycerol. In some embodiments, the recombinant microorganism has a growth rate at least about ½ to at least about equal to the growth rate of a wildtype, non-recombinant organism, at least about ¼ to at least about equal to the growth rate of a wildtype, non-recombinant organism, at least about ⅛ to at least about equal to the growth rate of a wildtype, non-recombinant organism, at least about 1/10 to at least about equal to the growth rate of a wildtype, non-recombinant organism, at least about 1/25 to at least about equal to the growth rate of a wildtype, non-recombinant organism, at least about 1/50 to at least about equal to the growth rate of a wildtype, non-recombinant organism or at least about 1/100th to at least about equal to the growth rate of a wildtype, non-recombinant organism.

A wildtype-non-recombinant organism produces glycerol at a rate of at least about 8-11 mM glycerol per gram dry cell weight (DCW) during anaerobic growth. In some embodiments, glycerol production is reduced to a rate of between 1-10 mM glycerol per gram dry cell weight during anaerobic growth.

### EXAMPLES

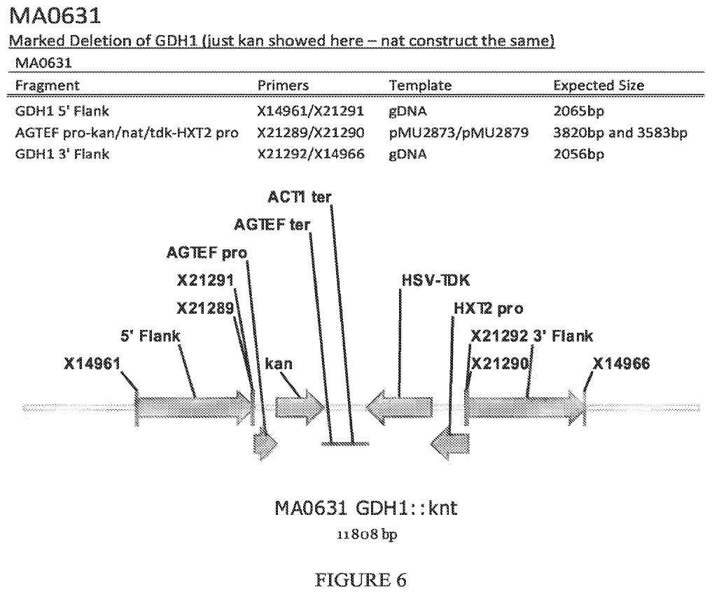

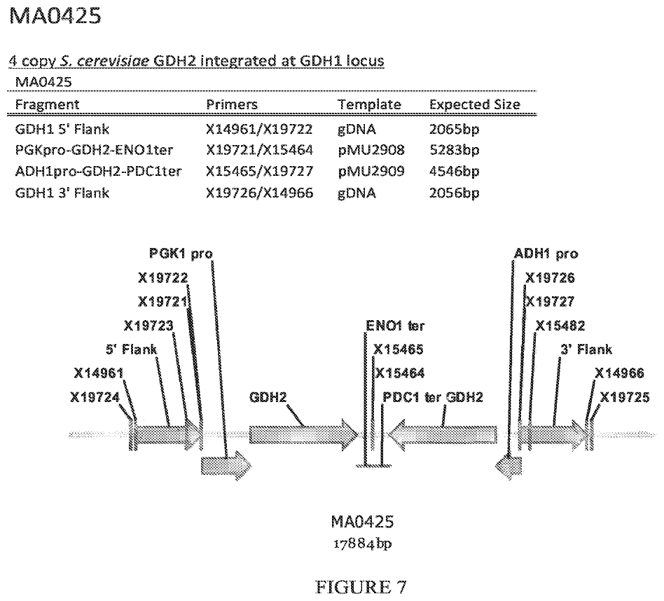

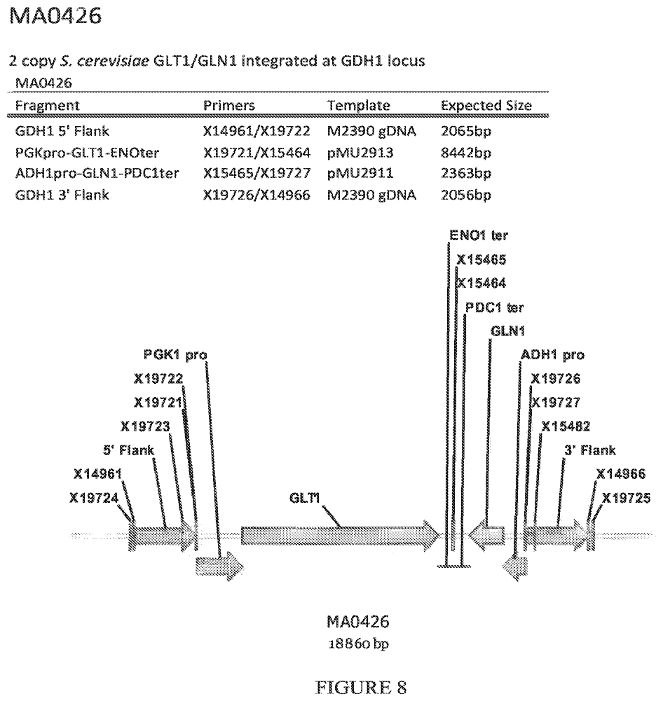

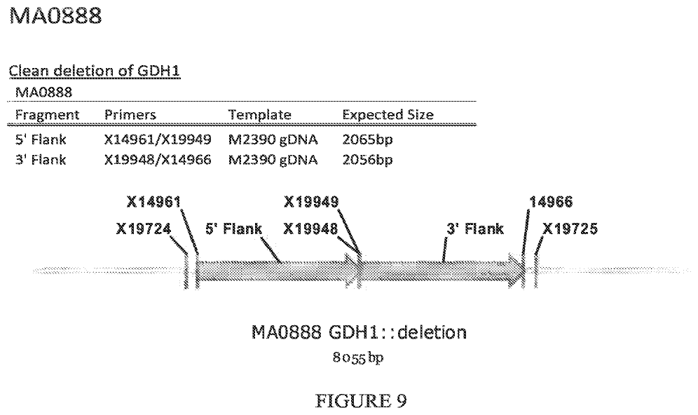

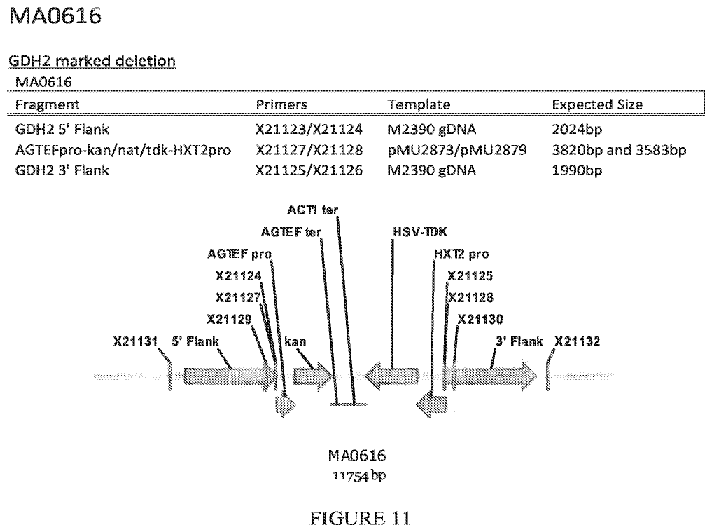

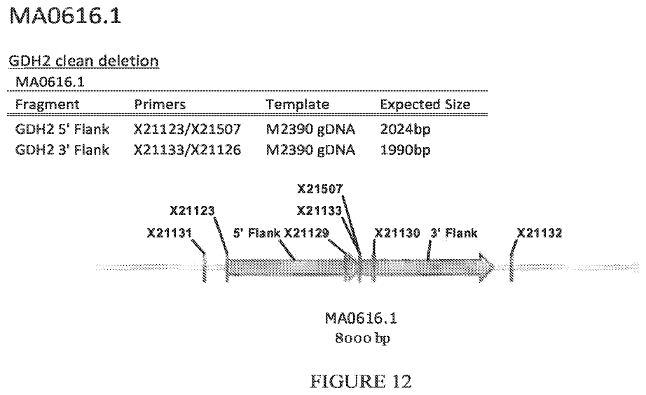

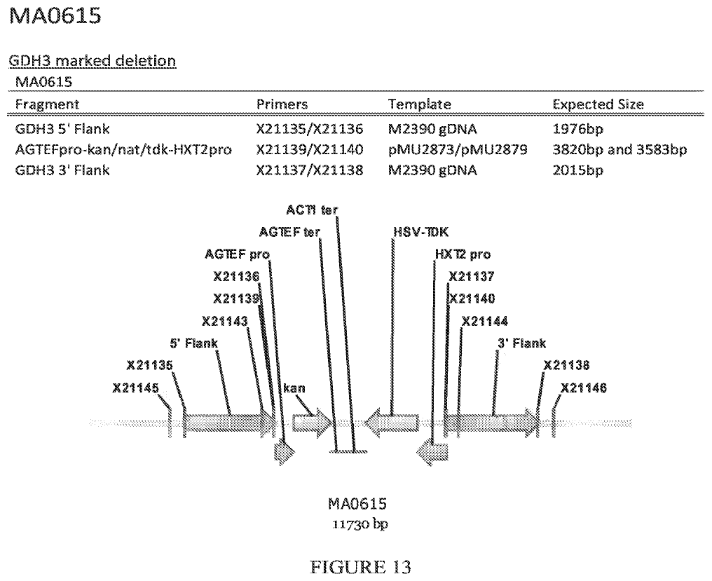

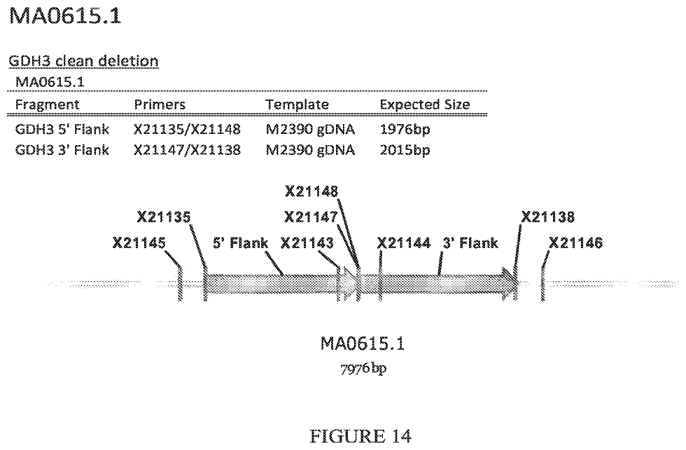

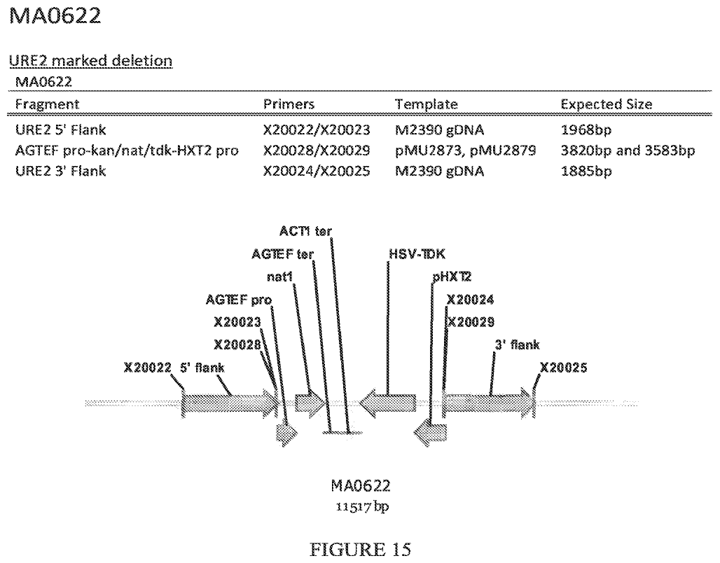

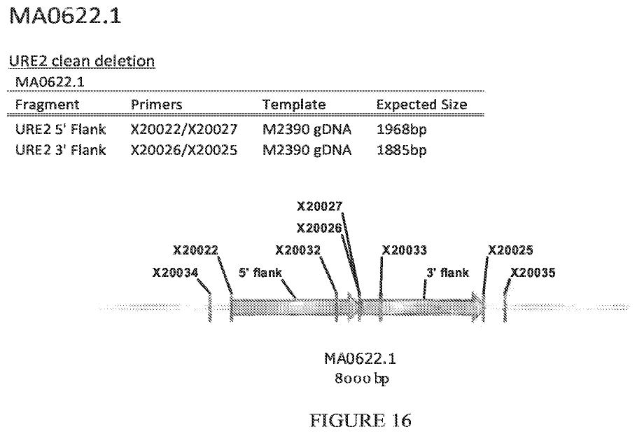

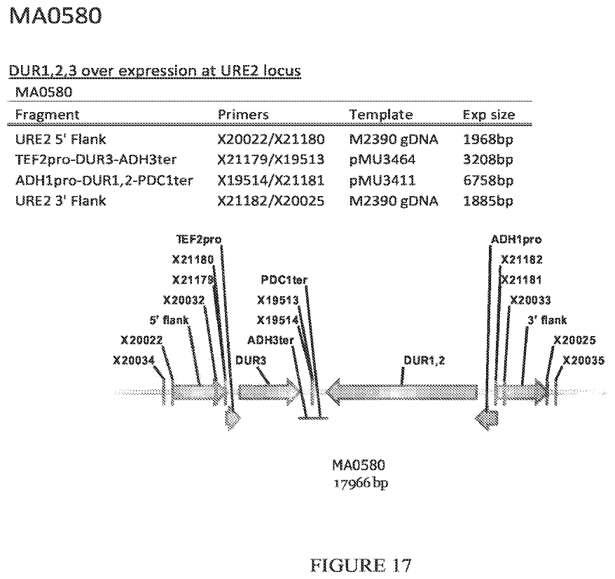

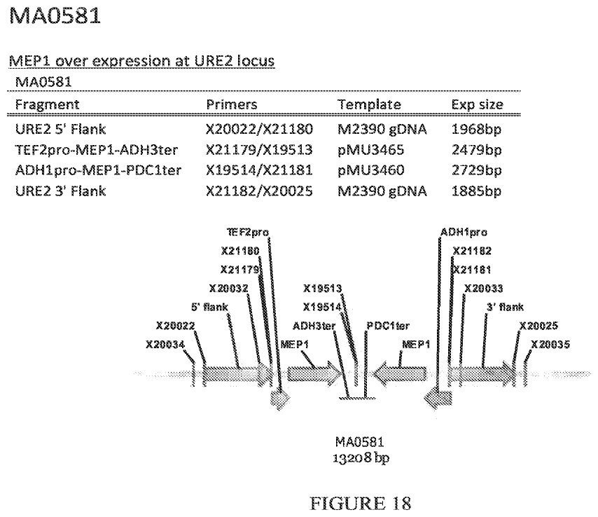

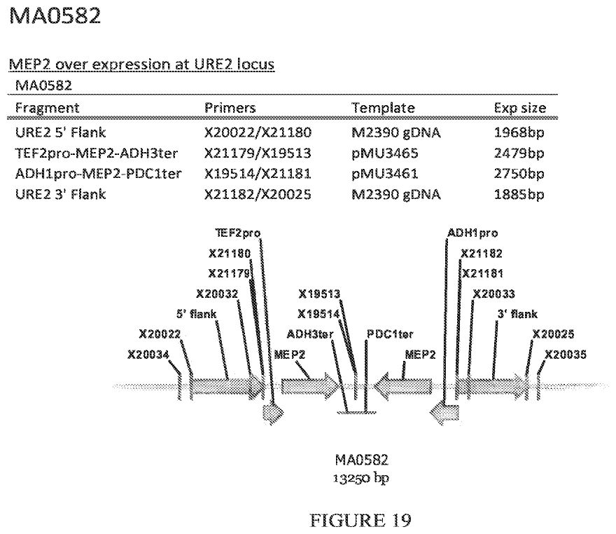

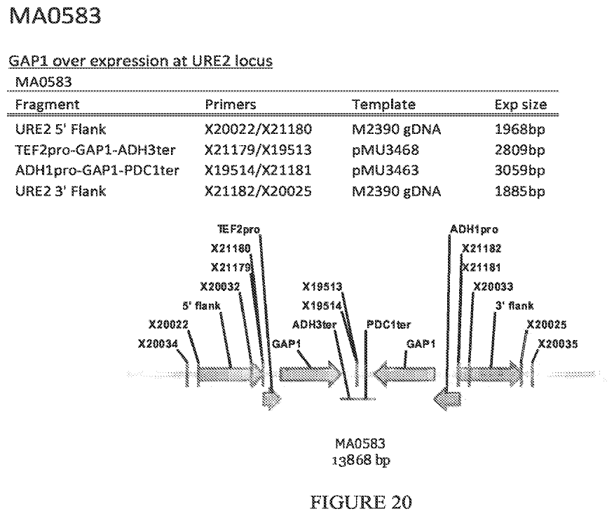

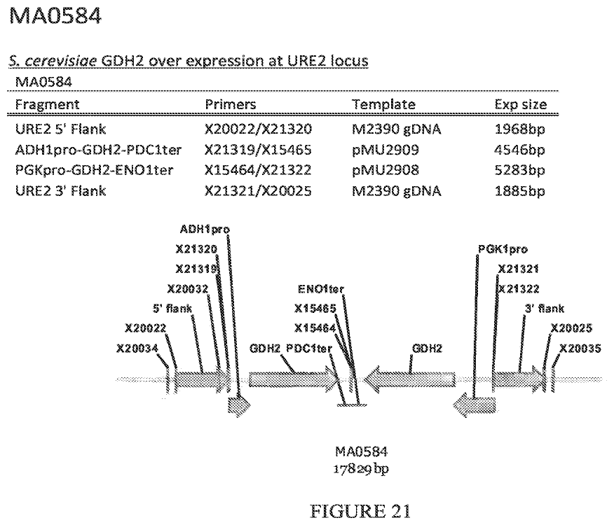

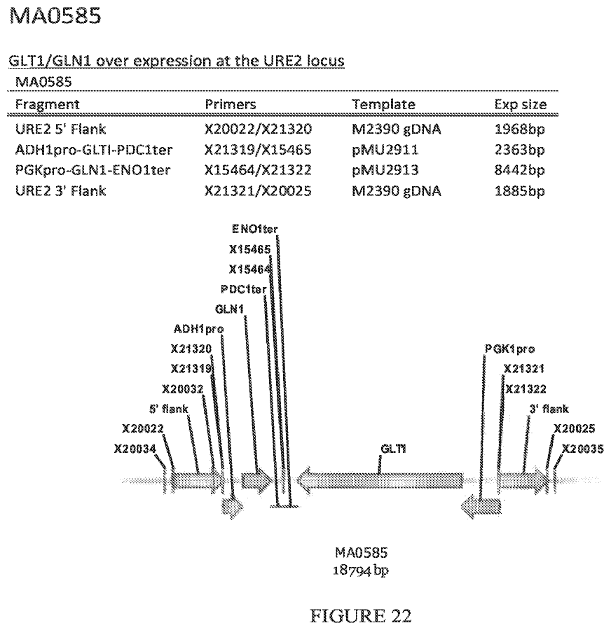

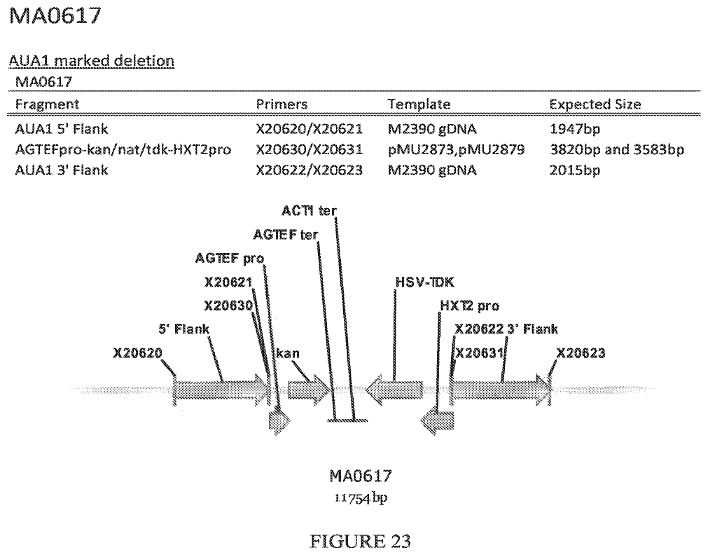

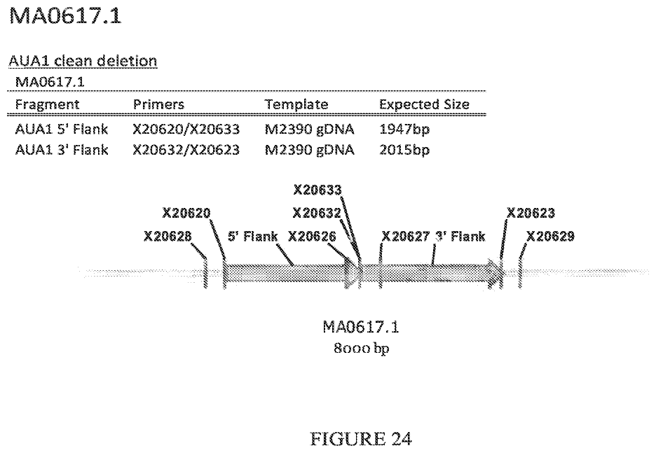

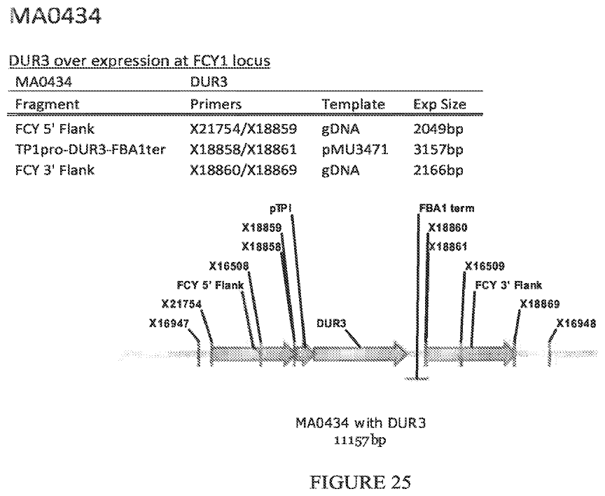

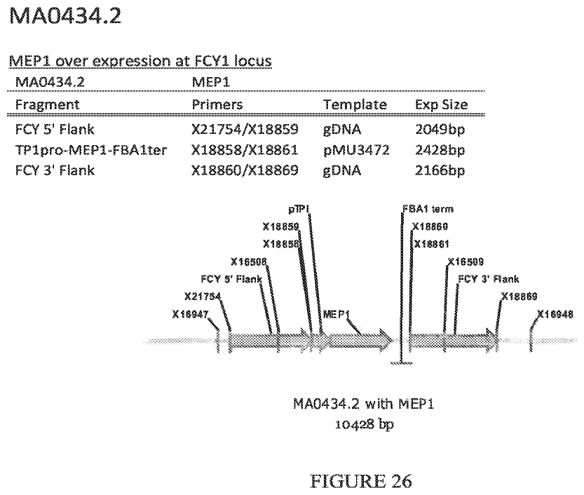

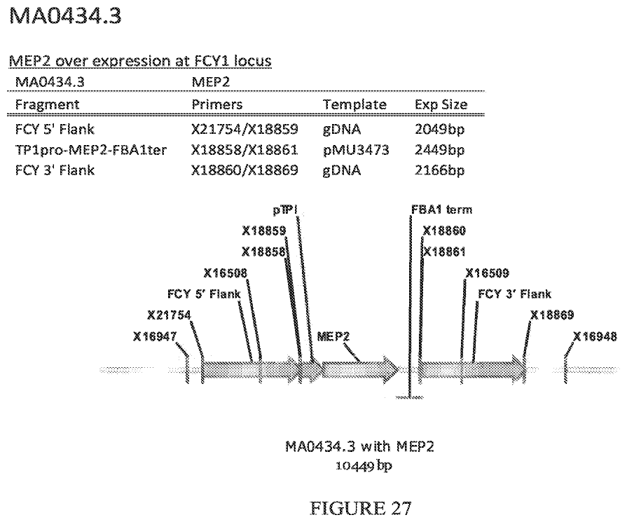

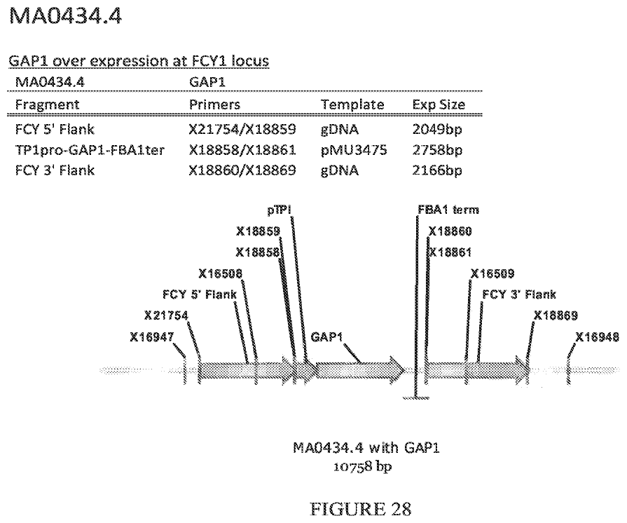

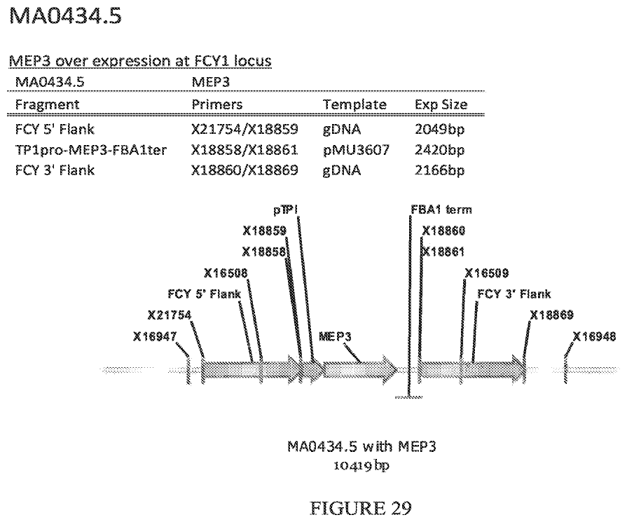

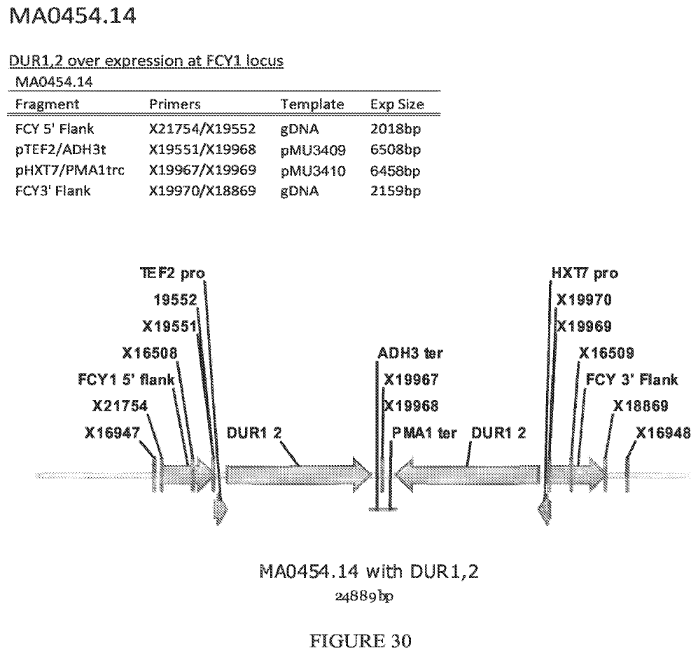

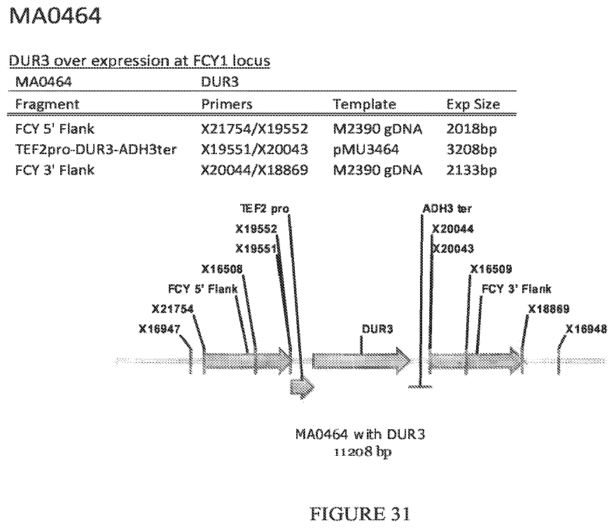

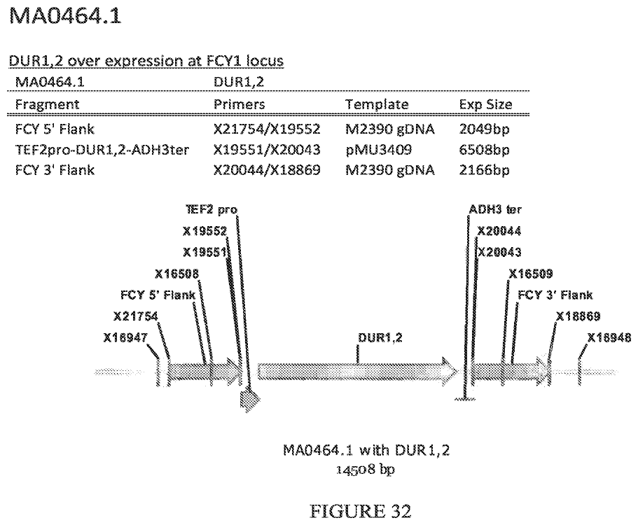

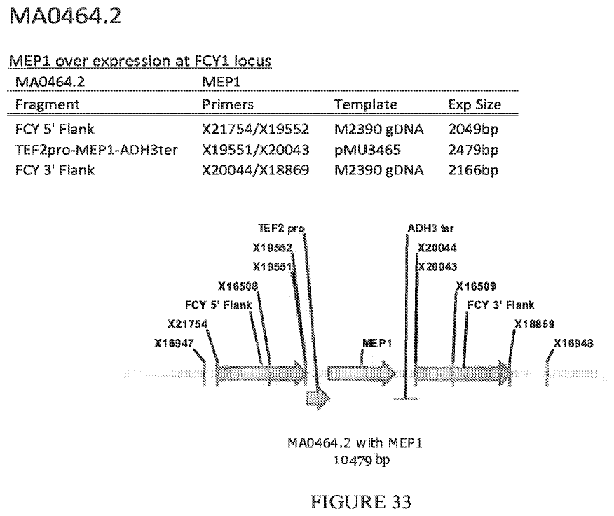

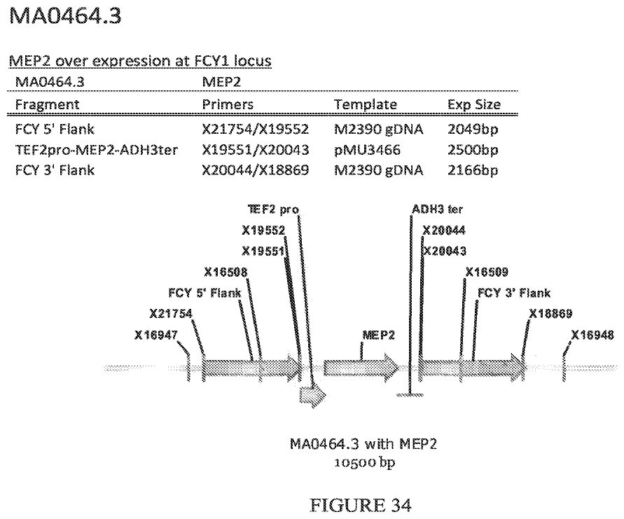

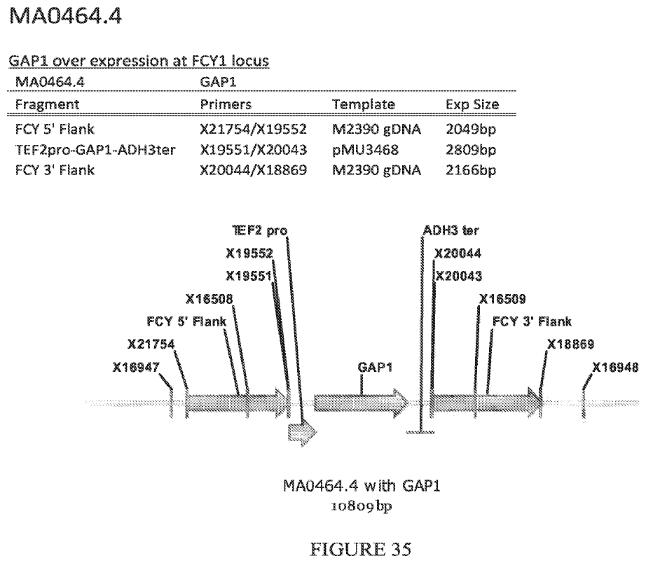

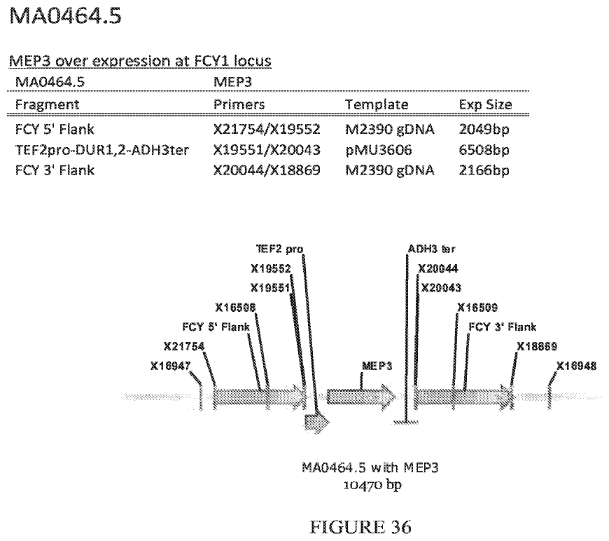

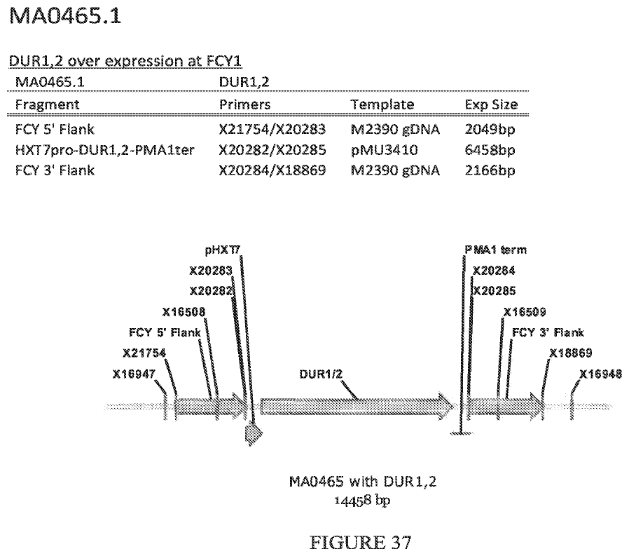

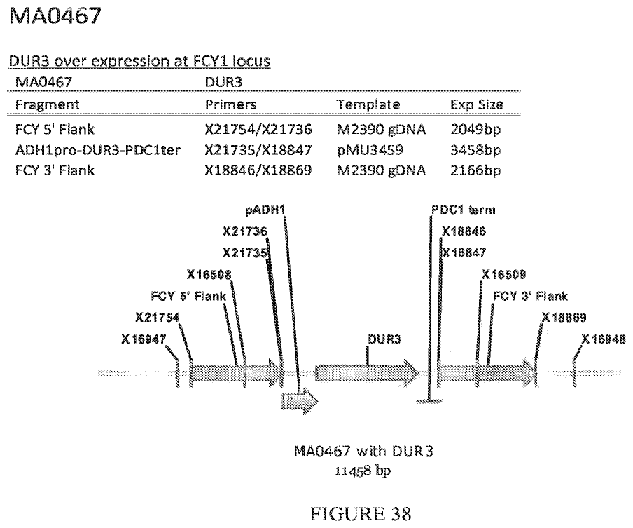

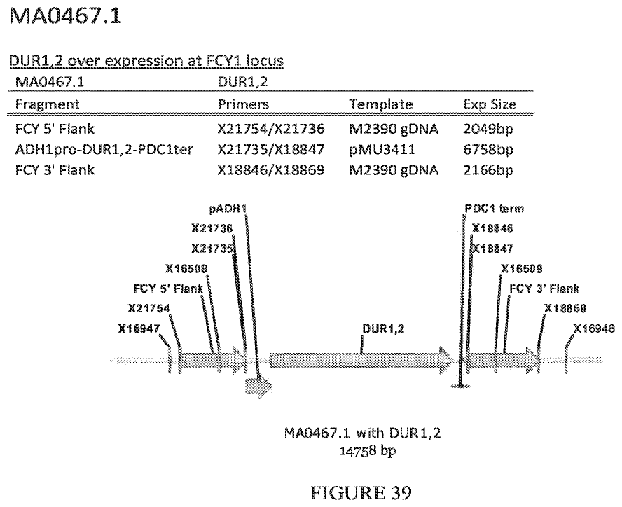

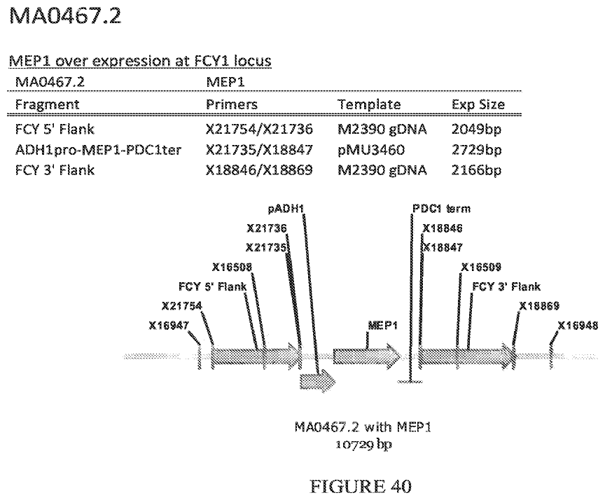

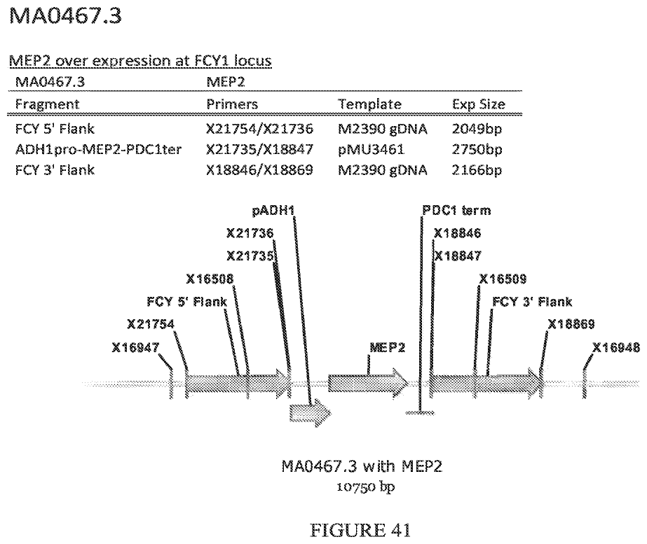

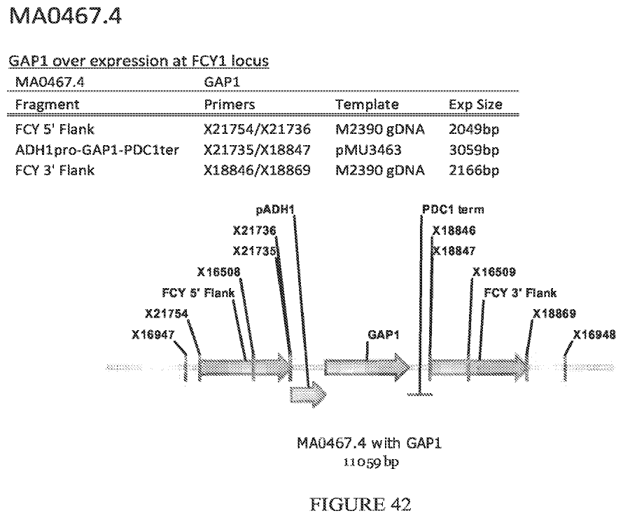

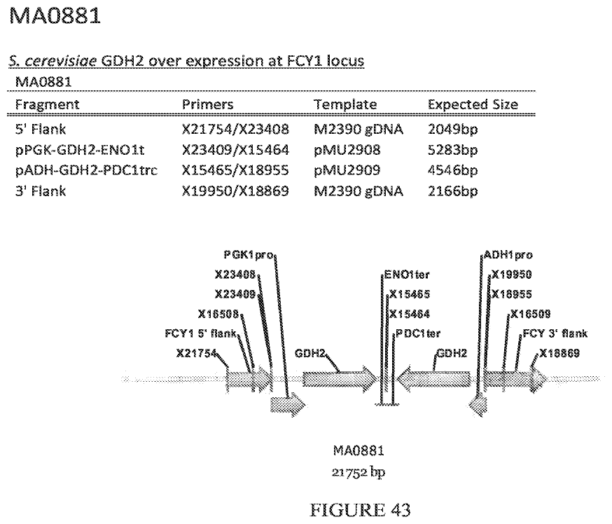

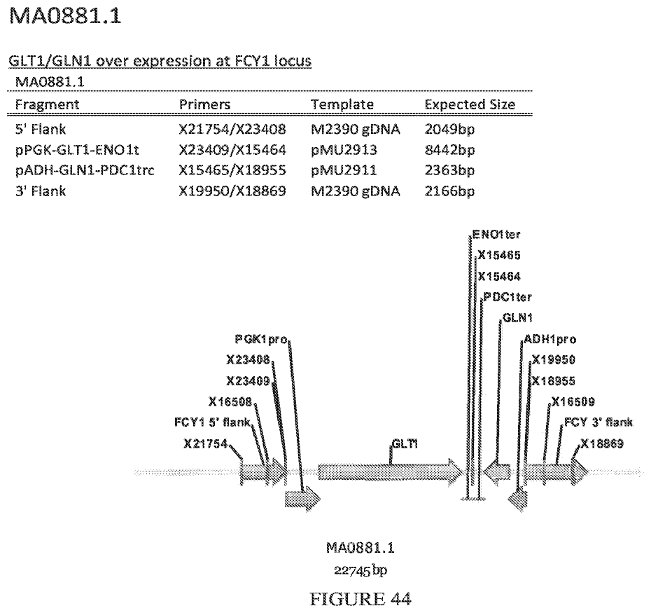

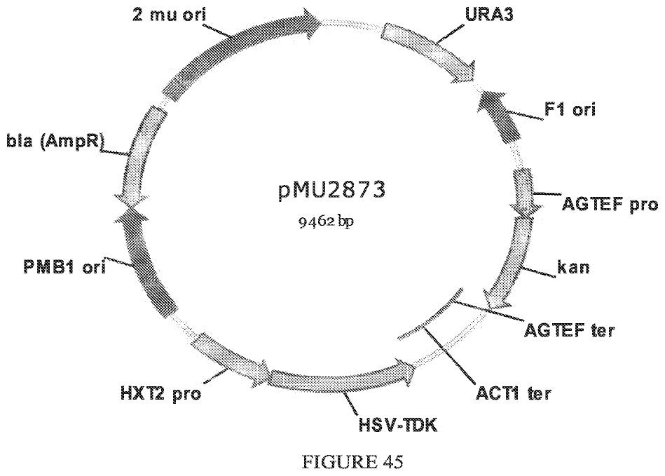

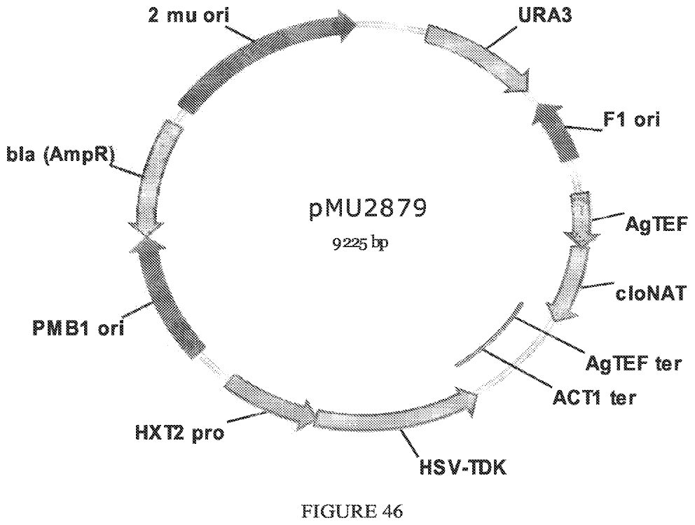

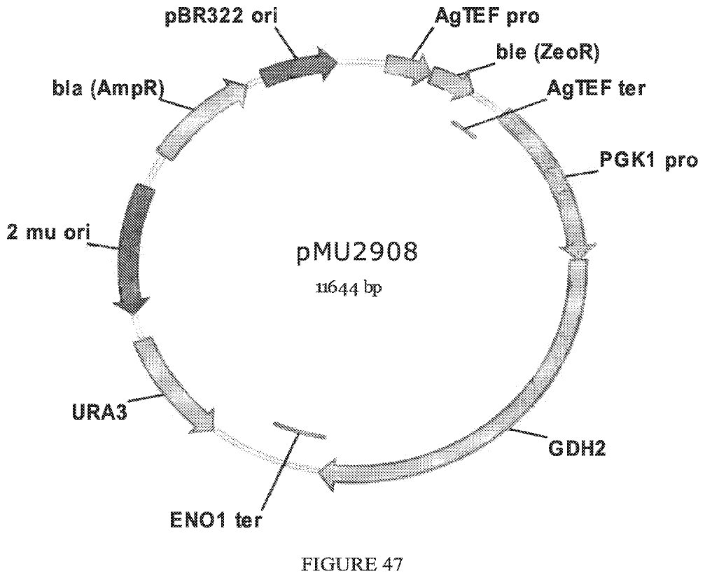

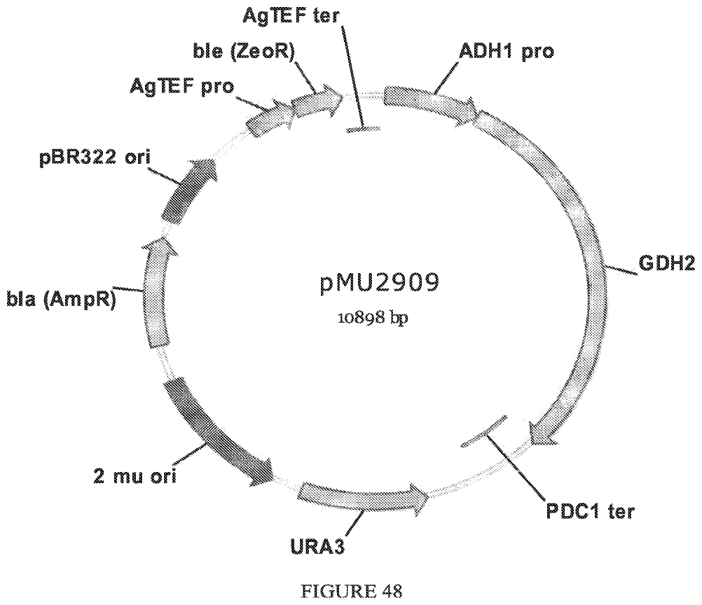

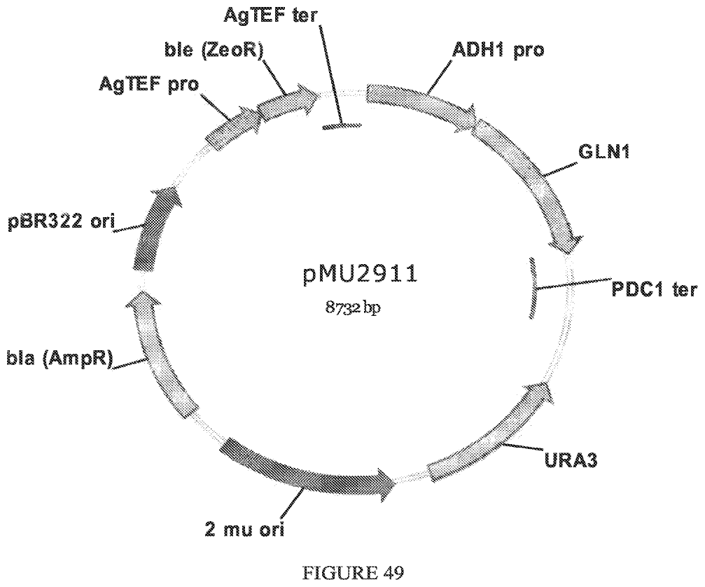

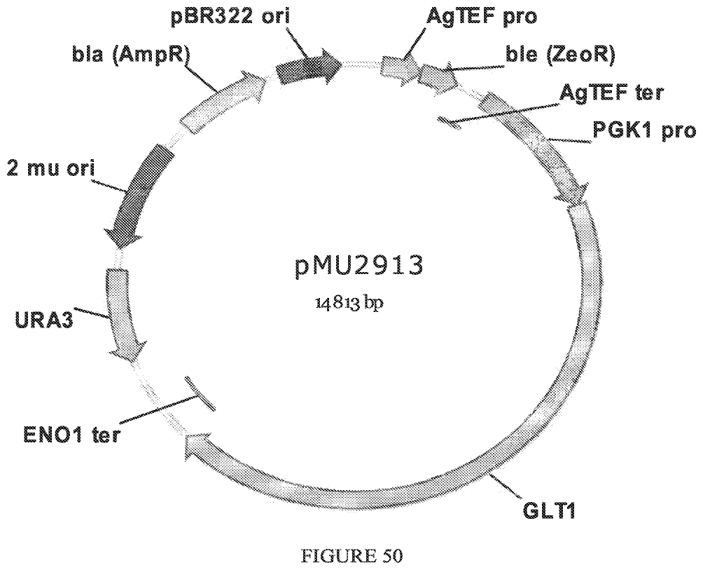

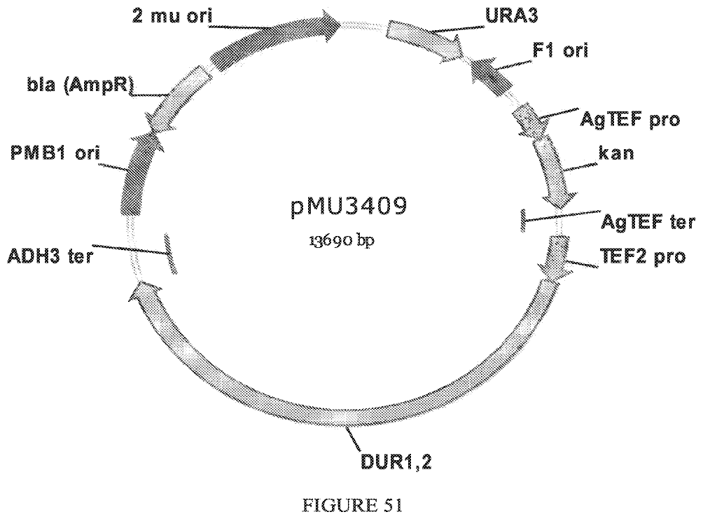

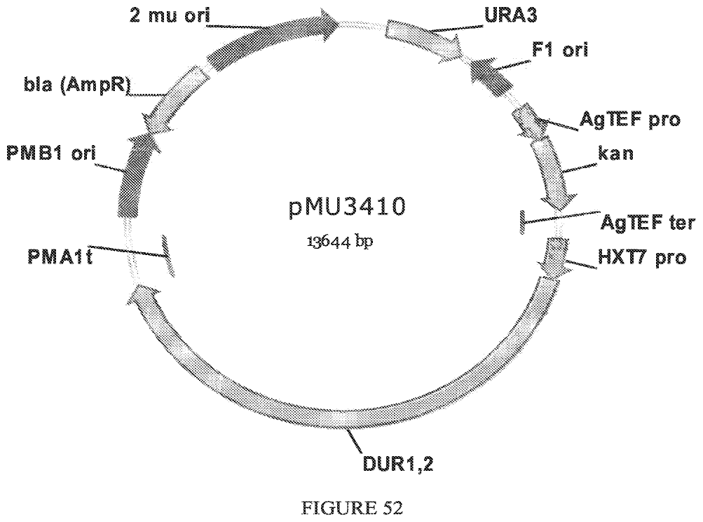

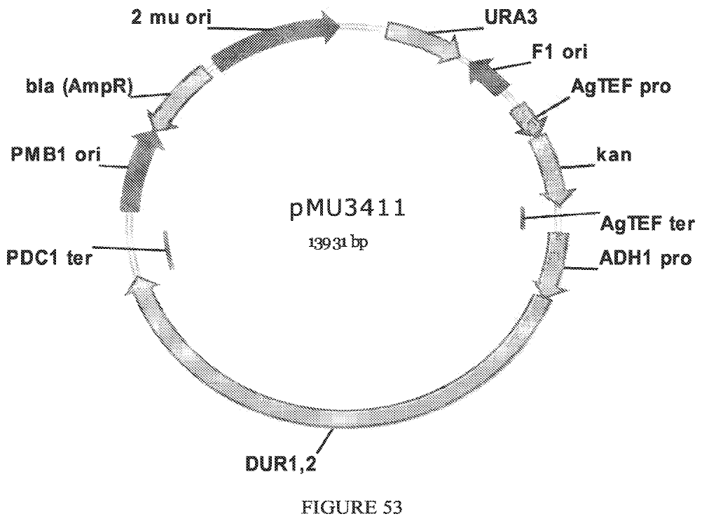

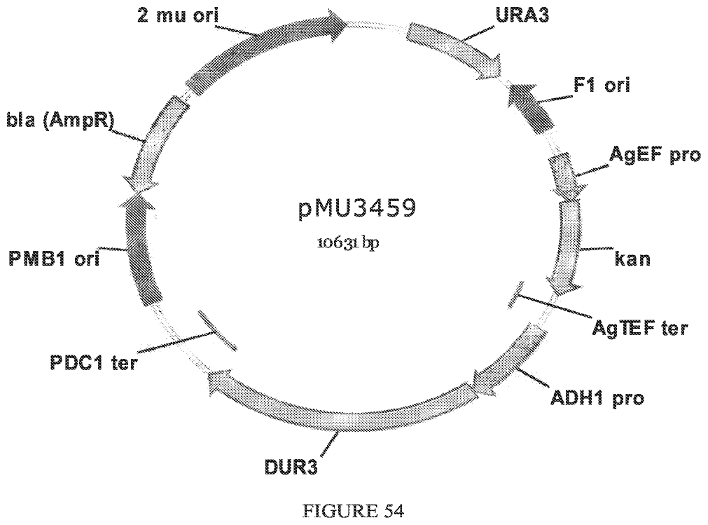

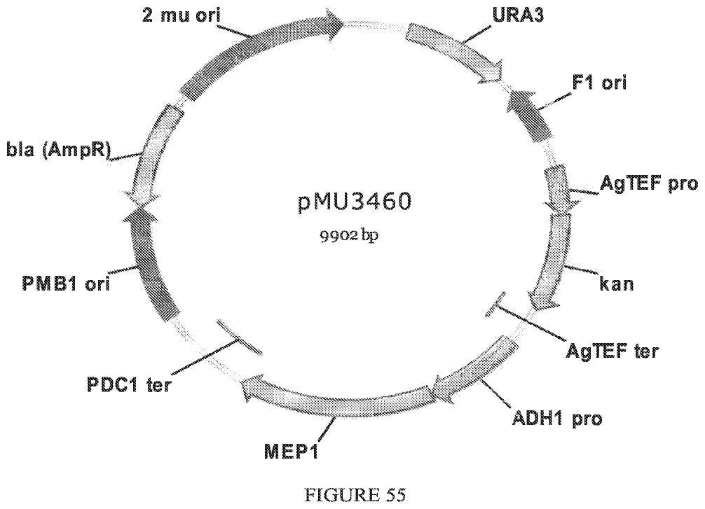

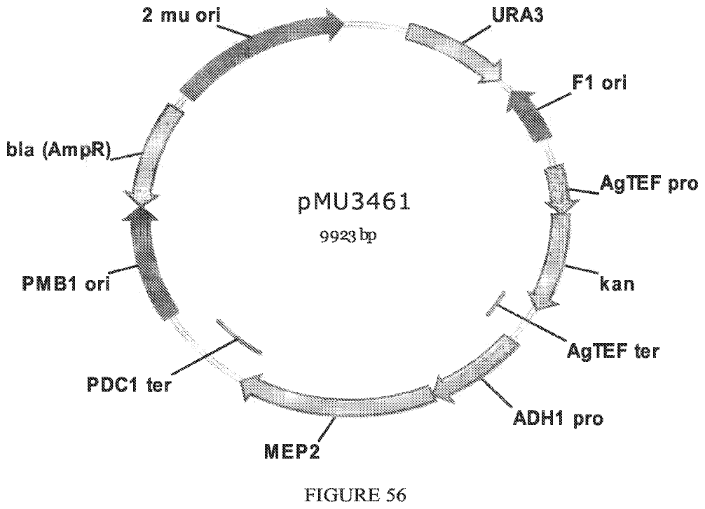

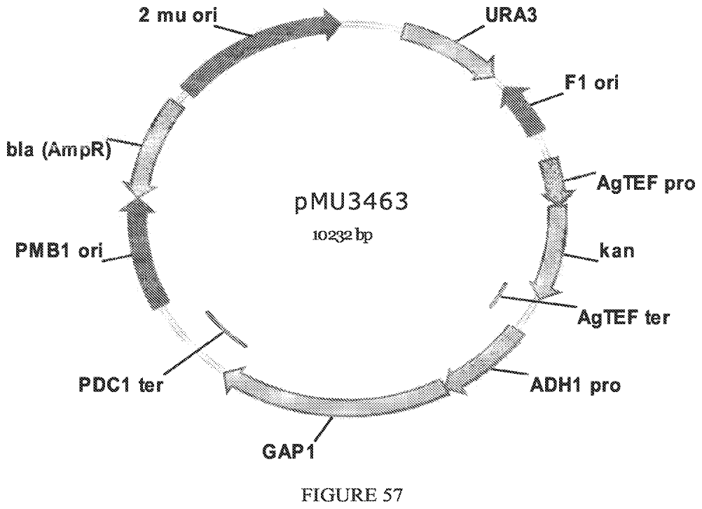

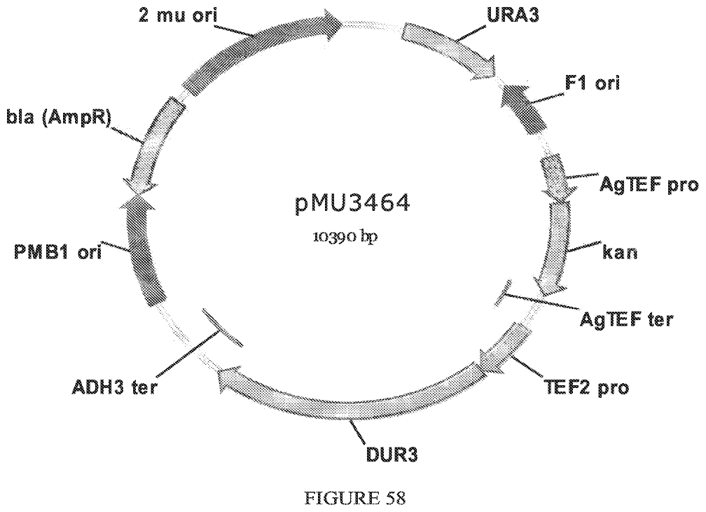

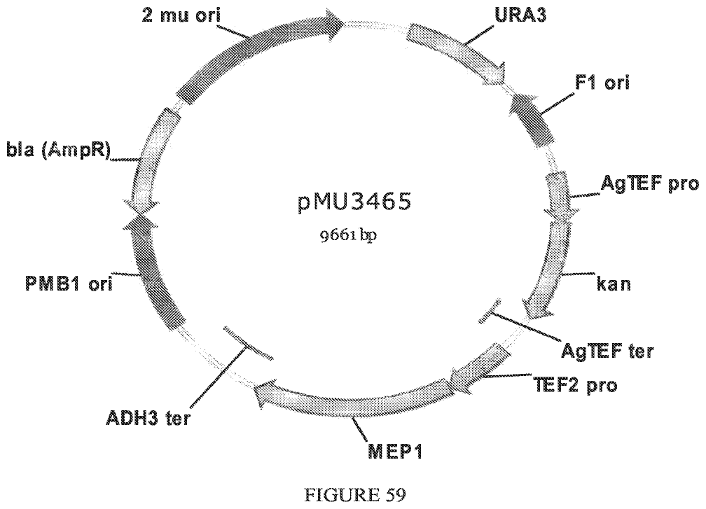

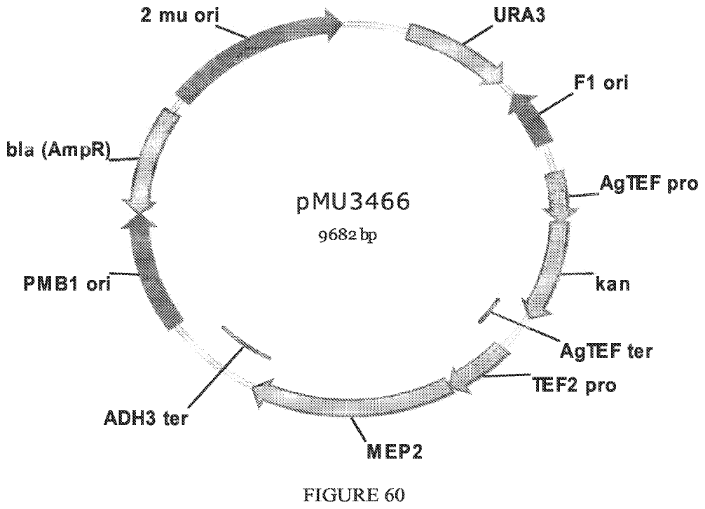

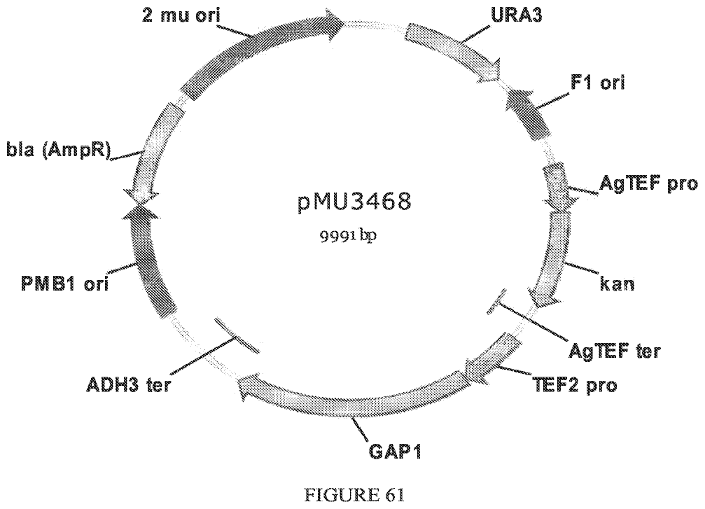

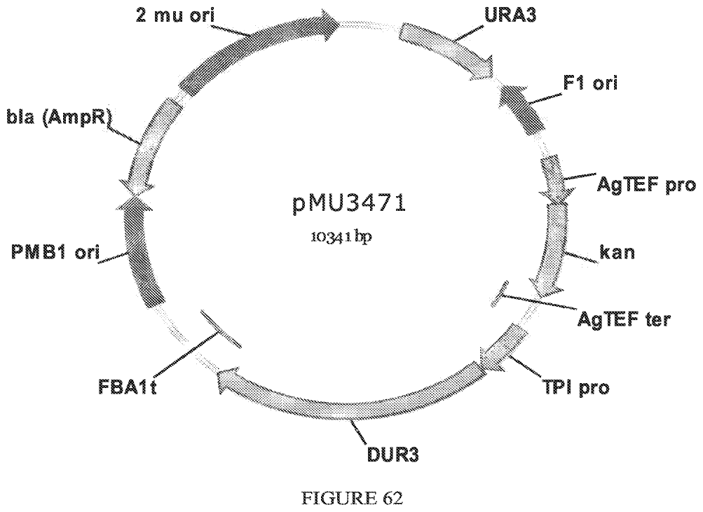

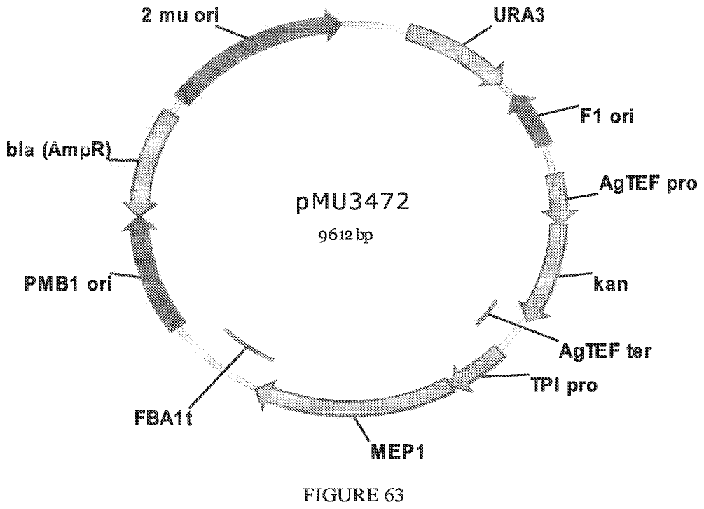

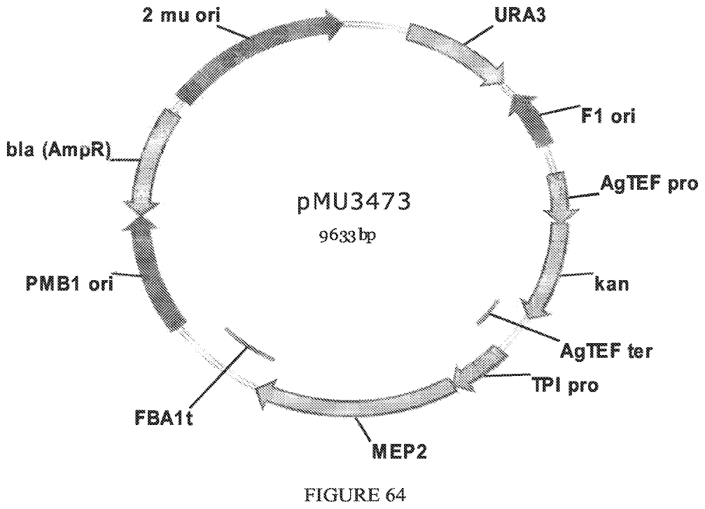

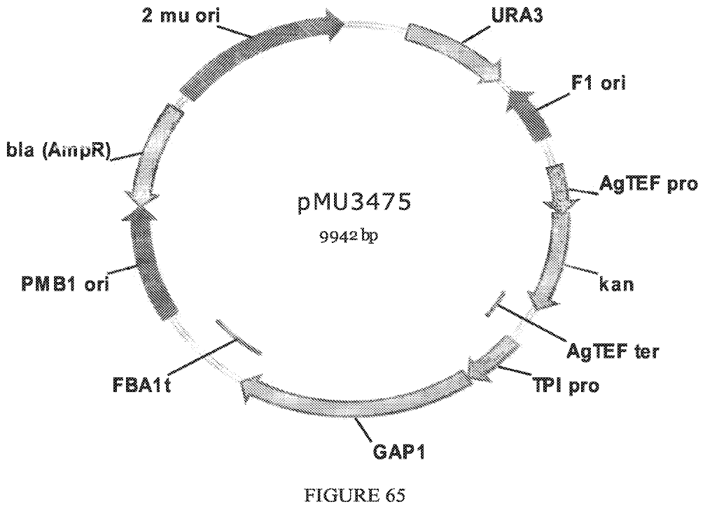

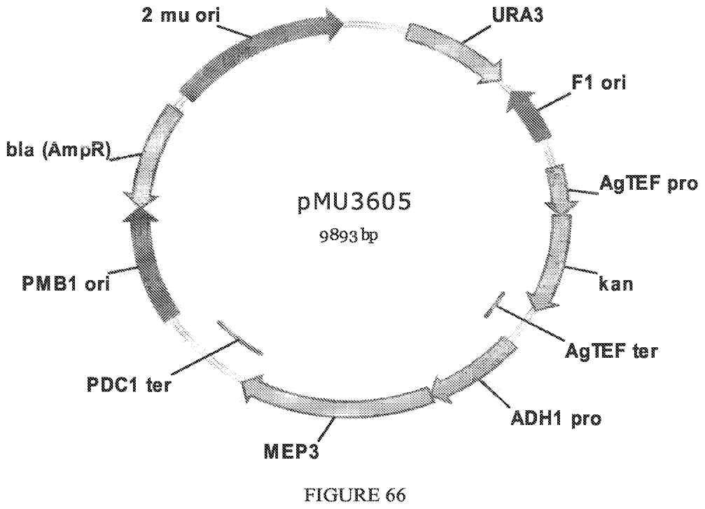

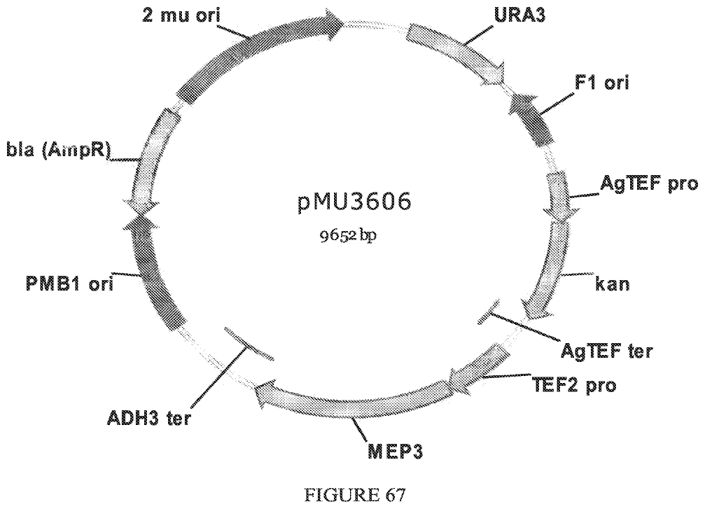

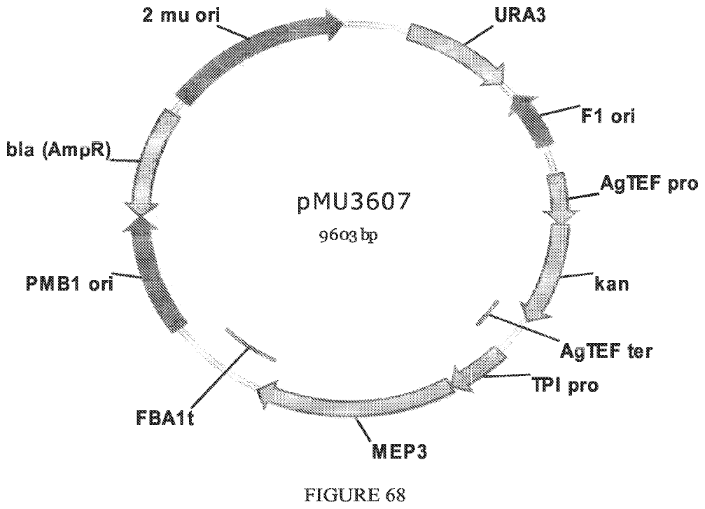

Strains used in the following examples were created using Mascoma Assemblies (“MAs”). Schematic diagrams of the MAs can be seen in FIGS. 6-44. Plasmids used to make the MAs can be seen in FIGS. 45-68 and Table 2. Primers used to create the MAs can be seen in Table 3 below and in SEQ ID NOs: 66-155. Strains used in the invention can be seen in Table 4 below. For a general description of molecular methods that could be used to create the strains, see U.S. Application No. 61/728,450, which is incorporated herein by reference.

TABLE 2
Plasmids used to make the MAs.

| Plasmid ID | Description |
|---|---|
| pMU2873 | AGTEF pro-KAN-AGTEF ter/HXT2 pro-TDK-ACT1 ter |
| pMU2879 | AGTEF pro-cloNAT-AGTEF ter/HXT2 pro-TDK-ACT1 ter |
| pMU2908 | PGK1 pro-S. cerevisiae GDH2-ENO1 ter |
| pMU2909 | ADH1 pro-S. cerevisiae GDH2-PDC1 ter |
| pMU2911 | ADH1 pro-GLN1-PDC1 ter |
| pMU2913 | PGK1 pro-GLT1-ENO1 ter |
| pMU3409 | TEF2 pro-DUR1, 2-ADH3 ter |
| pMU3410 | HXT7 pro-DUR1, 2-PMA1t |
| pMU3411 | ADH1 pro-DUR1, 2-PDC1 ter |
| pMU3459 | ADH1 pro-DUR3-PDC1 ter |
| pMU3460 | ADH1 pro-MEP1-PDC1 ter |
| pMU3461 | ADH1 pro-MEP2-PDC1 ter |
| pMU3463 | ADH1 pro-GAP1-PDC1 ter |
| pMU3464 | TEF2 pro-DUR3-ADH3 ter |
| pMU3465 | TEF2 pro-MEP1-ADH3 ter |
| pMU3466 | TEF2 pro-MEP2-ADH3 ter |
| pMU3468 | TEF2 pro-GAP1-ADH3 ter |
| pMU3471 | TPI pro-DUR3-FBA1 ter |
| PMU3472 | TPI pro-MEP1-FBA1 ter |
| pMU3473 | TPI pro-MEP2-FBA1 ter |
| pMU3475 | TPI pro-GAP1-FBA1 ter |
| pMU3597 | ADH1 pro-N. crassa GDH2-PDC1 ter |
| pMU3605 | ADH1 pro-MEP3-PDC1 ter |
| pMU3606 | TEF2 pro-MEP3-ADH3ter |
| pMU3607 | TPI1 pro-MEP3-FBA1 ter |

TABLE 3
Primers used to create MAs.

| SEQ | | | |
|---|---|---|---|
| ID | Primer | Sequence 5′ to 3′ | Description |
| 66 | X14961 | gcagttacctttagcacccaac | 5′ GDH1 5′ flank |
| 67 | X14966 | ggtgtaggtaagcagaatgaggag | 3′ GDH1 3′ flank |
| 68 | X15464 | GTCCATGTAAAATGATTGCTCCAATGATTGAAAGAGGTTTAGACATTGGCTCTTCATTG | ENOt + PDC1t |
| 69 | X15465 | ctaagctcaatgaagagccaatgtctaaacctctttcaatcattggagcaatcatttta | PDC1t + ENOt |
| 70 | X18846 | gtccatgtaaaatgattgctccaatgattgaaaagcacgcagcacgctgtatttacgtat | FCY3′ + PDC1t |
| 71 | X18847 | AATTAAATACGTAAATACAGCGTGCTGCGTGCTTTTCAATCATTGGAGCAATCATTTTA | PDC1t + FCY3′ |
| 72 | X18858 | agccagcttaaagagttaaaaatttcatagctactacttattcccttcgagattatatct | pTP1 + FCY5′ |
| 73 | X18859 | GTTCCTAGATATAATCTCGAAGGGAATAAGTAGTAGCTATGAAATTTTTAACTCTTTAA | FCY5′ + pTP1 |
| 74 | X18860 | acatcatcttttaacttgaatttattctctagcagcacgcagcacgctgtatttacgtat | FCY3′ + FBA1t |
| 75 | X18861 | AATTAAATACGTAAATACAGCGTGCTGCGTGCTGCTAGAGAATAAATTCAAGTTAAAAG | FBA1t + FCY3′ |
| 76 | X18869 | AGATCCTGTGGTAGTGCTGTCTGAACAGAA | FCY3′ for 2kb |
| 77 | X18955 | ataaaattaaatacgtaaatacagcgtgctgcgtgctcgattttttttctaaacgtgga | pADH1 + FCY3′ rev |
| 78 | X19513 | acttggtgcggtccatgtaaaatgattgctccaatgattgaaaatgaggagaaatccaa | ADH3t + PDC1t |
| 79 | X19514 | TGAAGGTCATTAGGATTTGGATTTCTTCCTCATTTTCAATCATTGGAGCAATCATTTTAC | PDC1t + ADH3t |
| 80 | X19551 | agccagctaaagagttaaaaatttcatagctagggcgccataaccaaggtatctatag | pTEF2 + FCY5′ |
| 81 | X19552 | TGGCGGTCTATAGATACCTTGGTTATGGCGCCCTAGCTATGAAATTTTTAACTCTTTAAG | FCY5′ + pTEF2 |
| 82 | X19721 | aaagaaatgtcagagccagaatttcaacaagctaagctttctaatgatctatccaaaa | pPGK + GDH15′ |
| 83 | X19722 | TTTTCAGTTTTGGATAGATCAGTTAGAAAGCTTAGCTTGTTGAAATTCTGGCTCTGACAT | GDH15′ + pPGK |
| 84 | X19726 | atccgaaatattccacggtttagaaaaaatcggatgctatgtttgaccaaggtgatgta | GDH13′ + pADH1 |
| 85 | X19727 | TTAAAATACATCACCTTGGTCAAACATAGCATCCGATTTTTTTCTAAACCGTGGAATATT | pADH1 + GDH13′ |
| 86 | X19948 | aaagaaatgtcagagccagaatttcaacaagctgatgctatgtttgaccaaggtgatgta | GDH13′ + GDH15′ for |
| | | | deletion |
| 87 | X19949 | TTAAAATACATCACCTTGGTCAAACATAGCATCAGCTTGTTGAAATTCTGGCTCTGACAT | GDH15′ + GDH13′ for |
| | | | deletion |
| 88 | X19950 | atccgaaatattccacggtttagaaaaaaatcgagcacgcagcacgctgtatttacgtat | FCY3′ + pADH1 |
| 89 | X19967 | tgaaggtcattaggatttggatttcttcctcataaattagtgtgtgtgcattatatatat | PMA1t + ADH3t |
| 90 | X19968 | TTTTTAATATATATAATGCACACACACTAATTTATGAGGAAGAAATCCAAATCCTAATGA | ADH3t + PMA1t |
| 91 | X19969 | AATTAAATACGTAAATACAGCGTGCTGCGTGCTCCAGAAAGGCAACGCAAAATTTTTTT | pHXT7 + FCY3′ |
| 92 | X19970 | ccctggaaaaaaaattttgcgttgcctttctggagcacgcagcacgctgtatttacgtat | FCY3′ + pHXT7 |
| 93 | X20022 | aggtagacgctacagtcacaggtgtcacaact | URE2 5′ flank |
| 94 | X20023 | GGACGAGGCAAGCTAAACAGATCTCTAGACCTATTGGTGTACAACTTAATTTGCAGCTTA | URE2 5′ flank |
| 95 | X20024 | ccgtttcttttctttggactatcatgtagtctcaggctgctttaaaaacaagaaagaaag | URE2 3′ flank |
| 96 | X20025 | GAGTGGGATGCGCATATAGTGCATGAACCTAT | URE2 3′ flank |
| 97 | X20026 | ttgttttaagctgcaaattaagttgtacaccaaaggctgctttaaaaacaagaaagaaag | URE2 3′ + 5′ for deletion |
| 98 | X20027 | CTTCTTCTTTCTTTCTTGTTTTTAAAGCAGCCTTTGGTGTACAACTTAATTTGCAGCTTA | URE2 5′ + 3′ for deletion |
| 99 | X20028 | ttgttttaagctgcaaattaagttgtacaccaataggtctagagatctgtttagcttgcc | pAGTEF + URE2 5′ |
| 100 | X20029 | CTTCTTCTTTCTTTCTTGTTTTTAAAGCAGCCTGAGACTACATGATAGTCCAAAGAAAAG | pHXT2rc + URE2 3′ |
| 101 | X20043 | ATAAAATTAAATACGTAAATACAGCGTGCTGCGTGCTATGAGGAAGAAATCCAAATCCT | ADH3 t tails for FCY1 3′ |
| | | | flank |
| 102 | X20044 | tgaaggtcattaggatttggatttcttcctcatagcacgcagcacgctgtatttacgta | FCY 3′ flank tails for |
| | | | ADH3t |
| 103 | X20282 | agccagcttaaagagttaaaatttcatagctaccagaaaggcaacgcaaaatttttttt | pHXT7 + FCY5′ |
| 104 | X20283 | CCCTGGAAAAAAAATTTTGCGTTGCCTTTTCTGGTAGCTATGAAATTTTTAACTCTTTAAG | FCY5′ + pHXT7 |
| 105 | X20284 | tttttaatatatataatgcacacacactaatttagcacgcagcacgctgtatttacgtat | FCY3′ + PMA1t |
| 106 | X20285 | AATTAAATACGTAAATACAGCGTGCTGCGTGCTAAATTAGTGTGTGTGCATTATATATAT | PMA1t + FCY3′ |
| 107 | X20286 | agccagcttaaagagttaaaaattcatagctatgtggtagaattcaaagactatgtga | pGPM1 + FCY5′ |
| 108 | X20287 | ATGGCATCACATAGTCTTTTGAATTCTACCACATAGCTATGAAATTTTTAACTCTTTAAG | FCY5′ + pGPM1 |
| 109 | X20288 | ttttaatattgcttttcaattactgttattaaaagcacgcagcacgctgtatttacgtat | FCY3′ + TPIt |
| 110 | X20289 | AATTAAATACGTAAATACAGCGTGCTGCGTGCTTTTAATAACAGTAATTGAAAAGCAATA | TPIt + FCY3′ |
| 111 | X20620 | ggtgattggaatggttatggttccggaatcgc | AUA1 5′ Flank |
| 112 | X20621 | GGACGAGGCAAGCTAAACAGATCTCTAGACCTATATACTACATAGAAAGCAATTAAAAGA | AUA15′ + pAGTEF |
| 113 | X20622 | ccgtttcttttctttggactatcatgtagtctcctccacctaacaaacccgcaccaacac | AUA13′ + pHXT2rc |
| 114 | X20623 | GTCATATGGCCTCTTAACGTGGTCCTTTGTGG | AUAI 3′ Flank |
| 115 | X20630 | tttttatcttttaattgctttctatgtagtatataggtctagagatctgtttagcttgcc | pAGTEF + AUA1 5′ |
| 116 | X20631 | TACTTGGTGTTGGTGCGGGTTTGTTAGGTGGAGGAGACTACATGATAGTCCAAAGAAAAG | pHXT2rc + AUA13′ |
| 117 | X20632 | tttttatcttttaattgctttctatgtagtatactccacctaacaaacccgcaccaacac | AUA13′ + AUA15′ |
| 118 | X20633 | TACTTGGTGTTGGTGCGGGTTTGTTAGGTGGAGTATACTACATAGAAAGCAATTAAAAGA | AUA15′ + AUA13′ |
| 119 | X21123 | gcgacatgtgatgagattgcatgcacctccacagaa | GDH2 5′ Flank |
| 120 | X21124 | GGACGAGGCAAGGCTAAACAGATCTCTAGACCTATCTTTATTCTTTTTATTGTTGTGAATT | GDH2 5′ Flank + pAGTEF |
| 121 | X21125 | ccgtttcttttctttggactatcatgtagtctcgcttcaataaaattgttttgtataaat | GDH2 3′ Flank + pHXT2rc |
| 122 | X21126 | GGCAGCTATCTCTACTATCCCGTTTAGTACTATCC | GDH2 3′ Flank |
| 123 | X21127 | atattaaattcacaacaataaaaagaataaagataggtctagagatctgtttagcttgcc | pAGTEF + GDH2 5′ |
| 124 | X21128 | GAACTAATTTATACAAAACAATTTTATTGAAGCGAGACTACATGATAGTCCAAAGAAAAG | pHXT2rc + GDH2 3′ |
| 125 | X21133 | atattaaattcacaacaataaaaagaataaagagcttcaataaaattgttttgtataaa | GDH2 3′ + GDH2 5′ for |
| | | | deletion |
| 126 | X21135 | gcattgattgtctatcagagcatatcaaggtggt | GDH3 5′ Flank |
| 127 | X21136 | GGACGAGGCAAGCTAAACAGATCTCTAGACCTACGGTGACTGTTGCTACTTCCCTATATA | GDH3 5′ Flank + pAGTEF |
| 128 | X21137 | ccgtttcttttctttggactatcatgtagtctcccgtaagcgcctattttctttttgttcg | GDH3 3′ Flank + pHXT2rc |
| 129 | X21138 | GGCTAGGACCCGTAAGGAGGAAAGAATAGGCAAG | GDH3 3′ Flank |
| 130 | X21139 | tatatatatatagggaagtagcaacagtcaccgtaggtctagagatctgtttagcttgcc | pAGTEF + GDH3 5′ |
| 131 | X21140 | TAGTTACGAACAAAAAGAAAATAGCGCTTACGGGAGACTACATGATAGTCCAAAGAAAAG | pHXTrc + GDH3 3′ |
| 132 | X21147 | tatatatatatagggaagtagcaacagtcaccgccgtaagcgctattttctttttgttcg | GDH3 3′ + GDH3 5′ |
| 133 | X21148 | TAGTTACGAACAAAAAGAAAATAGCGCTTACGCGGTGACTGTTGCTACTTCCCTATATA | GDH3 5′ + GDH3 3′ |
| 134 | X21179 | ttgttttaagctccaaattaagttgtacaccaagggcgccataaccaaggtatctataga | pTEF2 + URE2 5′ |
| 135 | X21180 | TGGCGGTCTATAGATACCTTGGTTATGGCGCCCTTGGTGTACAACTTAATTTGCAGCTTA | URE2 5′ + pTEF2 |
| 136 | X21181 | CTTCTTCTTTCTTTCTTGTTTTTAAAGCAGCCTCGATTTTTTTCTAAACCGTGGAATATT | pADH1rc + URE2 3′ |
| 137 | X21182 | atccgaaatattccacggtttagaaaaaaatcgaggctgctttaaaaacaagaaagaaag | URE2 3′ + pADH1rc |
| 138 | X21289 | aaagaaatgtcagagccagaatttcaacaagctaggtctagagatctgtttagcttgcct | pAGTEF + GDH1 5′ |
| 139 | X21290 | TTAAAATACATCACCTTGGTCAAACATAGCATCGAGACTACATGATAGTCCAAAGAAAAG | pHXT2rc + GDH1 3′ |
| 140 | X21291 | GGGACGAGGCAAGCTAAACAGATCTCTAGACCTAGCTTGTTGAAATTCTGGCTCTGACAT | GDH1 5′ + pAGTEF |
| 141 | X21292 | ccgtttcttttctttggactatcatgtagtctcgatgctatgtttgaccaaggtgatgt | GDH1 3′ + pHXT2rc |
| 142 | X21319 | ttgttttaagctgcaaaattaagttgtacaccaacgatttttttctaaaccgtggaatatt | pADH1 + URE2 5′ |
| 143 | X21320 | ATCCGAAATATTCCACGGTTTAGAAAAAATCGTTGGTGTACAACTTAATTTGCAGCTTA | URE2 5′ + pADH1 |
| 144 | X21321 | ttttcagttttggatagatcagttagaaagcttaggctgctttaaaaacaagaaagaaag | URE2 3′ + PGKprc |
| 145 | X21322 | CTTCTTCTTTTCTTTTCTTGTTTTTAAAGCAGCCTAAGCTTTCTAACTGATCTATCCAAAAC | PGKprc + URE2 3′ |
| 146 | X21507 | GAACTAATTTATACAAAACAATTTTATTGAAGCTCTTTATTCTTTTTATTGTTGTGAATT | GDH25′ + GDH23′ for |
| | | | deletion |
| 147 | X21735 | agccagctaaaagagttaaaaatttcatagctacgatttttttctaaacgtggaatatt | pADH1 + FCY5′ |
| 148 | X21736 | ATCCGAAATATTCCACGGTTTAGAAAAAAATCGTAGCTATGAAATTTTTAACTCTTTAAG | FCY5′ + pADH1 |
| 149 | X21754 | gccaaagtggattctccactcaagctttgc | FCY5′ Flank |
| 150 | X23319 | aaagaaatgtcagagccagaatttcaacaagctcgatttttttctaaaccgtggaatatt | pADH1 + GDH15′ |
| 151 | X23320 | ATCCGAAATATTCCACGGTTTAGAAAAAAATCGAGCTTGTTGAAATTCTGGCTCTGACAT | GDH15′ + pADH1 |
| 152 | X23321 | gtccatgtaaaatgattgctccaatgattgaaagatgctatgtttgaccaaggtgatgta | GDH1 3′ + PDC1t |
| 153 | X23322 | TTAAAATACATCACCTTGGTCAAACATAGCATCTTTCAATCATTGGAGCAATCATTTTAC | PDC1t + GDH13′ |
| 154 | X23408 | TTTTCAGTTTTGGATAGATCAGTTAGAAAGCTTTAGCTATGAAATTTTTAACTCTTTAAG | FCY5′ + pPGK |
| 155 | X23409 | agccagcttaaagagttaaaaatttcatagctaaagctttctaactgatctatccaaaac | pPGK + FCY5′ |

TABLE 4
Strain genotypes

| Strain | Description | Genotype | Associated MA Cassette(s) |
|---|---|---|---|
| M2390 | Type Strain | WT | |
| M3465 | Glycerol Reduction Strain | Δfdh1::MA0608Δfdh2::MA0280Δgpd2::MA0289 | MA0280, MA0289, MA0608 |
| M3467 | Glycerol Reduction Strain | Δfdh1::MA0608Δfdh2::MA0280Δgpd1::MA0290 | MA0280, MA0290, MA0608 |
| M3469 | Glycerol Reduction Strain | Δfdh1::MA0608Δfdh2::MA0280Δgpd1::MA0293 | MA0280, MA0608, MA0293 |
| M3624 | Glycerol Reduction Strain | Δfdh1::MA0608Δfdh2::MA0280Δgpd1::MA0290Δgpd2::MA0286 | MA0286, MA0280, MA0290, MA0608 |
| M4076 | GDH1 marked deletion | Δfdh1::MA0608Δfdh2::MA0280Δgpd1::MA0290Δgpd2::MA0286Δgdh1::MA0631 | MA0286, MA0280, MA0290, MA0608, MA0631 |
| M4117 | S. cerevisiae GDH2 over | Δfdh1::MA0608Δfdh2::MA0280Δgpd1::MA0290Δgpd2::MA0286Δgdh1::MA0425 | MA0286, MA0280, MA0290, MA0608, MA0425 |
| | expression | | |
| M4118 | S. cerevisiae GLN1/GLT1 over | Δfdh1::MA0608Δfdh2::MA0280Δgpd1::MA0290Δgpd2::MA0286Δgdh1::MA0426 | MA0286, MA0280, MA0290, MA0608, MA0426 |
| | expression | | |
| M4312 | URE2 marked deletion | Δfdh1::MA0608Δfdh2::MA0280Δgpd1::MA0290Δgpd2::MA0286Δure2::MA0622 | MA0286, MA0280, MA0290, MA0608, MA0622 |
| M4373 | GDH1 marked deletion | Δfdh1::MA0608Δfdh2::MA0280Δgpd2::MA0289Δgdh1::MA0631 | MA0280, MA0289, MA0608, MA0631 |
| M4375 | GDH1 marked deletion | Δfdh1::MA0608Δfdh2::MA0280Δgpd1::MA0290Δgdh1::MA0631 | MA0280, MA0290, MA0608, MA0631 |
| M4377 | GDH1 marked deletion | Δfdh1::MA0608Δfdh2::MA0280Δgpd1::MA0293Δgdh1::MA0631 | MA0280, MA0608, MA0293, MA0631 |
| M4400 | GDH1 clean deletion | Δfdh1::MA0608Δfdh2::MA0280Δgpd2::MA0289Δgdh1::MA0888 | MA0280, MA0289, MA0608, MA0888 |
| M4401 | GDH1 clean deletion | Δfdh1::MA0608Δfdh2::MA0280Δgpd1::MA0290Δgdh1::MA0888 | MA0280, MA0290, MA0608, MA0888 |
| M4402 | GDH1 clean deletion | Δfdh1::MA0608Δfdh2::MA0280Δgpd1::MA0293Δgdh1::MA0888 | MA0280, MA0608, MA0293, MA0888 |
| M4406 | URE2 clean deletion | Δfdh1::MA0608Δfdh2::MA0280Δgpd1::MA0290Δgdh2::MA0286Δure2::MA0622.1 | MA0286, MA0280, MA0290, MA0608, MA0622.1 |
| M4427 | DUR1, 2 over expression | Δfdh1::MA0608Δfdh2::MA0280Δgpd1::MA0290Δfcy1::MA0464.1 | MA0280, MA0290, MA0608, MA0464.1 |
| M4428 | DUR1, 2 over expression | Δfdh1::MA0608Δfdh2::MA0280Δgpd1::MA0290Δfcy1::MA0465.1 | MA0280, MA0290, MA0608, MA0465.1 |
| M4429 | DUR1, 2 over expression | Δfdh1::MA0608Δfdh2::MA0280Δgpd1::MA0290Δfcy1::MA0467.1 | MA0280, MA0290, MA0608, MA0467.1 |
| M4430 | DUR1, 2 over expression | Δfdh1::MA0608Δfdh2::MA0280Δgpd1::MA0290Δfcy1::MA0454.14 | MA0280, MA0290, MA0608, MA0454.14 |
| M4431 | DUR1, 2 over expression | Δfdh1::MA0608Δfdh2::MA0280Δgpd1::MA0293Δfcy1::MA0464.1 | MA0280, MA0293, MA0608, MA0464.1 |
| M4432 | DUR1, 2 over expression | Δfdh1::MA0608Δfdh2::MA0280Δgpd1::MA0293Δfcy1::MA0465.1 | MA0280, MA0293, MA0608, MA0465.1 |
| M4433 | DUR1, 2 over expression | Δfdh1::MA0608Δfdh2::MA0280Δgpd1::MA0293Δfcy1::MA0467.1 | MA0280, MA0290, MA0608, MA0467.1 |
| M4434 | DUR1, 2 over expression | Δfdh1::MA0608Δfdh2::MA0280Δgpd1::MA0293Δfcy1::MA0454.14 | MA0280, MA0290, MA0608, MA0454.14 |
| M4469 | S. cerevisiae GDH2 over | Δfdh1::MA0608Δfdh2::MA0280Δgpd2::MA0289Δgdh1::MA0425 | MA02S0, MA0289, MA0608, MA0425 |
| | expression | | |
| M4471 | S. cerevisiae GDH2 over | Δfdh1::MA0608Δfdh2::MA0280Δgpd1::MA0293Δgdh1::MA0425 | MA0280, MA0608, MA0293, MA0425 |
| | expression | | |
| M4507 | AUA1 marked deletion | Δfdh1::MA0608Δfdh2::MA0280Δgpd1::MA0290Δgdh2::MA0486Δaua1::MA0617 | MA0286, MA0280, MA0290, MA0608, MA0617 |
| M4538 | GDH1 marked deletion | Δgdh1::MA0631 | MA0631 |
| M4540 | AUA1 marked deletion | Δaua1::MA0617 | MA0617 |
| M4542 | URE2 marked deletion | Δfdh1::MA0608Δfdh2::MA0280Δgpd1::MA0293Δure2::MA0622 | MA0280, MA0608, MA0293, MA0622 |
| M4571 | AUA1 clean deletion | Δfdh1::MA0608Δfdh2::MA0280Δgpd1::MA0290Δgpd2::MA0286Δaua1::MA0617.1 | MA0286, MA0280, MA0290, MA0608, MA0617.1 |
| M4573 | GDH1 clean deletion | Δfdh1::MA0608Δfdh2::MA0280Δgpd1::MA0290Δgpd2::MA0286Δgdh1::MA0888 | MA0286, MA0280, MA0290, MA0608, MA0888 |
| M4590 | GDH1 clean deletion | Δgdh1::MA0888 | MA0888 |
| M4591 | AUA1 clean deletion | Δaua1::MA0617.1 | MA0617.1 |
| M4592 | URE2 clean deletion | Δfdh1::MA0608Δfdh2::MA0280Δgpd1::MA0293Δure2::MA0622.1 | MA0280, MA0608, MA0293, MA0622.1 |
| M4614 | URE2 marked deletion | Δfdh1::MA0608Δfdh2::MA0280Δgpd1::MA0293Δgpd2::MA0286Δgdh1::MA0425Δure2::MA0622 | MA0286, MA0280, MA0290, MA0608, MA0425, MA0622 |
| M4615 | URE2 marked deletion | Δfdh1::MA0608Δfdh2::MA0280Δgpd1::MA0293Δgpd2::MA0286Δgdh1::MA0426Δure2::MA0622 | MA0286, MA0280, MA0290, MA0608, MA0426, MA0622 |
| M4616 | URE2 marked deletion | Δure2::MA0622 | MA0622 |
| M4622 | S. cerevisiae GDH2 over | Δgdh1::MA0425 | MA0425 |
| | expression | | |
| M4623 | S. cerevisiae GLN1/GLT1 over | Δgdh1::MA0426 | MA0426 |
| | expression | | |
| M4624 | S. cerevisiae GLN1/GLT1 over | Δgdh1::MA0426 | MA0426 |
| | expression | | |
| M4625 | S. cerevisiae GLN1/GLT1 over | Δfdh1::MA0608Δfdh2::MA0280Δgpd2::MA0289Δgdh1::MA0426 | MA0280, MA0289, MA0608, MA0426 |
| | expression | | |
| M4625 | S. cerevisiae GLN1/GLT1 over | Δfdh1::MA0608Δfdh2::MA0280Δgpd1::MA0293Δgdh1::MA0426 | MA0280, MA0608, MA0293, MA0426 |
| | expression | | |
| M4626 | S. cerevisiae GLN1/GLT1 over | Δfdh1::MA0608Δfdh2::MA0280Δgpd1::MA0293Δgdh1::MA0426 | MA0280, MA0608, MA0293, MA0426 |
| | expression | | |
| M4654 | GDH3 marked deletion | Δgdh3::MA0615 | MA0615 |
| M4655 | GDH2 marked deletion | Δfdh1::MA0608Δfdh2::MA0280Δgpd1::MA0290Δgpd2::MA0286Δgdh1::MA0426Δgdh2::MA0616 | MA0286, MA0280, MA0290, MA0608, MA0426, MA0616 |
| M4656 | GDH3 marked deletion | Δfdh1::MA0608Δfdh2::MA0280Δgpd1::MA0290Δgpd2::MA0286Δgdh1::MA0426Δgdh3::MA0615 | MA0286, MA0280, MA0290, MA0608, MA0426, MA0615 |
| M4657 | GDH3 marked deletion | Δfdh1::MA0608Δfdh2::MA0280Δgpd1::MA0290Δgpd2::MA0286Δgdh1::MA0425Δgdh3::MA0615 | MA0286, MA0280, MA0290, MA0608, MA0425, MA0615 |
| M4674 | URE2 clean deletion | Δfdh1::MA0608Δfdh2::MA0280Δgpd1::MA0290Δgpd2::MA0286Δgdh1::MA0425Δure2::MA0622.1 | MA0286, MA0280, MA0290, MA0608, MA0425, MA0622.1 |
| M4675 | URE2 clean deletion | Δfdh1::MA0608Δfdh2::MA0280Δgpd1::MA0290Δgpd2::MA0286Δgdh1::MA0426Δure2::MA0622.1 | MA0286, MA0280, MA0290, MA0608, MA0426, MA0622.1 |
| M4676 | URE2 clean deletion | Δure2::MA0622.1 | MA0622.1 |
| M4677 | GDH2 marked deletion | Δgdh2::MA0616 | MA0616 |
| M4690 | GDH3 clean deletion | Δgdh3::MA0615.1 | MA0615.1 |
| M4691 | GDH2 clean deletion | Δfdh1::MA0608Δfdh2::MA0280Δgpd1::MA0290Δgpd2::MA0286Δgdh1::MA0426Δgdh2::MA0616.1 | MA0286, MA0280, MA0290, MA0608, MA0426, MA0616.1 |
| M4692 | GDH3 clean deletion | Δfdh1::MA0608Δfdh2::MA0280Δgpd1::MA0290Δgpd2::MA0286Δgdh1::MA0426Δgdh3::MA0615.1 | MA0286, MA0280, MA0290, MA0608, MA0426, MA0615.1 |
| M4693 | GDH3 clean deletion | Δfdh1::MA0608Δfdh2::MA0280Δgpd1::MA0290Δgpd2::MA0286Δgdh1::MA0425Δgdh3::MA0615.1 | MA0286, MA0280, MA0290, MA0608, MA0425, MA0615.1 |
| M4694 | GDH2 clean deletion | Δgdh2::MA0616.1 | MA0616.1 |
| M4748 | DUR3 over expression | Δfcy1::MA0464 | MA0464 |
| M4749 | GAP1 over expression | Δfcy1::MA0464.4 | MA0464.4 |
| M4750 | MEP1 over expression | Δfcy1::MA0464.2 | MA0464.2 |
| M4751 | DUR1, 2 over expression | Δfcy1::MA0465.1 | MA0465.1 |
| M4752 | MEP1 over expression | Δfcy1::MA0434.2 | MA0434.2 |
| M4753 | MEP2 over expression | Δfcy1::MA0434.3 | MA0434.3 |
| M4754 | DUR3 over expression | Δfcy1::MA0434 | MA0434 |
| M4755 | GAP1 over expression | Δfcy1::MA0434.4 | MA0434.4 |
| M4756 | MEP2 over expression | Δfcy1.MA0464.2 | MA0464.2 |
| M4810 | DUR3 over expression | Δfcy1::MA0467 | MA0467 |
| M4811 | DUR3 over expression | Δfcy1::MA0467 | MA0467 |
| M4812 | MEP1 over expression | Δfcy1::MA0467.2 | MA0467.2 |
| M4813 | MEP1 over expression | Δfcy1::MA0467.2 | MA0467.2 |
| M4814 | GAP1 over expression | Δfcy1::MA0467.4 | MA0467.4 |
| M4815 | GAP1 over expression | Δfcy1::MA0467.4 | MA0467.4 |
| M5841 | N. crassa GDH2 over | Δfdh1::MA0608Δfdh2::MA0280Δgpd1::MA0290Δgpd2::MA0286Δgdh1::MA0837 | MA0280, MA0608, MA0286, MA0290, MA0837 |
| | expression | | |
| M5842 | N. crassa GDH2 over | Δfdh1::MA0608Δfdh2::MA0280Δgpd1::MA0290Δgpd2::MA0286Δgdh1::MA0837 | MA0280, MA0608, MA0286, MA0290, MA0837 |
| | expression | | |
| M5843 | N. crassa GDH2 over | Δfdh1::MA0608Δfdh2::MA0280Δgpd1::MA0290Δgpd2::MA0286Δgdh1::MA0837 | MA0280, MA0608, MA0286, MA0290, MA0837 |
| | expression | | |
| M5844 | N. crassa GDH2 over | Δfdh1::MA0608Δfdh2::MA0280Δgpd1::MA0290Δgpd2::MA0286Δgdh1::MA0837 | MA0280, MA0608, MA0286, MA0290, MA0837 |
| | expression | | |

### Example 1

### Deletion of GDH1 and Overexpression of GDH2 or GLT1/GLN1

M3624 (Δgpd1::GPD2-B. adolescentispf1A/pF1B/adhEΔgpd2::GPD1-B. adolescentispf1A/pf1B/adhEΔfdh1Δfdh2::B. adolescentispf1A/pf1B/adhE) has an approximately 85% reduction in glycerol formation when grown on >30% solids corn mash. However, the strain is unable to complete the fermentation even after extended incubation periods. Two modifications of the ammonium assimilation pathway were constructed in M3624 and evaluated for fermentation performance. The modifications were a deletion of GDH1 and over-expression of Gdh2, resulting in strain M4117 (M3634 Gdh2; Δgdh1). The second modification was a deletion of GDH1 and overexpression of GLT1 and GLN1, resulting in strain M4118 (M3634 Glt1; Gln1; Δgdh1). These strains were compared to M3624 and the conventional yeast control (M2390 (a wild type unmodified strain isolated from industrial sources)) following fermentation of 31% solids corn mash.

An industrial corn mash was prepared to a final solids concentration of 31% supplemented with penicillin (0.006 mg/mL) and urea (0.5 g/l). Glucoamylase was added at a concentration of 0.6 AGU/gTS. Fermentation was stopped by addition of each strain to an final starting concentration of 0.1 g/l. Vials were capped with a rubber stopper and sealed. A 23-gauge needle was inserted through the stopper to vent and for the safety of the experiment. Vials were incubated at 35° C. with shaking at 125 rpm. At the termination of the experiment samples were prepared for HPLC analysis of ethanol and residual sugars.

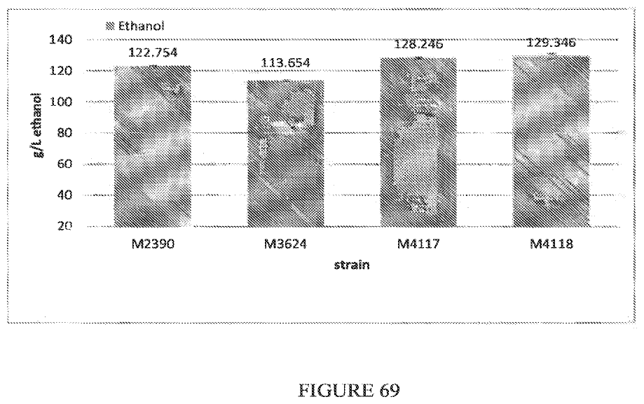

The results in FIG. 69 illustrate that both M4117 and M4118 reach a much higher final ethanol titer than M3624, which was unable to complete the fermentation. Relative to M2390 both M4117 and M4118 had ethanol titers that were 4.2% and 5.2% higher respectively.

### Example 2

### Deletion of GDH1

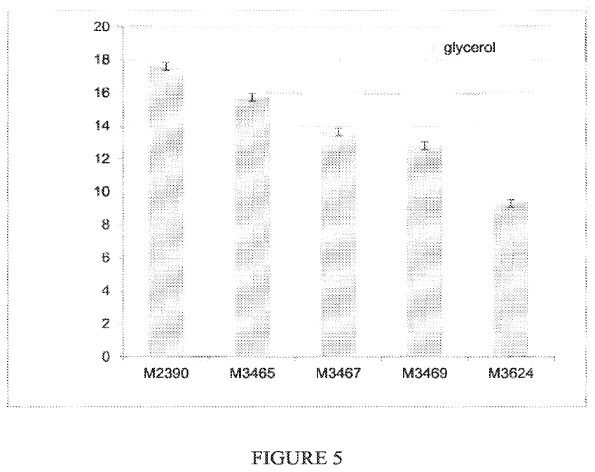

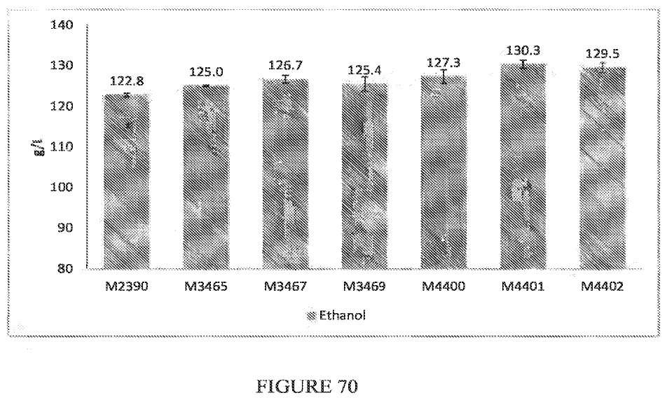

As shown in FIG. 5, M3465 (Δgpd2::B. adolescentispf1A/pf1B/adhEΔfdh1Δ fdh2::B. adolescentispf1A/pf1B/adhE), M3467 (Δfdh1Δfdh2::PFK-pro-adhE-HXT-ter ENO1-pro-pf1B-ENO1-ter ADH1-pro-adhE-PDC10-ter TPI-pro-pf1A-FBA-ter Δgpd1::GPD2::PFK-pro-adhE-HXT-ter ENO1-pro-pf1B-ENO1-ter ADH1-pro-adhE-PDC10-ter TPI-pro-pf1A-FBA-ter) and M3469 (Δgpd1::B. adolescentis pf1A/pf1B/adhE fdh1Δfdh2Δ::B. adolescentispf1A/pf1B/adhE) have degrees of glycerol reduction ranging from 20% to ˜45% relative to the control strain M2390. A clean deletion of GDH1 was constructed in each of these backgrounds resulting in M4400 (M3465 Δgdh1), M4401(M3467 Δgdh1) and M4402 (M3469 Δgdh1) and compared to the conventional yeast control (M2390) following fermentation of 31% solids corn mash (The fermentation was performed as described in Example 1). As shown in FIG. 70, all three glycerol reduction strains engineered with a deletion of GDH1 reached a higher ethanol titer than their respective parent strain.

### Example 3

### Overexpression of DUR1/2

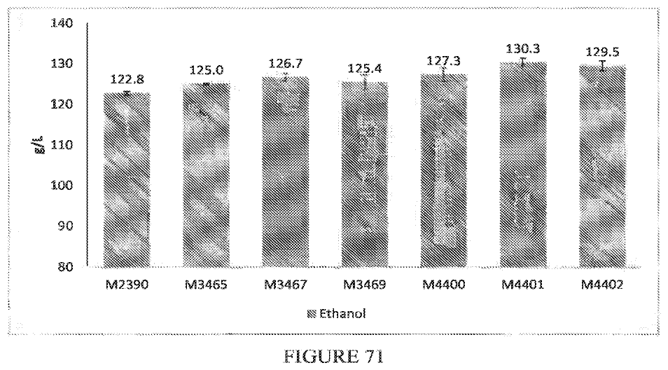

Four different DUR1/2 expression cassettes were constructed in both M3467 Δfdh1Δfdh2::PFK-pro-adhE-HXT-ter ENO1-pro-pf1B-ENO1-ter ADH1-pro-adhE-PDC10-ter TPI-pro-pf1A-FBA-ter Δgpd1::GPD2::PFK-pro-adhE-HXT-ter ENO1-pro-pf1B-ENO1-ter ADH1-pro-adhE-PDC10-ter TPI-pro-pf1A-FBA-ter) and M3469 (Δgpd1::B. adolescentis pf1A/pf1B/adhE fdh1Δfdh2Δ::B. adolescentispf1A/pf1B/adhE) resulting in strains M4427-M3343 (Table 5). These strains were compared to their parent strain and the conventional yeast control (M2390) following fermentation of 31% solids corn mash (The fermentation was performed as described in Example 1). As shown in FIG. 71, all strains containing an overexpression of DUR1/2 reached the same or higher ethanol titers than their respective parent strain but the TEF2 and ADH1 promoter appeared particularly affective. Promoters and terminators used and that could be used include: S. cerevisiae TEF 2 promoter: SEQ ID NO: 58, S. cerevisiae HXT7 promoter: SEQ ID NO: 59, S. cerevisiae, S. cerevisiae ADH1 promoter: SEQ ID NO: 60, S. cerevisiae TP1 promoter: SEQ ID NO: 61, S. cerevisiae FBA1 terminator: SEQ ID NO: 62, S. cerevisiae PDC1 terminator: SEQ ID NO: 63, S. cerevisiae PMA1 terminator: SEQ IS NO: 64, and S. cerevisiae ADH3 terminator: SEQ ID NO: 65.

TABLE 5
Description of constructions and strain designations
containing over-expression of DUR1/2

| Parent | Strain | |
|---|---|---|
| strain | designation | Genetic modification |
| M3467 | M4427 | MA0464.1: expression of DUR1, 2 from |
| | | the TEF2 promoter |
| M3467 | M4428 | MA0465.1: expression of DUR1, 2 from |
| | | the HXT7 promoter |
| M3467 | M4429 | MA467.1: expression of DUR1, 2 from |
| | | the ADH1 promoter |
| M3467 | M4430 | MA454.14: expression of DUR1, 2 from |
| | | the HXT7/TEF2 promoters |
| M3469 | M4431 | MA0464.1: expression of DUR1, 2 from |
| | | the TEF2 promoter |
| M3469 | M4432 | MA0465.1: expression of DUR1, 2 from |
| | | the HXT7 promoter |
| M3469 | M4433 | MA467.1: expression of DUR1, 2 from |
| | | the ADH1 promoter |
| M3469 | M4434 | MA454.14: expression of DUR1, 2 from |
| | | the HXT7/TEF2 promoters |

### Example 4

### Deletion of URE2

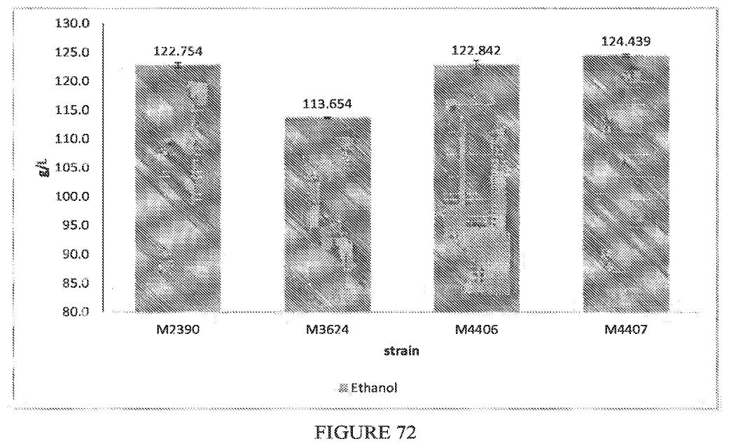

To evaluate an alteration in the S. cerevisiae nitrogen catabolite repression system in glycerol reduction backgrounds, a deletion of URE2 was constructed in M3624 (Example 1), creating strain M4406 (M3624 Δure2). This strain was compared to M3624 and the conventional yeast control (M2390) following fermentation of 31% solids corn mash (The fermentation was performed as described in Example 1). As shown in FIG. 72, M4406 reached a higher titer than M3624 however a yield increase over the conventional strain was not observed and there was −15 g/l residual glucose. This is an indication that additional modifications to the NCR system may give improved performance or that an adaptation of M4406 may be required to obtain the potential yield increase.

### Example 5

### Regulation of Nitrogen Utilization

Preferred nitrogen sources generally repress transcription of genes required to utilize non-preferred nitrogen sources. Urea is added as a supplemental nitrogen source in corn mash fermentation; however, there are significant quantities of amino acids and ammonia, both of which are preferred nitrogen sources over urea. Expression of the urea transporter (Dur3) and the urea:amido lyase responsible for intracellular degradation (Dur1/2) may be repressed in the presence amino acids and ammonia as part of a phenomenon referred to as Nitrogen Catabolite Repression (NCR). This repression could slow the rate of urea uptake or require larger quantities to be added. It would be an economic benefit to a corn ethanol producer if constitutive expression of Dur3 and Dur1,2 allowed them to either reduce the amount of urea needed or accelerate fermentation rate.

The NCR is controlled by Ure2 and four transcription factors known as Gln3, Gat1, Dal80, and Gzf3. Ure2 participates in repressing gene expression in the presence of non-preferred nitrogen source. It has been observed that deletion of URE2 activates the expression of genes involved in the uptake of non-preferred nitrogen sources Inactivation of Ure2 results in dephosphorylation and nuclear localization of the transcription factor Gln3.

### Example 5A

### Deletion of URE2 Deletion of URE2 Results in Nuclear Localization of GLN3 and Activation of NCR Sensitive Genes

To evaluate an alteration in the S. cerevisiae nitrogen catabolite repression system in glycerol reduction backgrounds, a deletion of Ure2 is constructed as in Example 4. A deletion of URE2 will be constructed in M3624 (Example 1). Strains in which URE2 is deleted show a nuclear localization of Gln3, and an activation of NCR sensitive genes, including Dur3 and Dur1/2.

### Example 5B

### Overexpression GLN3 Results in Activation of NCR Sensitive Genes

To evaluate an alteration in the S. cerevisiae nitrogen catabolite repression system in glycerol reduction backgrounds, Gln3 (SEQ ID NOs: 156 and 157) is overexpressed. Strains in which Gln3 is overexpressed show an activation of NCR sensitive genes, including Dur3 and Dur1/2.

### Example 6

### Deletion of GDH1 and Expression of S. cerevisiae GDH2

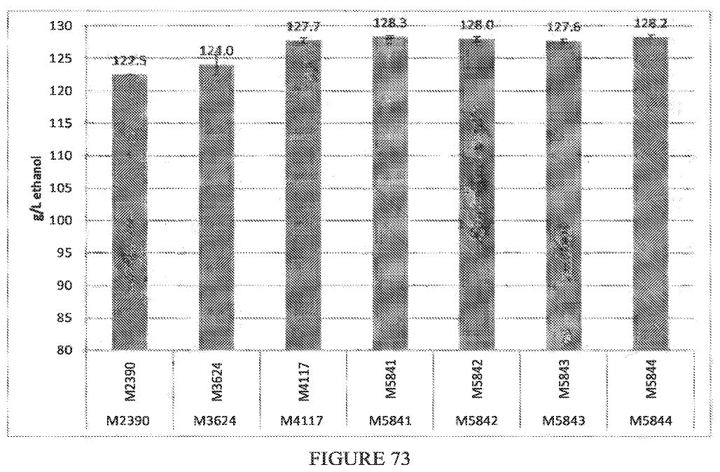

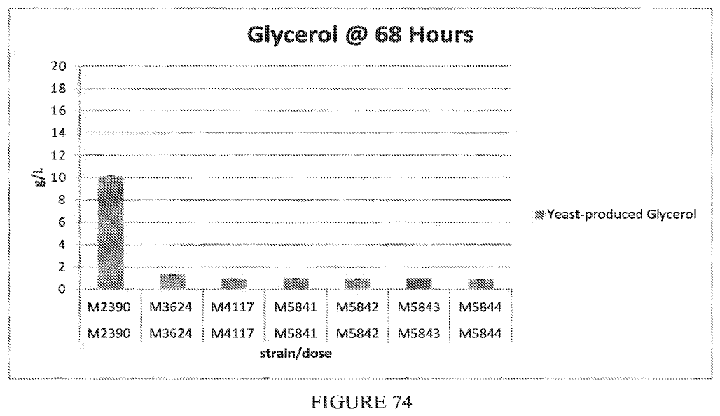

The results show that strain M3624 (Example 1) was able to reach a slightly higher titer than strain M2390 (WT), producing 1.5 g/l more ethanol (FIG. 73). To create strain M4117 (M3634 Gdh2; Δgdh1), the GDH1 gene was deleted and replaced with 4 copies of the S. cerevisiae GDH2 gene expression cassette. The results in FIG. 73 below demonstrate that when compared to M3624, M4117 had a clear improvement of 3.7 g/l more ethanol. The data shown in FIG. 74 shows that M3624 makes 1.3 g/l glycerol which is 87% less than the wild type strain M2390, which made 10 g/l. The deletion of GDH1 and addition of the S. cerevisiae GDH2 expression cassette decreased the glycerol titers to around 1 g/l. These results illustrate that the combination of glycerol reduction through formate production is synergistic with modifications to the ammonium assimilation pathway.

### Example 7

### Deletion of GDH1 and Expression of N. crassa GDH2

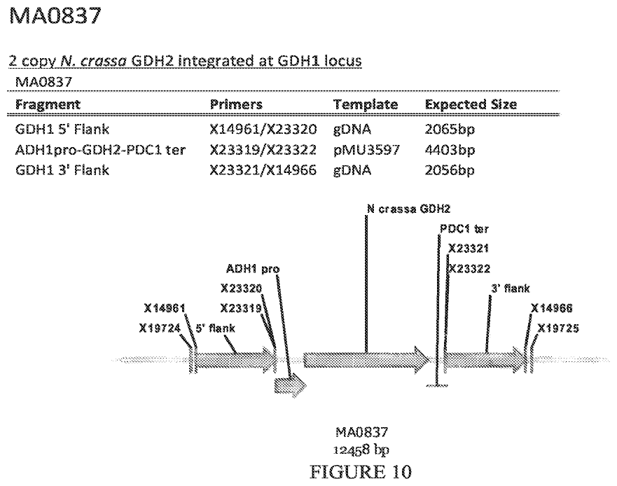

To create strains M5841-M5844, the GDH1 gene was deleted and replaced with 4 copies of the N. crassa GDH2 gene expression cassette in FIG. 10. Each strain resulted from independent colonies and have the same genotype (Table 6). The results shown in FIG. 73 demonstrate that the addition of the N. crassa GDH2 to M3624 resulted in titers that were between 3.6 g/l and 4.3 g/l higher than M3624. The data shown in FIG. 74 shows that M3624 makes 1.3 g/l glycerol which is 87% less than the wild type strain M2390, which made 10 g/l. The deletion of GDH1 and addition of the N. crassa GDH2 expression cassette decreased the glycerol titers to around 1 g/l. These results support the conclusion that a combination of glycerol reduction through formate production is synergistic with modifications to the ammonium assimilation pathway, even when using a heterologous expression of GDH2.

TABLE 6
Glycerol deletion strains which further comprise
a deletion of gdh1 and an expression of Gdh2.

| Strain | Genotype |
|---|---|
| M2390 | WT |
| M3624 | Δfdh1Δfdh2::4a2pΔgpd1::gpd24a2pΔgpd2::gpd14a2p |
| M4117 | Δfdh1Δfdh2::4a2pΔgpd1::gpd24a2pΔgpd2::gpd14a2pΔScgdh1::4gdh2 |
| M5841 | Δfdh1Δfdh2::4a2pΔgpd1::gpd24a2pΔgpd2::gpd14a2pΔNcrassagdh1::4gdh2 |
| M5842 | Δfdh1Δfdh2::4a2pΔgpd1::gpd24a2pΔgpd2::gpd14a2pΔNcrassagdh1::4gdh2 |
| M5843 | Δfdh1Δfdh2::4a2pΔgpd1::gpd24a2pΔgpd2::gpd14a2pΔNcrassagdh1::4gdh2 |
| M5844 | Δfdh1Δfdh2::4a2pΔgpd1::gpd24a2pΔgpd2::gpd14a2pΔNcrassagdh1::4gdh2 |

The strains in Table 6 were inoculated in vials containing 4 ml industrial corn mash (mini-vials). The fermentation was allowed to proceed for 68 hrs and samples were run on an HPLC to obtain ethanol and glycerol values.

All documents cited herein, including journal articles or abstracts, published or corresponding U.S. or foreign patent applications, issued or foreign patents, or any other documents, are each entirely incorporated by reference herein, including all data, tables, figures, and text presented in the cited documents.

Those skilled in the art will recognize, or be able to ascertain using no more than routine experimentation, many equivalents to the specific embodiments of the invention described herein. Such equivalents are intended to be encompassed by the following claims.

1.-215. (canceled)

216. A recombinant yeast comprising:
a) at least one engineered genetic modification that leads to the down-regulation of an enzyme in a glycerol-production pathway; and,
b) at least one engineered genetic modification that leads to the up-regulation or down-regulation of an enzyme in a nitrogen-assimilation pathway;
wherein the down-regulated enzyme in the glycerol-production pathway is selected from the group consisting of glycerol-3-phosphate dehydrogenase 1 (Gpd1) (EC 1.1.1.8), glycerol-3-phosphate dehydrogenase 2 (Gpd2) (EC 1.1.1.8), a glycerol-3-phosphate phosphatase 1 (Gpp1) (EC 3.1.3.21), and glycerol-3-phosphate phosphatase 2 (Gpp2) (EC 3.1.3.21));
wherein the down-regulated enzyme in the nitrogen-assimilation pathway is glutamate dehydrogenase (Gdh) (EC 1.4.1.4); or
wherein the up-regulated enzyme in the nitrogen-assimilation pathway is at least one enzyme selected from the group consisting of glutamate dehydrogenase (Gdh) (EC 1.4.1.2), glutamate synthase (Glt) (EC 1.4.1.14), and glutamine synthase (Gln) (EC 6.3.1.2); an ammonium transporter; a urea-amido lyase (EC 6.3.4.6); and a urea transporter.

217. The recombinant yeast of claim 216, wherein the enzyme in the glycerol-production pathway is selected from the group consisting of glycerol-3-phosphate dehydrogenase 1 (Gpd1) (EC 1.1.1.8) and glycerol-3-phosphate dehydrogenase 2 (Gpd2) (EC 1.1.1.8).

218. The recombinant yeast of claim 216, wherein the enzyme in the glycerol production pathway is encoded by a polypeptide sequence at least 80% identical to the polypeptide sequence encoded by: SEQ ID NO: 5 (Gpd1); a polypeptide sequence at least 80% identical to the polypeptide sequence encoded by: SEQ ID NO: 7 (Gpd2); a polypeptide sequence at least 80% identical to the polypeptide sequence encoded by: SEQ ID NO: 159 (Gpp1); or a polypeptide sequence at least 80% identical to the polypeptide sequence encoded by: SEQ ID NO: 161 (Gpp2).

219. The recombinant yeast of claim 216, wherein the up-regulated enzyme in the nitrogen-assimilation pathway is a Gdh2 isolated from an organism from a genus selected from the group consisting of Saccharomyces and Neurospora (Gdh2); a Glt1 isolated from an organism from the genus Saccharomyces (Glt1); a Gln1 isolated from an organism from the genus Saccharomyces (Gln1); a MEP protein selected from the group consisting of, Mep1, Mep2, and Mep3 and isolated from an organism from a genus selected from the genus Saccharomyces; a urea-amido lyase isolated from an organism from the germs Saccharomyces; a Dur3 or a Dur4 isolated from an organism from the genus Saccharomyces;
or a Gln 3 isolated from the genus Saccharomyces.

220. The recombinant yeast of claim 216, wherein the down-regulated enzyme in the nitrogen-assimilation pathway is encoded by a polypeptide sequence at least 80% identical to a polypeptide sequence selected from the group consisting of: SEQ ID NOs: 25 and 31 (S. cerevisiae Gdh1 and Gdh3).

221. The recombinant yeast of claim 216, wherein the up-regulated enzyme in the nitrogen-assimilation pathway is encoded by a polypeptide sequence:
at least 80% identical to a polypeptide sequence selected from the group consisting of: SEQ ID NOs: 27 and 29 (Gdh2);
at least 80% identical to a polypeptide sequence encoded by SEQ ID NO: 33 (Glt1);
at least 80% identical to a polypeptide sequence encoded by SEQ ID NO: 35 (Gln1);
at least 80% identical to a polypeptide sequence encoded by SEQ ID NO: 19 (Mep1);
at least 80% identical to a polypeptide sequence encoded by SEQ ID NO: 21 (Mep2);
at least 80% identical to a polypeptide sequence encoded by SEQ ID NO: 23 (Mep3);
at least 80% identical to a polypeptide sequence encoded by SEQ ID NO: 37 (Dur1/2);
at least 80% identical to a polypeptide sequence encoded by: SEQ ID NO: 39 (Dur3); and/or
at least 80% identical to a polypeptide sequence encoded by SEQ ID NO: 156 (Gln3).

222. The recombinant yeast of claim 216, wherein the up-regulated enzyme in the nitrogen-assimilation pathway is an ammonium transporter and/or a urea transporter.

223. The recombinant yeast of claim 216, wherein the microorganism further comprises an up-regulation or down-regulation of a regulatory element.

224. The recombinant yeast of claim 223, wherein the regulatory element is selected from the group consisting of Ure2 and Aua1.

225. The recombinant yeast of claim 224, wherein the regulatory element is encoded by polypeptide sequence at least 80% identical to a polypeptide sequence encoded by a polynucleotide, sequence of SEQ ID NO: 55 (Ure2) or a polypeptide sequence at least 80% identical to a polypeptide sequence encoded by a polynucleotide sequence of SEQ ID NO: 57 (Aua1).

226. The recombinant yeast of claim 216, wherein the microorganism further comprises at least one additional up-regulated enzyme, wherein the at least one additional up-regulated enzyme is a permease; or a protease with. EC number: 3.4.23.41.

227. The recombinant yeast of claim 226, wherein the up-regulated enzyme is encoded by a polypeptide sequence at least 80% identical to a polypeptide sequence encoded by SEQ ID NO:
53; or a polypeptide sequence at least 80% identical to a polypeptide sequence selected from a group consisting of SEQ ID NOs: 41, 43, 45, 47, 49, and 51.

228. The recombinant yeast of claim 216, wherein the up-regulated enzymes are under the control of a heterologous promoter, wherein the heterologous promoter is selected from a group consisting of: TEF2 (SEQ ID NO: 58), HXT7 (SEQ ID NO: 59), ADH1 (SEQ ID NO: 60), and TP1 (SEQ ID NO: 61).

229. The recombinant yeast of claim 216, wherein the microorganism further comprises at least one engineered genetic modification that leads to the up-regulation of one or more native and/or heterologous saccharolytic enzyme, wherein the at least one saccharolytic enzyme is selected from the group consisting of amylases, cellulases, hemicellulases, cellulolytic and amylolytic accessory enzymes, inulinases, levanases, and pentose sugar utilizing enzymes.

230. The recombinant yeast of claim 216, wherein the yeast is from the genus Saccharomyces.

231. The recombinant yeast of claim 216, wherein the yeast is from the genus Saccharomyces cerevisiae.

232. The recombinant yeast of claim 216, wherein the yeast produces ethanol at a higher yield than an otherwise identical yeast lacking the genetic modifications.

233. The recombinant yeast of claim 216, wherein the yeast produces glycerol at a lower yield than an otherwise identical yeast lacking the genetic modifications.

234. A co-culture comprising at least two host cells wherein
a) one of the host cells comprises a recombinant yeast from claim 216; and,
b) another host cell that is genetically distinct from (a).

235. A method of producing ethanol comprising:
a) providing the recombinant microorganism of claim 216;
b) culturing the recombinant microorganism in the presence of a carbon containing feedstock for sufficient time to produce ethanol; and, optionally,
c) extracting the ethanol.
